SEQUENCE LISTING (71 sequences)

SEQ ID NO: 1 - DNA, 253 bp, Chlamydomonas reinhardtii
  misc_feature: RBCS2
cgcttagaagatttcgataaggcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaacggtcttaagaagtctatccgg

SEQ ID NO: 2 - DNA, 312 bp, Chlamydomonas reinhardtii
  misc_feature: Beta-2 tubulin
ctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaac

SEQ ID NO: 3 - DNA, 356 bp, Chlorella virus
  misc_feature: Promoter #1
cggggatcgcagggcatgggcattaaaagaactttatggaatcaaaaatcttagtgaatttccaccacaggtatatagtcttcaggacgctaacgatgatatcaacgattgtatcaaaggttatcgtttgaggcactcatatcaggtagtttctacacagaaacttgaacaacgcctgggaaaagatcctgagcatagtaacttatatactagcagatgttgtaacgatgctttatatgaatatgaattagcacaacgacaactacaaaaacaacttgatgaatttgacgaagatgggtatgatttttttcaggcacgtataaatacattagatccgtcgacctgcagccaagctt

SEQ ID NO: 4 - DNA, 207 bp, Chlorella virus
  misc_feature: Promoter #2
cccggggatcatcgaaagcaactgccgcattcgaaacttcgactgcctcgttataaaggttagtgaaagccattgtatgttattactgagttatttaatttagcttgcttaaatgcttatcgtgttgatatgataaatgacaaatgatacgctgtatcaacatctcaaaagattaatacgaagatccgtcgacctgcagccaagctt

SEQ ID NO: 5 - DNA, 277 bp, Chlorella virus
  misc_feature: Promoter #3
cccggggatctgcgtattgcgggacttttgagcattttccagaacggattgccgggacgtatactgaacctccagtccctttgctcgtcgtatttcccataatatacatatacactattttaattatttacaccggttgttgctgagtgatacaatgcaaattccctccaccgaggaggatcgcgaactgtccaaatgtcttctttctgcagctccatacggagtcgttaggaaacattcacttaattataggatccgtcgacctgcagccaagctt

SEQ ID NO: 6 - DNA, 489 bp, Rhodella reticulata
  misc_feature: Phycoerythrin beta subunit promoter
tttttatagatcatccaattattttttcattagatattgtatatcaataatttggcatatgttttgtagtatacgggttatgatattgcaatatatgtacaacattggtaatttttggacttacatatatatcaattatatcaatgacaatgtaatatattggttgatagatcaataaacatctttaataagatctgttaaaattcaaatatagactttctgtattataagtagttttcttatattactatagacgtagaacgatcaaaaaaaaataaatatggacatgacttgattcaatatggaagacggggtatgagaaatatcgtgttgcactcaatatagaattgacgtatttttaatgcagtgcccgttatatattgcgtaacaaagattaaaagtatattatatattataatactagtagaccagcaaatataaaattatgctgaaacaataataccctttaaagttttaaggagccttttc

SEQ ID NO: 7 - DNA, 543 bp, Porphyridium sp.
  misc_feature: AHAS promoter
attatttaacaattggaaacttagttaattagggtaaattatattaacccttatgaaccaaaataatttggtttcaaaaaaaactaacttatgaattaaaattgaaatattttctacatcataataattttaattctaaatagaattttagataagggatctaagataacaaaaaaatcaatttaagtaataaagaaaatgtgattacaaaatttttgatattaaactatagtatttacaaattattatcaaaaattacttatccatttgaggaaaagactgaacctttaaacatatttgtttatgcgattttagatcattcaagttagcgagctgtatgaaatgaaagtttcatgtacagttcttaagtagagatgtatatatgttaatagaaatattatttgcatcgactataatcaattctgaagacttcaaaataaaacctgttatacgtgctatactagagatggttgatgaaataaatcaaccaggtattattacagactgaactgaactaaaaaaattcatataatttagcgtact

SEQ ID NO: 8 - DNA, 799 bp, Porphyridium purpureum
  misc_feature: Carbonic anhydrase pca2 promoter
gcacacgagtgttgtggcgttgtcgcagcaggtttgggggcgcgagagcgcacgacgcttgtgtgtgtgtgtgtgtgtggaccgcaaccaccctcgcgacgcggcattgccgtgcgtgccgtcgcggctgcgtggttcgtggtgtgatattctaaacgcatgtgggttgggtgtgggtgttgttctgtgtccatcaggcgatggacacagccgccactgaagtgtcactgaattaagcgcggtgcattttgcacgtggcttttgtgtgggtgtgtgtgtatgtgtcctgctcggcttgtatcgacatcctccttcgtttttctcgtacggggcttttgtgtttcctttggtacgtggtgagcgttttttggggtgttgccggacatgatggtgttgtgtttgtgagtttgggagtgtgagactgggagcgacggtgaagccgcatgaatcgtggagcgcaaaatgcaagttgactggagccatcgcgatgcttttggcgttttgcgcatgtgatcacaatctcctcggaatggtccaaaatggatcgaactggctcgccccccaatctgtgcgctttcggcctgttcggacatgccggtttcgcggtgcgcagcatgtggctcgcgcatggtaggggatgttggcgcggggcataaataggctgcgacaacttgccgcttccccttcatcgcacacctcaggcaggaggaagtggtggaaaagactggtgcaggagaggattttgcaggagaggaaggagagggagaggcgtgtcgtgcttgccactgcgatagtcacc

SEQ ID NO: 9 - DNA, 848 bp, Porphyridium purpureum
  misc_feature: Carbonic anhydrase pca1 promoter
gcgtgcgtcaagcacattggggcaactcgggcaaccgacgcagccacgcacacgagtgttgtggcgttgtcgtagcaggtttgggggcgcgagagcgcacgacgcgtgtgtgtgtgtgtgtgtgtggaccgcaaccaccctcgcgacgcggcattgccgtgcctgccgttgcggctgcgtggttcgtggtgtgatattctaaacgcatgtgggttgggtgttggtgttgttctgtgtccatcaggcgatggacacagccgccactgaagtgtcactgaattaagcgcggtgcattttgcacgtggcttttgtgtgtgtgtgtttgtgtctatgtgtcctgctcggtttgtatcgacgtcctccttcgtttttttcgcacggggcttttgtctttcctttggtacgtggtgagcgttttttggggtgttgccggacatgatggtgttgtgtttgtgagtttgagagtgagactgggagcgacggtgaagccgcatgaatcgtggagcgcaaaatgcaagttgactggagccatcgcgatgcttttggcgttttgcgcatgtgatcacaatctcctcggaatggtccaaaatggatcgaactggctcgccccccaatctgtgcgctttcggcctgttcggacatgccggtttcgtggtgcgcagcatgtggctcgcgcatggtaggggatgttggcgcggggcataaataggctgcgacaacttgccgcttccccttccctgcacgcctcaggcaggaagaagtggtggaaaagactggtgcaggagaggatcttgcaggagaggaaggagagggagaggcgtgtcgtgcttgccactgcaatcgtcacc

SEQ ID NO: 10 - protein, 587 aa, Porphyridium sp.
  MISC_FEATURE: Sulfometuron resistance - acetohydroxy acid synthase
MTHIEKSNYQEQTGAFALLDSLVRHKVKHIFGYPGGAILPIYDELYKWEEQGYIKHILVRHEQGAAHAADGYARATGEVGVCFATSGPGATNLVTGIATAHMDSIPIVIITGQVGRSFIGTDAFQEVDIFGITLPIVKHSYVIRDPRDIPRIVAEAFSIAKQGRPGPVLIDVPKDVGLETFEYQYVNPGEARIPGFRDLVAPSSRQIIHSIQLIQEANQPLLYVGGGAITSGAHDLIYKLVNQYKIPITTTLMGKGIIDEQNPLALGMLGMHGTAYANFAVSECDLLITLGARFDDRVTGKLDEFACNAKVIHVDIDPAEVGKNRIPQVAIVGDISLVLEQWLLYLDRNLQLDDSHLRSWHERIFRWRQEYPLIVPKLVQTLSPQEIIANISQIMPDAYFSTDVGQHQMWAAQFVKTLPRRWLSSSGLGTMGYGLPAAIGAKIAYPESPVVCITGDSSFQMNIQELGTIAQYKLDIKIIIINNKWQGMVRQSQQAFYGARYSHSRMEDGAPNFVALAKSFGIDGQSISTRQEMDSLFNTIIKYKGPMVIDCKVIEDENCYPMVAPGKSNAQMIGLDKSNNEIIKIKE

SEQ ID NO: 11 - protein, 129 aa, Streptoalloteichus hindustanus
  MISC_FEATURE: Zeocin resistance- ble protein
MARMAKLTSAVPVLTARDVAGAVEFWTDRLGFSRDFVEDDFAGVVRDDVTLFISAVQDQDQVVPDNTLAWVWVRGLDELYAEWSEVVSTNFRDASGPAMTEIGEQPWGREFALRDPAGNCVHFVAEEQD

SEQ ID NO: 12 - protein, 763 aa, Chlamydomonas reinhardtii
  MISC_FEATURE: Zeaxanthin epoxidase
MLASTYTPCGVRQVAGRTVAVPSSLVAPVAVARSLGLAPYVPVCEPSAALPACQQPSGRRHVQTAATLRADNPSSVAQLVHQNGKGMKVIIAGAGIGGLVLAVALLKQGFQVQVFERDLTAIRGEGKYRGPIQVQSNALAALEAIDPEVAAEVLREGCITGDRINGLCDGLTGEWYVKFDTFHPAVSKGLPVTRVISRLTLQQILAKAVERYGGPGTIQNGCNVTEFTERRNDTTGNNEVTVQLEDGRTFAADVLVGADGIWSKIRKQLIGETKANYSGYTCYTGISDFTPADIDIVGYRVFLGNGQYFVSSDVGNGKMQWYGFHKEPSGGTDPEGSRKARLLQIFGHWNDNVVDLIKATPEEDVLRRDIFDRPPIFTWSKGRVALLGDSAHAMQPNLGQGGCMAIEDAYELAIDLSRAVSDKAGNAAAVDVEGVLRSYQDSRILRVSAIHGMAGMAAFMASTYKCYLGEGWSKWVEGLRIPHPGRVVGRLVMLLTMPSVLEWVLGGNTDHVAPHRTSYCSLGDKPKAFPESRFPEFMNNDASIIRSSHADWLLVAERDAATAAAANVNAATGSSAAAAAAADVNSSCQCKGIYMADSAALVGRCGATSRPALAVDDVHVAESHAQVWRGLAGLPPSSSSASTAAASASAASSAASGTASTLGSSEGYWLRDLGSGRGTWVNGKRLPDGATVQLWPGDAVEFGRHPSHEVFKVKMQHVTLRSDELSGQAYTTLMVGKIRNNDYVMPESRPDGGSQQPGRLVTA

SEQ ID NO: 13 - protein, 524 aa, Arabidopsis thaliana
  MISC_FEATURE: Lycopene epsilon cyclase
MECVGARNFAAMAVSTFPSWSCRRKFPVVKRYSYRNIRFGLCSVRASGGGSSGSESCVAVREDFADEEDFVKAGGSEILFVQMQQNKDMDEQSKLVDKLPPISIGDGALDLVVIGCGPAGLALAAESAKLGLKVGLIGPDLPFTNNYGVWEDEFNDLGLQKCIEHVWRETIVYLDDDKPITIGRAYGRVSRRLLHEELLRRCVESGVSYLSSKVDSITEASDGLRLVACDDNNVIPCRLATVASGAASGKLLQYEVGGPRVCVQTAYGVEVEVENSPYDPDQMVFMDYRDYTNEKVRSLEAEYPTFLYAMPMTKSRLFFEETCLASKDVMPFDLLKTKLMLRLDTLGIRILKTYEEEWSYIPVGGSLPNTEQKNLAFGAAASMVHPATGYSVVRSLSEAPKYASVIAEILREETTKQINSNISRQAWDTLWPPERKRQRAFFLFGLALIVQFDTEGIRSFFRTFFRLPKWMWQGFLGSTLTSGDLVLFALYMFVISPNNLRKGLINHLISDPTGATMIKTYLKV

SEQ ID NO: 14 - protein, 144 aa, Haloarcula hispanica
  MISC_FEATURE: Superoxide Dismutase
GYVNGLESAEETLAENRESGDFGSSAAAMGNVTHNGCGHYLHTLFWENMDPNGGGEPEGELLDRIEEDFGSYEGWKGEFEAAASAAGGWALLVYDPVAKQLRNVPVDKHDQGALWGSHPILALDVWEHSYYYDYGPARGDFIDA

SEQ ID NO: 15 - protein, 154 aa, Homo sapiens [sequence omitted]

SEQ ID NO: 16 - protein, 711 aa, Artificial
  Synthetic Superoxide Dismutase-Polysaccharide binding protein fusion AAV48590-NP_000445
MARMVVAAVAVMAVLSVALAQFIPDVDITWKVPMTLTVQNLSIFTGPNQFGRGIPSPSAIGGGNGLDIVGGGGSLYISPTGGQVQYSRGSNNFGNQVAFTRVRKNGNNESDFATVFVGGTTPSFVIVGDSTENEVSFWTNNKVVVNSQGFIPPNGNSAGGNSQYTFVNGITGTAGAPVGGTVIRQVSAWREIFNTAGNCVKSFGLVVRGTGNQGLVQGVEYDGYVAIDSNGSFAISGYSPAVNNAPGFGKNFAAARTGNFFAVSSESGVIVMSIPVDNAGCTLSFSVAYTITPGAGRVSGVSLAQDNEFYAAVGIPGAGPGEVRIYRLDGGGATTLVQTLSPPDDIPELPIVANQRFGEMVRFGANSETNYVAVGSPGYAAEGLALFYTAEPGLTPNDPDEGLLTLLAYSNSSEIPANGGLGEFMTASNCRQFVFGEPSVDSVVTFLASIGAYYEDYCTCERENIFDQGIMFPVPNFPGESPTTCRSSIYEFRFNCLMEGAPSICTYSERPTYEWTEEVVDPDNTPCELVSRIQRRLSQSNCFQDYVTLQVVGAGAGMATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQ

SEQ ID NO: 17 - protein, 191 aa, Homo sapiens
  MISC_FEATURE: Human growth hormone
FPTIPLSRLFQNAMLRAHRLHQLAFDTYEEFEEAYIPKEQKYSFLQAPQASLCFSESIPTPSNREQAQQKSNLQLLRISLLLIQSWLEPVGFLRSVFANSLVYGASDSDVYDLLKDLEEGIQTLMGRLEDGSPRTGQAFKQTYAKFDANSHNDDALLKNYGLLYCFRKDMDKVETFLRIVQCRSVEGSCGF

SEQ ID NO: 18 - protein, 217 aa, Bos taurus
  MISC_FEATURE: Bovine growth hormone
MMAAGPRTSLLLAFALLCLPWTQVVGAFPAMSLSGLFANAVLRAQHLHQLAADTFKEFERTYIPEGQRYSIQNTQVAFCFSETIPAPTGKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFRKDLHKTETYLRVMKCRRFGEASCAF

SEQ ID NO: 19 - protein, 216 aa, Sus scrofa
  MISC_FEATURE: Porcine growth hormone
MAAGPRTSALLAFALLCLPWTREVGAFPAMPLSSLFANAVLRAQHLHQLAADTYKEFERAYIPEGQRYSIQNAQAAFCFSETIPAPTGKDEAQQRSDVELLRFSLLLIQSWLGPVQFLSRVFTNSLVFGTSDRVYEKLKDLEEGIQALMRELEDGSPRAGQILKQTYDKFDTNLRSDDALLKNYGLLSCFKKDLHKAETYLRVMKCRRFVESSCAF

SEQ ID NO: 20 - protein, 534 aa, Parachlorella kessleri
  MISC_FEATURE: Chlorella hexose transporter
MAGGAIVASGGASRSSEYQGGLTAYVLLVALVAACGGMLLGYDNGVTGGVASMEQFERKFFPDVYEKKQQIVETSPYCTYDNPKLQLFVSSLFLAGLISCIFSAWITRNWGRKASMGIGGIFFIAAGGLVNAFAQDIAMLIVGRVLLGFGVGLGSQVVPQYLSEVAPFSHRGMLNIGYQLFVTIGILIAGLVNYGVRNWDNGWRLSLGLAAVPGLILLLGAIVLPESPNFLVEKGRTDQGRRILEKLRGTSHVEAEFADIVAAVEIARPITMRQSWRSLFTRRYMPQLLTSFVIQFFQQFTGINAIIFYVPVLFSSLGSASSAALLNTVVVGAVNVGSTMIAVLLSDKFGRRFLLIEGGITCCLAMLAAGITLGVEFGQYGTEDLPHPVSAGVLAVICIFIAGFAWSWGPMGWLIPSEIFTLETRPAGTAVAVMGNFLFSFVIGQAFVSMLCAMKFGVFLFFAGWLVIMVLCAIFLLPETKGVPIERVQALYARHWFWKKVMGPAAQEIIAEDEKRVAASQAIMKEERISQTMK

SEQ ID NO: 21 - DNA, 1605 bp, Artificial
  Synthetic Chlorella hexose transporter from Q39525 in Porphyridium sp. preferred codons
atggcgggcggcgccattgttgccagcggcggcgccagccgttcgagcgagtaccagggcggcctgaccgcctacgttctgctcgtggcgctggttgccgcctgcggcggcatgctgctgggctacgacaacggcgttaccggcggcgttgccagcatggagcagttcgagcgcaagttcttcccggacgtgtacgagaagaagcagcagattgtcgagaccagcccgtactgcacctacgacaacccgaagctccagctgttcgtgtcgagcctgttcctggcgggcctgattagctgcattttctcggcgtggattacccgcaactggggccgcaaggcgagcatgggcattggcggcattttcttcattgccgccggtggcctggttaacgccttcgcccaggacattgccatgctgattgtgggccgcgtcctgctgggcttcggcgttggcctgggcagccaggtggtgccacagtacctgagcgaggtggcgccattcagccatcgcggcatgctcaacattggctaccagctcttcgtgaccattggcattctgattgccggcctggtgaactacggcgtgcgcaactgggacaacggttggcgcctgagcctgggcctggcggcggttccaggcctgattctgctgctcggcgccatcgttctgccggagagcccgaacttcctggtggagaagggccgcaccgaccagggccgccgcattctggagaagctgcgcggcaccagccatgttgaggcggagttcgccgacattgtggcggcggtggagattgcccgcccaattaccatgcgccagagctggcgctcgctgttcacccgccgctacatgccacagctgctgaccagcttcgtgattcagttcttccagcagttcaccggcattaacgccatcattttctacgtgccggtgctgttcagcagcctgggctcggcgtcctcggcggcgctgctgaacaccgtggttgtgggcgccgtgaacgtgggcagcaccatgattgccgtgctgctgtcggacaagttcggccgccgcttcctgctgattgagggcggcattacctgctgcctggcgatgctggcggcgggcattacgctgggcgtggagttcggccagtacggcaccgaggacctgccacatccagtgtcggcgggcgtgctggcggtgatttgcattttcattgccggcttcgcctggagctggggcccaatgggctggctgattccgagcgagattttcaccctggagacccgcccagcgggcacggcggttgccgtgatgggcaacttcctgttctcgttcgtgattggccaggccttcgtgtcgatgctgtgcgcgatgaagttcggcgtgttcctgttcttcgccggctggctggtgattatggtgctgtgcgccattttcctgctgccggagaccaagggcgtgccgattgagcgcgtgcaggcgctgtacgcccgccactggttctggaagaaggtgatgggcccagcggcccaggagattattgccgaggacgagaagcgcgttgcggcgagccaggcgattatgaaggaggagcgcattagccagaccatgaagtaa

SEQ ID NO: 22 - protein, 541 aa, Saccharomyces cerevisiae
  MISC_FEATURE: Yeast hxt2
MSEFATSRVESGSQQTSIHSTPIVQKLETDESPIQTKSEYTNAELPAKPIAAYWTVICLCLMIAFGGFVFGWDTGTISGFVNQTDFKRRFGQMKSDGTYYLSDVRTGLIVGIFNIGCAFGGLTLGRLGDMYGRRIGLMCVVLVYIVGIVIQIASSDKWYQYFIGRIISGMGVGGIAVLSPTLISETAPKHIRGTCVSFYQLMITLGIFLGYCTNYGTKDYSNSVQWRVPLGLNFAFAIFMIAGMLMVPESPRFLVEKGRYEDAKRSLAKSNKVTIEDPSIVAEMDTIMANVETERLAGNASWGELFSNKGAILPRVIMGIMIQSLQQLTGNNYFFYYGTTIFNAVGMKDSFQTSIVLGIVNFASTFVALYTVDKFGRRKCLLGGSASMAICFVIFSTVGVTSLYPNGKDQPSSKAAGNVMIVFTCLFIFFFAISWAPIAYVIVAESYPLRVKNRAMAIAVGANWIWGFLIGFFTPFITSAIGFSYGYVFMGCLVFSFFYVFFFVCETKGLTLEEVNEMYVEGVKPWKSGSWISKEKRVSEE

SEQ ID NO: 23 - DNA, 1626 bp, Artificial
  Synthetic yeast hxt2 from P23585 in Porphyridium sp. preferred codons
atgagcgagttcgccacctcgcgcgttgagagcggcagccagcagaccagcattcacagcaccccgattgtccagaagctggagaccgacgagagcccgattcagaccaagagcgagtacaccaacgccgagctgccggcgaagccaattgccgcctactggaccgtgatttgcctgtgcctgatgattgccttcggcggcttcgtgttcggctgggacaccggcaccatttcgggcttcgtgaaccagaccgacttcaagcgccgcttcggccagatgaagagcgacggcacctactacctgagcgacgtgcgcaccggcctgattgtgggcattttcaacattggctgcgccttcggtggcctgaccctgggccgcctgggcgacatgtacggccgccgcattggcctgatgtgcgtggtgctggtgtacattgtcggcatcgtgattcagattgccagcagcgacaagtggtatcagtacttcattggccgcattattagcggcatgggcgtgggcggcattgccgttctgagcccgaccctgattagcgagaccgccccgaagcatattcgcggcacctgcgtgtcgttctaccagctgatgattaccctgggcatcttcctgggctactgcaccaactacggcaccaaggactacagcaacagcgtccagtggcgcgttccactgggcctgaacttcgccttcgccattttcatgattgccggcatgctgatggtgccagagagcccacgcttcctggttgagaagggccgctacgaggacgccaagcgctcgctggcgaagagcaacaaggtgaccattgaggacccgagcattgtggcggagatggacaccattatggcgaacgtggagaccgagcgcctggcgggcaacgccagctggggcgagctgttcagcaacaagggcgccattctgccgcgcgtgattatgggcattatgatccagagcctccagcagctgaccggcaacaactacttcttctactacggcacgaccattttcaacgccgtgggcatgaaggacagcttccagacctcgattgtgctgggcattgtcaacttcgccagcaccttcgtggcgctgtacaccgtggacaagttcggccgccgcaagtgcctgctgggcggctcggcgagcatggcgatttgcttcgtgattttcagcaccgtgggcgtgaccagcctgtacccgaacggcaaggaccagccgagcagcaaggcggccggcaacgtgatgattgtgttcacctgcctgttcatcttcttcttcgccattagctgggcgccgattgcctacgtgatcgtggcggagagctacccactgcgcgtgaagaaccgcgcgatggcgattgccgttggcgccaactggatttggggcttcctgattggcttcttcaccccgttcattacctcggcgattggcttcagctacggctacgtgttcatgggctgcctggtgttctcgttcttctacgtgttcttcttcgtgtgcgagaccaagggcctgacgctggaggaggtgaacgagatgtacgtggagggcgtgaagccgtggaagagcggctcgtggattagcaaggagaagcgcgtttcggaggagtaa

SEQ ID NO: 24 - protein, 492 aa, Homo sapiens
  MISC_FEATURE: Human GLUT1
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTAFRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 25 - DNA, 1479 bp, Artificial
  Synthetic human GLUT1 from AAA52571 in Porphyridium sp. preferred codons
atggagccgagcagcaagaagctgaccggccgcctgatgctggcggttggcggcgccgttctgggcagcctccagttcggctacaacaccggcgtgattaacgccccacagaaggtgatcgaggagttctacaaccagacctgggtccaccgctacggcgagagcattctgccgaccaccctgaccacgctgtggagcctgagcgtggcgattttcagcgtgggcggcatgattggcagcttctcggtgggcctgttcgtgaaccgcttcggccgccgcaacagcatgctgatgatgaacctgctggccttcgtgtcggcggtgctgatgggcttcagcaagctgggcaagagcttcgagatgctgattctgggccgcttcattattggcgtgtactgcggcctgaccaccggcttcgtgccgatgtacgtgggcgaggtgtcgccaacggcgttccgcggcgcgctgggcaccctccatcagctgggcattgttgtgggcattctgattgcccaggtgttcggcctggacagcattatgggcaacaaggacctgtggccgctgctgctgtcgattattttcattccggcgctgctccagtgcattgtgctgccgttctgcccagagagcccacgcttcctgctgattaaccgcaacgaggagaaccgcgcgaagagcgtgctgaagaagctgcgcggcacggcggacgttacccacgacctccaggagatgaaggaggagagccgccagatgatgcgcgagaagaaggtgaccattctggagctgttccgctcgccagcgtaccgccagccgattctgatcgccgtggtgctccagctgtcccagcagctgtcgggcattaacgccgtgttctactacagcaccagcattttcgagaaggcgggcgtccagcagccagtgtacgccaccattggcagcggcattgtgaacaccgccttcaccgtggtgtcgctgttcgtggttgagcgcgcgggccgccgcacgctccatctgattggcctggcgggcatggcgggctgcgcgattctgatgaccattgccctggcgctgctggagcagctgccgtggatgagctacctgagcattgtggcgatcttcggcttcgtggcgttcttcgaggttggcccaggcccgattccgtggttcattgtggcggagctgttcagccagggcccacgcccagcggcgattgccgttgccggcttctcgaactggaccagcaacttcattgtgggcatgtgcttccagtacgtcgagcagctgtgcggcccgtacgtgttcattatcttcaccgtgctgctggtcctcttcttcatcttcacctacttcaaggtgccggagaccaagggccgcaccttcgacgagattgccagcggcttccgccagggcggcgccagccagagcgacaagaccccggaggagctgttccatccactgggcgccgacagccaggtgtaa

SEQ ID NO: 26 - protein, 1039 aa, Artificial
  Synthetic transporter sequence
MQAKASTSPLGDSIEPRTENLEYATEQKESFVPRRAFGTAAERARRNLNAKLANPLSGYSHEELRRQGINFAITHQIGDEGDIRAFGLGAMLAQAPEKFENVPGLTVQELEVLRHEFEHRWSQPWTMYLVIILCSLSAAVQGMDETVVNGAQIFYKHQFGIADENISRHNWISGLVNAAPYLCCAIVGCWLTVPFNSWFGRRGTIFITCIFSATTCLWQGCCSTWWSLFIARFALGFGIGPKSATVPVYAAETGGLLLELCLVPDSSGIVGLNWRLMLASALVPAVIVCCFVFMCPESPRWYMSRNLYDRAYQSMCSLRFNKVQAARDMYYMYTLLEAEKSMKLGQNKLLELINVPRNRRAMFASEIVMFMQQFCGVNVLAYYSSEIFLQTASEHSKLTVSNQRKALTASLGWGLINWLFAIPAVYTIDTFGRRNLLLSTFPLMALSMFGPPSSFFFFFFFTKWVNFGLFLVAVFIFIAAYSPANGPVPWVYCPEIFPLYVRAQGMAITTFFNYLFNFVVSYSWPDMLQKLKAQGGYGFYAGAIAVGWVLLFFFMPETKGYTLEQMGMVFEHSLGEIARYHWKCGIRNIRKLFGLPTSSEPLASPYNKKLNLKMHGVEERVIQRQRLLPQQQRRNQSKSELPDGKPSVVSVILGLNAIESREIAQIIFYNAKMDASENQAQAQQQTPQKPTYQNGVRTNGRAFNSPNWRVKREESPSGSRSPSQDTQNGSPRRTPGFGRQNREVPQAISEGRRLYVGNMPYTAKMEDVQELFTRGGFEVVRIDISIDPFSGRNPSYCFVDLSTKELAERAMAELDGGDLLGRPVRIKPGVVKSASERQPQQRTGMGAGTGSIGDGMSSGSPRANRAGSSPLNADRWRRDDNLTSASTTPTKLGNMSTYNPKADPSKRLYVGGLPRLTDPDAISSNITQFFKGYNLTNISKLFTPHPAKRFEPGDHYYLFVDFETVEETQNAMAALNGAEGPWGAAIRVQRARGETWKNTDSNNTSEERRPAAGRWGPTTRRQDVASTPAPASGEAAVQA

SEQ ID NO: 27 - protein, 661 aa, Artificial
  Synthetic transporter sequence
MVEKSSDPEVPSLSHHESSISIEKQGDAATAREWAQDVNSTTTNTKLKNPLAGLTREQLLNDVEAFAKEKDLEHILDDLRKGALVAQDPREFEQMDALTESEKELLRREKTHRWSQPFMMYFMTSESSRYPPTEFGFNPACQSSVLDLLSCREWIRLLSTVRRSMYSSITHLSYAKQSRFYFAEFNVTDTWMQGLLNGAPYLCSAVIGCWTTAPLNRWFGRRGCIFISCFISFASSFWMAAAHTWWNLLLGRFLLGFAVGAKSTTTPVYGAECSPANIRGALVMMWQMWTAFGIMLGYIASVAFMDVTHPTIPGFNWRLMLGSTAIPPFFVCIQVYTVPESPRWLIKRRRYEDAKRNLFKLRRTAETAERDFVRIKKGVEEDEILQKGKNLLVEVIPVPYIRRALLIGIMEMLFQQMSGMNVFMNYIDEVFEENINMGARTSVAVSLFPGFVNMVATVIVYFTIDRYGRRTLQLVTFPVMFLMLLMVLFSFYGDKKVNLAFFIIGVVFFIVAYSPGAGPVPWTFCAEVFPTYVRAAGTTITTFFVNAFNFALSFSWPSMKAAWGPQGGFGFYAGFNFLGIVMQFLFLPETKGFTLEQMRVVFEEGLFTIAAYHCRAGWRSLRKLLGLSVPDTPLVSPYDKAFAIDRAKREEEMMHAGEVSK

SEQ ID NO: 28 - protein, 552 aa, Porphyridium sp.
  MISC_FEATURE: Protein associated with polysaccharide
MARMVVAAVAVMAVLSVALAQFIPDVDITWKVPMTLTVQNLSIFTGPNQFGRGIPSPSAIGGGNGLDIVGGGGSLYISPTGGQVQYSRGSNNFGNQVAFTRVRKNGNNESDFATVFVGGTTPSFVIVGDSTENEVSFWTNNKVVVNSQGFIPPNGNSAGGNSQYTFVNGITGTAGAPVGGTVIRQVSAWREIFNTAGNCVKSFGLVVRGTGNQGLVQGVEYDGYVAIDSNGSFAISGYSPAVNNAPGFGKNFAAARTGNFFAVSSESGVIVMSIPVDNAGCTLSFSVAYTITPGAGRVSGVSLAQDNEFYAAVGIPGAGPGEVRIYRLDGGGATTLVQTLSPPDDIPELPIVANQRFGEMVRFGANSETNYVAVGSPGYAAEGLALFYTAEPGLTPNDPDEGLLTLLAYSNSSEIPANGGLGEFMTASNCRQFVFGEPSVDSVVTFLASIGAYYEDYCTCERENIFDQGIMFPVPNFPGESPTTCRSSIYEFRFNCLMEGAPSICTYSERPTYEWTEEVVDPDNTPCELVSRIQRRLSQSNCFQDYVTLQVV

SEQ ID NO: 29 - protein, 523 aa, Nicotiana tabacum
  MISC_FEATURE: Glucose transporter
MAGGGGIGPGNGKEYPGNLTLYVTVTCIVAAMGGLIFGYDIGISGGVTSMDSFLSRFFPSVFRKQKADDSTNQYCKFDSQTLTMFTSSLYLAALLSSLVASTVTRKLGRRLSMLCGGVLFCAGALINGFAQNVAMLIVGRILLGFGIGFANQSVPLYLSEMAPYKYRGALNLGFQLSITIGILVANVLNYFFAKIHWGWRLSLGGAMVPALIITIGSLFLPETPNSMIERGNHDEAKARLKRIRGIDDVDEEFNDLVVASEASRKIENPWRNLLQRKYRPHLTMAIMIPFFQQLTGINVIMFYAPVLFKTIGFGADASLMSAVITGGVNVLATVVSIYYVDKLGRRFLFLEGGIQMLICQIAVSICIAIKFGVNGTPGDLPKWYAIVVVIFICVYVAGFAWSWGPLGWLVPSEIFPLEIRSAAQSINVSVNMIFTFIVAQVFLTMLCHLKFGLFLFFAFFVVIMTVFIYFFLPETKNIPIEEMVIVWKEHWFWSKFMTEVDYPGTRNGTSVEMSKGSAGYKIV

SEQ ID NO: 30 - protein, 522 aa, Arabidopsis thaliana
  MISC_FEATURE: Glucose transporter GlcGalFrc
MPAGGFVVGDGQKAYPGKLTPFVLFTCVVAAMGGLIFGYDIGISGGVTSMPSFLKRFFPSVYRKQQEDASTNQYCQYDSPTLTMFTSSLYLAALISSLVASTVTRKFGRRLSMLFGGILFCAGALINGFAKHVWMLIVGRILLGFGIGFANQAVPLYLSEMAPYKYRGALNIGFQLSITIGILVAEVLNYFFAKIKGGWGWRLSLGGAVVPALIITIGSLVLPDTPNSMIERGQHEEAKTKLRRIRGVDDVSQEFDDLVAASKESQSIEHPWRNLLRRKYRPHLTMAVMIPFFQQLTGINVIMFYAPVLFNTIGFTTDASLMSAVVTGSVNVGATLVSIYGVDRWGRRFLFLEGGTQMLICQAVVAACIGAKFGVDGTPGELPKWYAIVVVTFICIYVAGFAWSWGPLGWLVPSEIFPLEIRSAAQSITVSVNMIFTFIIAQIFLTMLCHLKFGLFLVFAFFVVVMSIFVYIFLPETKGIPIEEMGQVWRSHWYWSRFVEDGEYGNALEMGKNSNQAGTKHV

SEQ ID NO: 31 - protein, 516 aa, Vivica faba
  MISC_FEATURE: Glucose transport protein
MPAAGIPIGAGNKEYPGNLTPFVTITCVVAAMGGLIFGYDIGISGGVTSMNPFLEKFFPAVYRKKNAQHSKNQYCQYDSETLTLFTSSLYLAALLSSVVASTITRRFGRKLSMLFGGLLFLVGALINGLAQNVAMLIVGRILLGFGIGFANQSVPLYLSEMAPYKYRGALNIGFQLSITIGILVANILNYFFAKIKGGWGWRLSLGGAMVPALIITIGSLILPDTPNSMIERGDRDGAKAQLKRIRGVEDVDEEFNDLVAASETSMQVENPWRNLLQRKYRPQLTMAVLIPFFQQFTGINVIMFYAPVLFNSIGFKDDASLMSAVITGVVNVVATCVSIYGVDKWGRRALFLEGGVQMLICQVAVAVSIAAKFGTSGEPGDLPKWYAIVVVLFICIYVAGFAWSWGPLGWLVPSEIFPLEIRSAAQSVNVSVNMLFTFLVAQIFLTMLCHMKFGLFLFFAFFVVVMTIYIYTMLPETKGIPIEEMDRVWKSHPYWSRFVEHDDNGVEMAKGGVKNV

SEQ ID NO: 32 - protein, 540 aa, Parachlorella kessleri
  MISC_FEATURE: Galactose-H+ symporter
MAGGGPVASTTTNRASQYGYARGGLNWYIFIVALTAGSGGLLFGYDIGVTGGVTSMPEFLQKFFPSIYDRTQQPSDSKDPYCTYDDQKLQLFTSSFFLAGMFVSFFAGSVVRRWGRKPTMLIASVLFLAGAGLNAGAQDLAMLVIGRVLLGFGVGGGNNAVPLYLSECAPPKYRGGLNMMFQLAVTIGIIVAQLVNYGTQTMNNGWRLSLGLAGVPAIILLIGSLLLPETPNSLIERGHRRRGRAVLARLRRTEAVDTEFEDICAAAEESTRYTLRQSWAALFSRQYSPMLIVTSLIAMLQQLTGINAIMFYVPVLFSSFGTARHAALLNTVIIGAVNVAATFVSIFSVDKFGRRGLFLEGGIQMFIGQVVTAAVLGVELNKYGTNLPSSTAAGVLVVICVYVAAFAWSWGPLGWLVPSEIQTLETRGAGMSMAVIVNFLFSFVIGQAFLSMMCAMRWGVFLFFAGWVVIMTFFVYFCLPETKGVPVETVPTMFARHWLWGRVMGEKGRALVAADEARKAGTVAFKVESGSEDGKPASDQ

SEQ ID NO: 33 - protein, 383 aa, Arabidopsis thaliana
  MISC_FEATURE: ATSTP2 carbohydrate transporter
MAVGSMNVEEGTKAFPAKLTGQVFLCCVIAAVGGLMFGYDIGISGGVTSMDTFLLDFFPHVYEKKHRVHENNYCKFDDQLLQLFTSSLYLAGIFASFISSYVSRAFGRKPTIMLASIFFLVGAILNLSAQELGMLIGGRILLGFGIGFGNQTVPLFISEIAPARYRGGLNVMFQFLITIGILAASYVNYLTSTLKNGWRYSLGGAAVPALILLIGSFFIHETPASLIERGKDEKGKQVLRKIRGIEDIELEFNEIKYATEVATKVKSPFKELFTKSENRPPLVCGTLLQFFQQFTGINVVMFYAPVLFQTMGSGDNASLISTVVTNGVNAIATVISLLVVDFAGRRCLLMEGALQMTATQMTIGGILLAHLKLVGPITGHAVR

SEQ ID NO: 34 - protein, 514 aa, Arabidopsis thaliana
  MISC_FEATURE: STP4
MAGGFVSQTPGVRNYNYKLTPKVFVTCFIGAFGGLIFGYDLGISGGVTSMEPFLEEFFPYVYKKMKSAHENEYCRFDSQLLTLFTSSLYVAALVSSLFASTITRVFGRKWSMFLGGFTFFIGSAFNGFAQNIAMLLIGRILLGFGVGFANQSVPVYLSEMAPPNLRGAFNNGFQVAIIFGIVVATIINYFTAQMKGNIGWRISLGLACVPAVMIMIGALILPDTPNSLIERGYTEEAKEMLQSIRGTNEVDEEFQDLIDASEESKQVKHPWKNIMLPRYRPQLIMTCFIPFFQQLTGINVITFYAPVLFQTLGFGSKASLLSAMVTGIIELLCTFVSVFTVDRFGRRILFLQGGIQMLVSQIAIGAMIGVKFGVAGTGNIGKSDANLIVALICIYVAGFAWSWGPLGWLVPSEISPLEIRSAAQAINVSVNMFFTFLVAQLFLTMLCHMKFGLFFFFAFFVVIMTIFIYLMLPETKNVPIEEMNRVWKAHWFWGKFIPDEAVNMGAAEMQQKSV

SEQ ID NO: 35 - protein, 523 aa, Nicotiana tabacum; identical to SEQ ID NO: 29
  MISC_FEATURE: Transporter GlcGal,Xyl

SEQ ID NO: 36 - protein, 518 aa, Medicago truncatula
  MISC_FEATURE: Glu Frc
MAGGGIPIGGGNKEYPGNLTPFVTITCIVAAMGGLIFGYDIGISGGVTSMDPFLKKFFPAVYRKKNKDKSTNQYCQYDSQTLTMFTSSLYLAALLSSLVASTITRRFGRKLSMLFGGLLFLVGALINGFANHVWMLIVGRILLGFGIGFANQPVPLYLSEMAPYKYRGALNIGFQLSITIGILVANVLNYFFAKIKGGWGWRLSLGGAMVPALIITIGSLVLPDTPNSMIERGDRDGAKAQLKRIRGIEDVDEEFNDLVAASEASMQVENPWRNLLQRKYRPQLTMAVLIPFFQQFTGINVIMFYAPVLFNSIGFKDDASLMSAVITGVVNVVATCVSIYGVDKWGRRALFLEGGAQMLICQVAVAAAIGAKFGTSGNPGNLPEWYAIVVVLFICIYVAGFAWSWGPLGWLVPSEIFPLEIRSAAQSVNVSVNMLFTFLVAQVFLIMLCHMKFGLFLFFAFFVLVMSIYVFFLLPETKGIPIEEMDRVWKSHPFWSRFVEHGDHGNGVEMGKGAPKNV

SEQ ID NO: 37 - protein, 526 aa, Vitis vinifera
  MISC_FEATURE: Hexose transporter 7
MEVGDGSFAPVGVSKQRADQYKGRLTTYVVVACLVAAVGGAIFGYDIGVSGGVTSMDTFLEKFFHTVYLKKRRAEEDHYCKYNDQGLAAFTSSLYLAGLVASIVASPITRKYGRRASIVCGGISFLIGAALNAAAVNLAMLLSGRIMLGIGIGFGDQAVPLYLSEMAPAHLRGALNMMFQLATTTGIFTANMINYGTAKLPSWGWRLSLGLAALPAILMTVGGLFLPETPNSLIERGSREKGRRVLERIRGTNEVDAEFEDIVDASELANSIKHPFRNILERRNRPQLVMAICMPAFQILNGINSILFYAPVLFQTMGFGNATLYSSALTGAVLVLSTVVSIGLVDRLGRRVLLISGGIQMVLCQVTVAIILGVKFGSNDGLSKGYSVLVVIVICLFVIAFGWSWGPLGWTVPSEIFPLETRSAGQSITVVVNLLFTFIIAQCFLSMLCSFKHGIFLFFAGWIVIMTLFVYFFLPETKGVPIEEMIFVWKKHWFWKRMVPGTPDVDDIDGLGSHSMESGGKTKLGS

SEQ ID NO: 38 - protein, 534 aa, Parachlorella kessleri
  MISC_FEATURE: H(+)hexose cotransporter
MAGGGVVVVSGRGLSTGDYRGGLTVYVVMVAFMAACGGLLLGYDNGVTGGVVSLEAFEKKFFPDVWAKKQEVHEDSPYCTYDNAKLQLFVSSLFLAGLVSCLFASWITRNWGRKVTMGIGGAFFVAGGLVNAFAQDMAMLIVGRVLLGFGVGLGSQVVPQYLSEVAPFSHRGMLNIGYQLFVTIGILIAGLVNYAVRDWENGWRLSLGPAAAPGAILFLGSLVLPESPNFLVEKGKTEKGREVLQKLCGTSEVDAEFADIVAAVEIARPITMRQSWASLFTRRYMPQLLTSFVIQFFQQFTGINAIIFYVPVLFSSLGSANSAALLNTVVVGAVNVGSTLIAVMFSDKFGRRFLLIEGGIQCCLAMLTTGVVLAIEFAKYGTDPLPKAVASGILAVICIFISGFAWSWGPMGWLIPSEIFTLETRPAGTAVAVVGNFLFSFVIGQAFVSMLCAMEYGVFLFFAGWLVIMVLCAIFLLPETKGVPIERVQALYARHWFWNRVMGPAAAEVIAEDEKRVAAASAIIKEEELSKAMK

SEQ ID NO: 39 - protein, 534 aa, Parachlorella kessleri; identical to SEQ ID NO: 20
  MISC_FEATURE: H(+)hexose cotransporter 3 [preference GlcFruManXyl]

SEQ ID NO: 40 - protein, 440 aa, Aspergillus fumigatus
  MISC_FEATURE: Phytase
MSKSCDTVDLGYQCSPATSHLWGQYSPFFSLEDELSVSSKLPKDCRITLVQVLSRHGARYPTSSKSKKYKKLVTAIQANATDFKGKFAFLKTYNYTLGADDLTPFGEQQLVNSGIKFYQRYKALARSVVPFIRASGSDRVIASGEKFIEGFQQAKLADPGATNRAAPAISVIIPESETFNNTLDHGVCTKFEASQLGDEVAANFTALFAPDIRARAEKHLPGVTLTDEDVVSLMDMCSFDTVARTSDASQLSPFCQLFTHNEWKKYNYLQSLGKYYGYGAGNPLGPAQGIGFTNELIARLTRSPVQDHTSTNSTLVSNPATFPLNATMYVDFSHDNSMVSIFFALGLYNGTEPLSRTSVESAKELDGYSASWVVPFGARAYFETMQCKSEKEPLVRALINDRVVPLHGCDVDKLGRCKLNDFVKGLSWARSGGNWGECFS

SEQ ID NO: 41 - protein, 465 aa, Aspergillus fumigatus
  MISC_FEATURE: Phytase
MVTLTFLLSAAYLLSGRVSAAPSSAGSKSCDTVDLGYQCSPATSHLWGQYSPFFSLEDELSVSSKLPKDCRITLVQVLSRHGARYPTSSKSKKYKKLVTAIQANATDFKGKFAFLKTYNYNLGADDLNPFGEQQLVNSGIKFYQRYKALARSVVPFIRASGSDRVIASGEKFIEGFQQAKLADPGATNRAAPAISVIIPESETFNNTLDHGVCTKFEASQLGDEVAANFTALFAPDIRARAEKHLPGVTLTDEDVVSLMDMCSFDTVARTSDASQLSPFCQLFTHNEWKKYNYLQSLGKYYGYGAGNPLGPAQGIGFTNELIARLTRSPVQDHTSTNSTLVSNPATFPLNATMYVDFSHDNSMVSIFFALGLYNGTEPLSRTSVESAKELDGYSASWVVPFGARAYFETMQCKSEKEPLVRALINDRVVPLLGCDVDKLGRCKLNDFVKGLSWARSGGNWGECFS

SEQ ID NO: 42 - DNA, 2078 bp, Artificial
  Synthetic Chlorella hexose transporter expression cassette with RBCS2 promoter and 3′ UTR
gccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaccaccatggcgggcggcgccattgttgccagcggcggcgccagccgttcgagcgagtaccagggcggcctgaccgcctacgttctgctcgtggcgctggttgccgcctgcggcggcatgctgctgggctacgacaacggcgttaccggcggcgttgccagcatggagcagttcgagcgcaagttcttcccggacgtgtacgagaagaagcagcagattgtcgagaccagcccgtactgcacctacgacaacccgaagctccagctgttcgtgtcgagcctgttcctggcgggcctgattagctgcattttctcggcgtggattacccgcaactggggccgcaaggcgagcatgggcattggcggcattttcttcattgccgccggtggcctggttaacgccttcgcccaggacattgccatgctgattgtgggccgcgtcctgctgggcttcggcgttggcctgggcagccaggtggtgccacagtacctgagcgaggtggcgccattcagccatcgcggcatgctcaacattggctaccagctcttcgtgaccattggcattctgattgccggcctggtgaactacggcgtgcgcaactgggacaacggttggcgcctgagcctgggcctggcggcggttccaggcctgattctgctgctcggcgccatcgttctgccggagagcccgaacttcctggtggagaagggccgcaccgaccagggccgccgcattctggagaagctgcgcggcaccagccatgttgaggcggagttcgccgacattgtggcggcggtggagattgcccgcccaattaccatgcgccagagctggcgctcgctgttcacccgccgctacatgccacagctgctgaccagcttcgtgattcagttcttccagcagttcaccggcattaacgccatcattttctacgtgccggtgctgttcagcagcctgggctcggcgtcctcggcggcgctgctgaacaccgtggttgtgggcgccgtgaacgtgggcagcaccatgattgccgtgctgctgtcggacaagttcggccgccgcttcctgctgattgagggcggcattacctgctgcctggcgatgctggcggcgggcattacgctgggcgtggagttcggccagtacggcaccgaggacctgccacatccagtgtcggcgggcgtgctggcggtgatttgcattttcattgccggcttcgcctggagctggggcccaatgggctggctgattccgagcgagattttcaccctggagacccgcccagcgggcacggcggttgccgtgatgggcaacttcctgttctcgttcgtgattggccaggccttcgtgtcgatgctgtgcgcgatgaagttcggcgtgttcctgttcttcgccggctggctggtgattatggtgctgtgcgccattttcctgctgccggagaccaagggcgtgccgattgagcgcgtgcaggcgctgtacgcccgccactggttctggaagaaggtgatgggcccagcggcccaggagattattgccgaggacgagaagcgcgttgcggcgagccaggcgattatgaaggaggagcgcattagccagaccatgaagtaaccgacgtcgacccactctagaggatcgatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttg

SEQ ID NO: 43 - DNA, 208 bp, Chlamydomonas reinhardtii
  misc_feature: RBCS2 promoter
gccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaa

SEQ ID NO: 44 - DNA, 1759 bp, Porphyridium sp.
  misc_feature: Promoter of glycoprotein associated with Porphyridium sp.polysaccharide
cgtcattagcgaagcgtccgcgttcacagcgttcgatggaggtggcagggacaatgatggtggagcctcttcgcacctcagcgcggcatacaccacaaaaccaccgtctaactcgagggacagcgcctggcgcacgttggatttcagggtcgcgtaccgcgccaggtcaaagtggatcttaccgaccagtttttcgttgtcactgctagccgcggtcaccgactttgccgagtctgtgtggtacagctgcagcacggcaaagcgctggtggcgatcttcaaagtttcggtccgatgcgcgtctcgacaggcgcgcaggaacgctgaaggtctcgttccacacggccacgttgggctgatggtcgaaactgacgaatgcgcttggttttcgtcccgggccgtccgagcacctgctggtctccatacgcgagtcgaaagcgatacgtgccatccactttcatgcgggtcactttctccacttcgagcgtaaatatgaacgtatgcgagcagaggtgcccaccgccactcacgttcgtgccggtgtcgacgccattcgtcgcccatgacgaggaagagagcaagacgacgtttccagccctgctcgccgacttgctctgcgccgacgacgcggacgcctgcgtcacactggaggagtttgaggcgttggacgcgcgcacgctggactctcgcttcactagcaagccactcccgctgcgatcgagcgacggcgagcgcgagcccttcctactctcactgcgcgacgaggcggtcgagtccgagcgactcacctggcttaagcgtctctcgctcatgacgcgcttgacgagcaccttgcgagtagccagccgcacctctacccgcgcatgccaacctcactccagtgtctcgcaagcaaccacgatgctccttcccagtcgaatgcgacacgcgaccacgttcgccgcgcaacttttttttccaccgcagaaattggcgcggacgaagtacagctgacccgcaccaacggtacagtcatgagcttgtaactgccacttcgcacttttgtaacgtataaaataacgatacaaaggtggattaattatttcattttttcatgttccagatgtttcaagctctcatgctttgtgagtgtttttggatgtgtagccggcaaccctacttgttcgggcaacgaaacagattgttcgtgcggttggcgcggttgacgcggttgatgacaactggatgacgaaaaccagtgcgcaagaagggggggggggttaagtatagtgtgtttagtcaacgaacggcgtgtgaatttaaatgctttggggtgtagtcaaacgggtgtggtttacaaggcgctgccaagcagcgggagtacagcgctgtacgccgccgatttcaaaaggagacacgcgcgagcaacaaaatcacgaatcgggtggattttgcgtggtgcgttcgttggggtgtgtgtgtggcgctggcggttggacacgcgtaacaaatgcaggctgttttactataaagtcgggcgagaagcggatggctggtgcggtgtgatgggcgcgcacgacgccacgcagtgcctctatgcaatcgagagggagatgcgaaaaaaagggggcggtctggtataaagtgcgcgggcacgtcgtaggtacttaaatgctgtggggacggtgaaaagagtgcgtgagtgaggtgtgtggacgaaagggagagggaagagggagtgggtggccgctgtagagaacacggtcgggtgcagtaacgaa

SEQ ID NO: 45 - DNA, 984 bp, Cauliflower mosaic virus
  misc_feature: Promoter
ccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 46 - protein, 672 aa, Cyanidioschyzon merolae
  MISC_FEATURE: Na+/glucose co-transporter
MASTAPANTAVALNTLDWVLVIVYFSALVLAIWLSTRKSSRAGSGTQSKPASEMFFLAGRSTTFFAVGASLFMSNIGSEHFIALAAAGATSGLAVASFEWMASIFVGVVLGRVFAPFYLRNSLHTVPKFLELRYAAGARRYHALATIMMAILTKVSATLYSGAIILRVLLGWPVWFSLIMILVLTTLYTSLGGLRAVIWTEVLQAFVLLAGGLALAVRSLQAVGSLAGLSELLAAQNRRQMLDLLQWPSSTTPWVEYPWPGIIFGLPALEVFYWCTDQVVVQRVLSAKSEAHARGGSLLCGFLKTLVPFMMVIPGLCAFLLFPEVAANPNQAYPTAVARLLPHGLLGLMVSAMLAALMSSLASTFNSTSTVVVYDFVIECCGLSRLSDKTLVLLGRIANIVLCAFSLAWIPIVEGMGEELYFYIQSVISYIAPPIAVVFVAGIAWRRATATGALCTLLVGGALGLVRFVVEVALRLAHREAPLGALGKIFFQSNFLYFAIFSWVFSSLLLVTVSLFTEPPSEQQLGLLFQEAGASGSHVRTTAGNQIGEASSKQVVGASRYVADEPSSSDPAARQQTVELEIESFSGTENSDEAFSVDPEHTAPSMKRVASRDTLLAGEAASQEPLFSPQGEFSAAQETFSSAAPSRLTSAALDVLSVVLVAEILAFYIQFR

SEQ ID NO: 47 - protein, 541 aa, Cyanidioschyzon merolae arabinose permease
METVTVRWKRFLSTFGARPCLLSCLLGGLLFGYHLAVFSTVTTFRSFQDWFGSWPSGEQVLLGSYFVGAFVGCLYQRVLPFLAHGAGTAAWRRYLWLRWSSVFFCLGSGLPFLIKRERMLTGRFQMGAFLVLLIHRLLIGIGAGIVNVLGPALCLEVAPSTSRGAFVFLYQLAITIGILMANLVNLATGHEDVHRQVDPVAGGAGIDMRGNFLRPLRYPLVPAILMCIGLLRYQSSMGVSAYRDATSADMLETGKPMSQRKEREHSMVASARALRPDAAEAGHSFNVISRPSLQNESLSLKDVESFAMYAVLARGSSPTARSRASVWLLLRDPRIQICMILQLLQQLTGINVVLVYGVQILEQVQSSAMGSSRRLARLSGPLYGAVLLSVMNVIATLVAVGIIDRTCRRKLYLFSTPVLAACHLALARATRAENGSSSVAFTGFLALMLFVAVFAVSHGPLAVLVANELFSPEARASANSIGMVVNAVATTAVSIGFPLLQRELFGIAGTFLFFALILMGGEYWLWRYLPETRQPTSADSS

SEQ ID NO: 48 - protein, 640 aa, Galdieria sulphuraria
  MISC_FEATURE: Myo-inositol transporter
MVEKSSDPEVPSLSHHESSISIEKQGDAATAREWAQDVNSTTTNTKLKNPLAGLTREQLLNDVEAFAKEKDLEHILDDLRKGALVAQDPREFEQMDALTESEKELLRREKTHRWSQPFMMYFMTSESSRYPPTEFGFNPACQSSVLDLLSCREWIRLLSTVRRSMYSSITHLSYAKQSRFYFAEFNVTDTWMQGLLNGAPYLCSAVIGCWTTAPLNRWFGRRGCIFISCFISFASSFWMAAAHTWWNLLLGRFLLGFAVGAKSTTTPVYGAECSPANIRGALVMMWQMWTAFGIMLGYIASVAFMDVTHPTIPGFNWRLMLGSTAIPPFFVCIQVYFCPESPRWYMMRNRYHDAYKALCKFRPSTFQAARDLYYIHAALKVEEKLREGKHLFREMFTIPRNRRAAQSSFFVMFMQQFCGVNAIMYYSSSMFREAGFDTRMALITSLGCGITNWIFALPAVYTIDTFGRRNLLLTTFPLMCIFLLFTGFSFYIPDQTSRTACVATGIYLFMIVYSPGEGPVPFTYSAEAFPLYIRDIGMSFATATTWGFNFIVSLTWPSLNKSFTPTGAFGWYAAWNFFGWIFCYFCLPETKALSLEELDQVFSVPTTKHVNHYRAMLPWYVKKYLLRRDVPPQNQLYDYY

SEQ ID NO: 49 - DNA, 20 bp, Artificial
  Synthetic inverse PCR primer #1
ggatgaactgcgcgagcgcg

SEQ ID NO: 50 - DNA, 20 bp, Artificial
  Synthetic inverse PCR primer #2
ctcggccggcggaaatagcc

SEQ ID NO: 51 - DNA, 20 bp, Artificial
  Synthetic inverse PCR primer #3
cagccataacggccaccgcc

SEQ ID NO: 52 - DNA, 21 bp, Artificial
  Synthetic inverse PCR primer #4
acggaatcacgggaacagcag

SEQ ID NO: 53 - DNA, 16 bp, Artificial
  Synthetic M13 forward primer
gttgggtaacgccagg

SEQ ID NO: 54 - DNA, 17 bp, Artificial
  Synthetic M13 reverse primer
ttcacacaggaaacagc

SEQ ID NO: 55 - DNA, 828 bp, Porphyra sp.
  misc_feature: Synthetic EF1 promoter
aagcttcgctgccaggctctccatcagcgacttgcggtcggtgctgtttggggaccggcgggaagcgcaccagaatgtggggggagacaggcagggctcagagacacgagtggagagcattgatcagtaacagtcgcacgtcagactgggtctgcgtggggccacgaaagcgcaaaaatgggcctagcaggcgccgtcaaccttctcaagctcagagcgccgccaggctgcacacccccgaggcgacggccagtagcacctgcgccacggagacaccgtctcctgctaacatacggttgttcgtccgccatcgagaacagctaggtacagaataccacgaggaagggaagaaaagccttgaatggcaaagggtggtcgaaggcaagccagagagggacgttctacatgcaccaaagccagtaggagcgcactacgcggtaagcggtcgccgaaagagggggggcgacccacgctcgtcactgcaggttgtattgtgccggtccgcaactgccactggcggtgtttgtcaagcggccaccacgagtctcgagggctcagcaattgcaccgctggcacgcagaatgtggccttccccgagtttacagtggtccttttccccgaaagcatccctaaaaaaaggaattgagcgacggcgacgccgaccgacgtccgcggcggcggccacaggcgagggacggaggaccaccggccttttaaatgggcgtcgaagaccacgtgctcggcacaccaccccgtcgacatcgctatcgtacccgtcttccccgccgattgacgtcttgagtttacctgttcacctagcctttcatc

SEQ ID NO: 56 - DNA, 4080 bp, Artificial
  Synthetic Glp promoter - glp-SOD fusion- CMV 3′UTR
cgtcattagcgaagcgtccgcgttcacagcgttcgatggaggtggcagggacaatgatggtggagcctcttcgcacctcagcgcggcatacaccacaaaaccaccgtctaactcgagggacagcgcctggcgcacgttggatttcagggtcgcgtaccgcgccaggtcaaagtggatcttaccgaccagtttttcgttgtcactgctagccgcggtcaccgactttgccgagtctgtgtggtacagctgcagcacggcaaagcgctggtggcgatcttcaaagtttcggtccgatgcgcgtctcgacaggcgcgcaggaacgctgaaggtctcgttccacacggccacgttgggctgatggtcgaaactgacgaatgcgcttggttttcgtcccgggccgtccgagcacctgctggtctccatacgcgagtcgaaagcgatacgtgccatccactttcatgcgggtcactttctccacttcgagcgtaaatatgaacgtatgcgagcagaggtgcccaccgccactcacgttcgtgccggtgtcgacgccattcgtcgcccatgacgaggaagagagcaagacgacgtttccagccctgctcgccgacttgctctgcgccgacgacgcggacgcctgcgtcacactggaggagtttgaggcgttggacgcgcgcacgctggactctcgcttcactagcaagccactcccgctgcgatcgagcgacggcgagcgcgagcccttcctactctcactgcgcgacgaggcggtcgagtccgagcgactcacctggcttaagcgtctctcgctcatgacgcgcttgacgagcaccttgcgagtagccagccgcacctctacccgcgcatgccaacctcactccagtgtctcgcaagcaaccacgatgctccttcccagtcgaatgcgacacgcgaccacgttcgccgcgcaacttttttttccaccgcagaaattggcgcggacgaagtacagctgacccgcaccaacggtacagtcatgagcttgtaactgccacttcgcacttttgtaacgtataaaataacgatacaaaggtggattaattatttcattttttcatgttccagatgtttcaagctctcatgctttgtgagtgtttttggatgtgtagccggcaaccctacttgttcgggcaacgaaacagattgttcgtgcggttggcgcggttgacgcggttgatgacaactggatgacgaaaaccagtgcgcaagaagggggggggggttaagtatagtgtgtttagtcaacgaacggcgtgtgaatttaaatgctttggggtgtagtcaaacgggtgtggtttacaaggcgctgccaagcagcgggagtacagcgctgtacgccgccgatttcaaaaggagacacgcgcgagcaacaaaatcacgaatcgggtggattttgcgtggtgcgttcgttggggtgtgtgtgtggcgctggcggttggacacgcgtaacaaatgcaggctgttttactataaagtcgggcgagaagcggatggctggtgcggtgtgatgggcgcgcacgacgccacgcagtgcctctatgcaatcgagagggagatgcgaaaaaaagggggcggtctggtataaagtgcgcgggcacgtcgtaggtacttaaatgctgtggggacggtgaaaagagtgcgtgagtgaggtgtgtggacgaaagggagagggaagagggagtgggtggccgctgtagagaacacggtcgggtgcagtaacgaaatggcccgcatggtggtggccgccgtggccgtgatggccgtgctgtcggtggccctggcccagttcattccggacgtggacattacgtggaaggtgccgatgacgctgacggtgcagaacctgtcgattttcacgggcccgaaccagttcggccgcggcattccgtcgccgtcggccattggcggcggcaacggcctggacattgtgggcggcggcggctcgctgtacatttcgccgacgggcggccaggtgcagtactcgcgcggctcgaacaacttcggcaaccaggtggccttcacgcgcgtgcgcaagaacggcaacaacgagtcggacttcgccacggtgttcgtgggcggcacgacgccgtcgttcgtgattgtgggcgactcgacggagaacgaggtgtcgttctggacgaacaacaaggtggtggtgaactcgcagggcttcattccgccgaacggcaactcggccggcggcaactcgcagtacacgttcgtgaacggcattacgggcacggccggcgccccggtgggcggcacggtgattcgccaggtgtcggcctggcgcgagattttcaacacggccggcaactgcgtgaagtcgttcggcctggtggtgcgcggcacgggcaaccagggcctggtgcagggcgtggagtacgacggctacgtggccattgactcgaacggctcgttcgccatttcgggctactcgccggccgtgaacaacgccccgggcttcggcaagaacttcgccgccgcccgcacgggcaacttcttcgccgtgtcgtcggagtcgggcgtgattgtgatgtcgattccggtggacaacgccggctgcacgctgtcgttctcggtggcctacacgattacgccgggcgccggccgcgtgtcgggcgtgtcgctggcccaggacaacgagttctacgccgccgtgggcattccgggcgccggcccgggcgaggtgcgcatttaccgcctggacggcggcggcgccacgacgctggtgcagacgctgtcgccgccggacgacattccggagctgccgattgtggccaaccagcgcttcggcgagatggtgcgcttcggcgccaactcggagacgaactacgtggccgtgggctcgccgggctacgccgccgagggcctggccctgttctacacggccgagccgggcctgacgccgaacgacccggacgagggcctgctgacgctgctggcctactcgaactcgtcggagattccggccaacggcggcctgggcgagttcatgacggcctcgaactgccgccagttcgtgttcggcgagccgtcggtggactcggtggtgacgttcctggcctcgattggcgcctactacgaggactactgcacgtgcgagcgcgagaacattttcgaccagggcattatgttcccggtgccgaacttcccgggcgagtcgccgacgacgtgccgctcgtcgatttacgagttccgcttcaactgcctgatggagggcgccccgtcgatttgcacgtactcggagcgcccgacgtacgagtggacggaggaggtggtggacccggacaacacgccgtgcgagctggtgtcgcgcattcagcgccgcctgtcgcagtcgaactgcttccaggactacgtgacgctgcaggtggtgggcgccggcgccggcatggccacgaaggccgtgtgcgtgctgaagggcgacggcccggtgcagggcattattaacttcgagcagaaggagtcgaacggcccggtgaaggtgtggggctcgattaagggcctgacggagggcctgcacggcttccacgtgcacgagttcggcgacaacacggccggctgcacgtcggccggcccgcacttcaacccgctgtcgcgcaagcacggcggcccgaaggacgaggagcgccacgtgggcgacctgggcaacgtgacggccgacaaggacggcgtggccgacgtgtcgattgaggactcggtgatttcgctgtcgggcgaccactgcattattggccgcacgctggtggtgcacgagaaggccgacgacctgggcaagggcggcaacgaggagtcgacgaagacgggcaacgccggctcgcgcctggcctgcggcgtgattggcattgcccagtaatttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 57 - DNA, 1764 bp, Artificial
  Synthetic AHAS W492S mutant cDNA
atgacacatatagaaaaatcaaattatcaagaacaaaccggggcttttgctttattagatagcttagtaagacacaaagttaaacatatttttggttaccctggcggagctatattgcctatatatgatgaattatataaatgggaagagcaaggatatataaagcatatattggttagacacgaacaaggtgctgcacatgctgcagatggttatgcaagagctaccggtgaagtaggtgtttgtttcgcaacttctggaccaggagccactaatttagtaacagggattgcaacagctcatatggattccatacctattgttataattactggccaggtaggtcgttctttcataggcactgatgcatttcaagaagttgatatttttggtattacgttaccaatagtaaaacactcgtatgtaattagggatcctcgagatatacctagaatagttgctgaagccttctctatagctaagcaggggaggcccggtccagttttaattgatgtgcctaaagatgtcggtcttgaaacattcgaatatcaatatgttaatcctggtgaagcaagaataccaggatttcgcgatctagttgctcccagctctagacaaatcattcattcaattcagttgatacaggaagctaatcagcctttactatacgtaggagggggtgcaataacttctggagctcatgacttaatttataaattagttaatcaatataaaatacctataactactacattaatgggtaaaggaattatcgatgaacaaaatcctttagctttgggtatgctaggcatgcatggtactgcttatgcaaattttgccgttagcgaatgtgatttattaataaccttaggagctcgattcgatgatagagtaacgggtaagctagatgaatttgcttgtaatgctaaagtgattcatgtcgatattgatcctgcagaagtaggaaaaaatagaattcctcaagttgccattgttggtgatattagtttagttttagaacaatggctattatatttagatagaaacttgcaattagatgatagccacttgcgttcatggcatgagcgtatatttaggtggcgccaagaatatccattaattgtacctaaattggttcaaacactgtcaccacaagaaattattgccaatattagtcaaattatgccagatgcatatttttctacagatgttggacagcatcaaatgtgggcagctcaatttgtcaaaacattacctcgaagatggctttctagttctggcttaggcacaatgggatatggcttacctgcagctataggcgccaaaatagcatatcctgaatcacctgtagtatgtattaccggtgattctagttttcaaatgaatattcaggagctaggtactatagcccaatataaattagacatcaaaattattattattaataataaatggcaaggcatggtaagacaatcgcaacaagctttttacggagcacgttattctcattcacggatggaagatggagcacctaactttgtcgcattggctaaatctttcggtattgatggtcaatctataagtactaggcaagaaatggattctttatttaatactattataaagtacaaaggtcccatggtcatagactgtaaagtaatagaagatgaaaactgttatcctatggtagctcctggcaaaagcaacgcacagatgattggcttggataaatctaataatgaaataataaaaataaaagaataa

SEQ ID NO: 58 - DNA, 543 bp, Artificial; identical to SEQ ID NO: 7
  Synthetic AHAS promoter

SEQ ID NO: 59 - DNA, 31 bp, Artificial
  Synthetic AHAS 3′ UTR
ttaagagaattattcttttatttttattata

SEQ ID NO: 60 - DNA, 1764 bp, Artificial
  Synthetic AHAS cDNA 9chloroplast- can be used to determinepreferred and most preferred codons for Porphyridium sp. chloroplast)
atgacacatatagaaaaatcaaattatcaagaacaaaccggggcttttgctttattagatagcttagtaagacacaaagttaaacatatttttggttaccctggcggagctatattgcctatatatgatgaattatataaatgggaagagcaaggatatataaagcatatattggttagacacgaacaaggtgctgcacatgctgcagatggttatgcaagagctaccggtgaagtaggtgtttgtttcgcaacttctggaccaggagccactaatttagtaacagggattgcaacagctcatatggattccatacctattgttataattactggccaggtaggtcgttctttcataggcactgatgcatttcaagaagttgatatttttggtattacgttaccaatagtaaaacactcgtatgtaattagggatcctcgagatatacctagaatagttgctgaagccttctctatagctaagcaggggaggcccggtccagttttaattgatgtgcctaaagatgtcggtcttgaaacattcgaatatcaatatgttaatcctggtgaagcaagaataccaggatttcgcgatctagttgctcccagctctagacaaatcattcattcaattcagttgatacaggaagctaatcagcctttactatacgtaggagggggtgcaataacttctggagctcatgacttaatttataaattagttaatcaatataaaatacctataactactacattaatgggtaaaggaattatcgatgaacaaaatcctttagctttgggtatgctaggcatgcatggtactgcttatgcaaattttgccgttagcgaatgtgatttattaataaccttaggagctcgattcgatgatagagtaacgggtaagctagatgaatttgcttgtaatgctaaagtgattcatgtcgatattgatcctgcagaagtaggaaaaaatagaattcctcaagttgccattgttggtgatattagtttagttttagaacaatggctattatatttagatagaaacttgcaattagatgatagccacttgcgttcatggcatgagcgtatatttaggtggcgccaagaatatccattaattgtacctaaattggttcaaacactgtcaccacaagaaattattgccaatattagtcaaattatgccagatgcatatttttctacagatgttggacagcatcaaatgtgggcagctcaatttgtcaaaacattacctcgaagatggctttctagttctggcttaggcacaatgggatatggcttacctgcagctataggcgccaaaatagcatatcctgaatcacctgtagtatgtattaccggtgattctagttttcaaatgaatattcaggagctaggtactatagcccaatataaattagacatcaaaattattattattaataataaatggcaaggcatggtaagacaatggcaacaagctttttacggagcacgttattctcattcacggatggaagatggagcacctaactttgtcgcattggctaaatctttcggtattgatggtcaatctataagtactaggcaagaaatggattctttatttaatactattataaagtacaaaggtcccatggtcatagactgtaaagtaatagaagatgaaaactgttatcctatggtagctcctggcaaaagcaacgcacagatgattggcttggataaatctaataatgaaataataaaaataaaagaataa

SEQ ID NO: 61 - DNA, 15 bp, Artificial
  Synthetic W492S-containing segment of AHAS gene
agacaatcgcaacaa

SEQ ID NO: 62 - DNA, 27 bp, Artificial
  Ble-FWD synthetic primer
gccaagttgaccagtgccgttccggtg

SEQ ID NO: 63 - DNA, 26 bp, Artificial
  Ble-REV synthetic primer
cggctgctcgccgatctcggtcatgg

SEQ ID NO: 64 - DNA, 17 bp, Artificial
  GLP-FWD synthetic primer
atacgcgagtcgaaagc

SEQ ID NO: 65 - DNA, 16 bp, Artificial
  GLP-FWD synthetic primer
acagcgctgtactccc

SEQ ID NO: 66 - DNA, 23 bp, Artificial
  GLUT1-FWD synthetic primer
tgctgatgatgaacctgctggcc

SEQ ID NO: 67 - DNA, 22 bp, Artificial
  GLUT1-REV synthetic primer
agcacaatgcactggagcagcg

SEQ ID NO: 68 - DNA, 3549 bp, Artificial
  Synthetic Glp promoter - chlorela hexose transporter- CMV 3′UTR
cgtcattagcgaagcgtccgcgttcacagcgttcgatggaggtggcagggacaatgatggtggagcctcttcgcacctcagcgcggcatacaccacaaaaccaccgtctaactcgagggacagcgcctggcgcacgttggatttcagggtcgcgtaccgcgccaggtcaaagtggatcttaccgaccagtttttcgttgtcactgctagccgcggtcaccgactttgccgagtctgtgtggtacagctgcagcacggcaaagcgctggtggcgatcttcaaagtttcggtccgatgcgcgtctcgacaggcgcgcaggaacgctgaaggtctcgttccacacggccacgttgggctgatggtcgaaactgacgaatgcgcttggttttcgtcccgggccgtccgagcacctgctggtctccatacgcgagtcgaaagcgatacgtgccatccactttcatgcgggtcactttctccacttcgagcgtaaatatgaacgtatgcgagcagaggtgcccaccgccactcacgttcgtgccggtgtcgacgccattcgtcgcccatgacgaggaagagagcaagacgacgtttccagccctgctcgccgacttgctctgcgccgacgacgcggacgcctgcgtcacactggaggagtttgaggcgttggacgcgcgcacgctggactctcgcttcactagcaagccactcccgctgcgatcgagcgacggcgagcgcgagcccttcctactctcactgcgcgacgaggcggtcgagtccgagcgactcacctggcttaagcgtctctcgctcatgacgcgcttgacgagcaccttgcgagtagccagccgcacctctacccgcgcatgccaacctcactccagtgtctcgcaagcaaccacgatgctccttcccagtcgaatgcgacacgcgaccacgttcgccgcgcaacttttttttccaccgcagaaattggcgcggacgaagtacagctgacccgcaccaacggtacagtcatgagcttgtaactgccacttcgcacttttgtaacgtataaaataacgatacaaaggtggattaattatttcattttttcatgttccagatgtttcaagctctcatgctttgtgagtgtttttggatgtgtagccggcaaccctacttgttcgggcaacgaaacagattgttcgtgcggttggcgcggttgacgcggttgatgacaactggatgacgaaaaccagtgcgcaagaagggggggggggttaagtatagtgtgtttagtcaacgaacggcgtgtgaatttaaatgctttggggtgtagtcaaacgggtgtggtttacaaggcgctgccaagcagcgggagtacagcgctgtacgccgccgatttcaaaaggagacacgcgcgagcaacaaaatcacgaatcgggtggattttgcgtggtgcgttcgttggggtgtgtgtgtggcgctggcggttggacacgcgtaacaaatgcaggctgttttactataaagtcgggcgagaagcggatggctggtgcggtgtgatgggcgcgcacgacgccacgcagtgcctctatgcaatcgagagggagatgcgaaaaaaagggggcggtctggtataaagtgcgcgggcacgtcgtaggtacttaaatgctgtggggacggtgaaaagagtgcgtgagtgaggtgtgtggacgaaagggagagggaagagggagtgggtggccgctgtagagaacacggtcgggtgcagtaacgaaatggcgggcggcgccattgttgccagcggcggcgccagccgttcgagcgagtaccagggcggcctgaccgcctacgttctgctcgtggcgctggttgccgcctgcggcggcatgctgctgggctacgacaacggcgttaccggcggcgttgccagcatggagcagttcgagcgcaagttcttcccggacgtgtacgagaagaagcagcagattgtcgagaccagcccgtactgcacctacgacaacccgaagctccagctgttcgtgtcgagcctgttcctggcgggcctgattagctgcattttctcggcgtggattacccgcaactggggccgcaaggcgagcatgggcattggcggcattttcttcattgccgccggtggcctggttaacgccttcgcccaggacattgccatgctgattgtgggccgcgtcctgctgggcttcggcgttggcctgggcagccaggtggtgccacagtacctgagcgaggtggcgccattcagccatcgcggcatgctcaacattggctaccagctcttcgtgaccattggcattctgattgccggcctggtgaactacggcgtgcgcaactgggacaacggttggcgcctgagcctgggcctggcggcggttccaggcctgattctgctgctcggcgccatcgttctgccggagagcccgaacttcctggtggagaagggccgcaccgaccagggccgccgcattctggagaagctgcgcggcaccagccatgttgaggcggagttcgccgacattgtggcggcggtggagattgcccgcccaattaccatgcgccagagctggcgctcgctgttcacccgccgctacatgccacagctgctgaccagcttcgtgattcagttcttccagcagttcaccggcattaacgccatcattttctacgtgccggtgctgttcagcagcctgggctcggcgtcctcggcggcgctgctgaacaccgtggttgtgggcgccgtgaacgtgggcagcaccatgattgccgtgctgctgtcggacaagttcggccgccgcttcctgctgattgagggcggcattacctgctgcctggcgatgctggcggcgggcattacgctgggcgtggagttcggccagtacggcaccgaggacctgccacatccagtgtcggcgggcgtgctggcggtgatttgcattttcattgccggcttcgcctggagctggggcccaatgggctggctgattccgagcgagattttcaccctggagacccgcccagcgggcacggcggttgccgtgatgggcaacttcctgttctcgttcgtgattggccaggccttcgtgtcgatgctgtgcgcgatgaagttcggcgtgttcctgttcttcgccggctggctggtgattatggtgctgtgcgccattttcctgctgccggagaccaagggcgtgccgattgagcgcgtgcaggcgctgtacgcccgccactggttctggaagaaggtgatgggcccagcggcccaggagattattgccgaggacgagaagcgcgttgcggcgagccaggcgattatgaaggaggagcgcattagccagaccatgaagtaatttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 69 - DNA, 185 bp, Cauliflower mosaic virus
  misc_feature: 3′ UTR
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 70 - DNA, 408 bp, Artificial
  Synthetic zeocin resistance ble cDNA
atacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacggacc

SEQ ID NO: 71 - protein, 485 aa, Artificial
  Synthetic hexokinase
MVHLGPKKPQARKGSMADVPKELMDEIHQLEDMFTVDSETLRKVVKHFIDELNKGLTKKGGNIPMIPGWVMEFPTGKESGNYLAIDLGGTNLRVVLVKLSGNHTFDTTQSKYKLPHDMRTTKHQEELWSFIADSLKDFMVEQELLNTKDTLPLGFTFSYPASQNKINEGILQRWTKGFDIPNVEGHDVVPLLQNEISKRELPIEIVALINDTVGTLIASYYTDPETKMGVIFGTGVNGAFYDVVSDIEKLEGKLADDIPSNSPMAINCEYGSFDNEHLVLPRTKYDVAVDEQSPRPGQQAFEKMTSGYYLGELLRLVLLELNEKGLMLKDQDLSKLKQPYIMDTSYPARIEDDPFENLEDTDDIFQKDFGVKTTLPERKLIRRLCELIGTRAARLAVCGIAAICQKRGYKTGHIAADGSVYNKYPGFKEAAAKGLRDIYGWTGDASKDPITIVPAEDGSGAGAAVIAALSEKRIAEGKSLGIIGA

(19) United States
(12) Patent Application Publication
(10) Pub. No.: US 2009/0274736 A1
(43) Pub. Date: Nov. 5, 2009

(54) Nutraceutical Compositions From Microalgae And Related Methods of Production And Administration

(75) Inventors: Harrison F. Dillon, South San Francisco, CA (US); Aravind Somanchi, South San Francisco, CA (US); Anthony G. Day, South San Francisco, CA (US); Anwar Zaman, South San Francisco, CA (US); Jonathan Wolfson, South San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Solazyme Inc., South San Francisco, CA (US)

(21) Appl. No.: 11/932,754

(22) Filed: Oct. 31, 2007

Related U.S. Application Data
(63) Continuation of application No. PCT/US2007/001319, filed on Jan. 19, 2007.
Application No. PCT/US2007/001319 is a continuation-in-part of application No. 11/336,426, filed on Jan. 19, 2006.
Application No. 11/336,426 is a continuation-in-part of application No. 11/337,103, filed on Jan. 19, 2006.
Application No. 11/337,103 is a continuation-in-part of application No. 11/336,656, filed on Jan. 19, 2006, now abandoned.
Application No. 11/336,656 is a continuation-in-part of application No. 11/336,428, filed on Jan. 19, 2006, now abandoned.
Application No. 11/336,428 is a continuation-in-part of application No. 11/337,171, filed on Jan. 19, 2006, now abandoned.
Application No. 11/337,171 is a continuation-in-part of application No. 11/336,431, filed on Jan. 19, 2006, now abandoned.
Application No. 11/336,431 is a continuation-in-part of application No. 11/336,430, filed on Jan. 19, 2006, now abandoned.
(60) Provisional application No. 60/816,967, filed on Jun. 28, 2006.
(60) Provisional application No. 60/832,091, filed on Jul. 20, 2006.
(60) Provisional application No. 60/838,452, filed on Aug. 17, 2006.
(60) Provisional application No. 60/872,072, filed on Nov. 30, 2006.

(51) Int. Cl. A61P 9/00; A61K 36/02; A61K 38/44; A61P 19/02

(52) U.S. Cl. 424/422; 424/195.17; 424/94.4

(57) ABSTRACT

Provided herein are polysaccharide compositions and methods of culturing microalgae to produce polysaccharides. Also provided are methods of using polysaccharides for applications such as reducing cholesterol in mammals, inactivating viruses, stabilizing foods, and other uses. Also provided are transgenic algae capable of utilizing fixed carbon sources for energy. Also provided herein are novel nucleic acid sequences from red microalgae.

### BACKGROUND OF THE INVENTION

Carbohydrates have the general molecular formula CH₂O, and thus were once thought to represent “hydrated carbon”. However, the arrangement of atoms in carbohydrates has little to do with water molecules. Starch and cellulose are two common carbohydrates. Both are macromolecules with molecular weights in the hundreds of thousands. Both are polymers; that is, each is built from repeating units, monomers, much as a chain is built from its links.

Three common sugars share the same molecular formula: C₆H₁₂O₆. Because of their six carbon atoms, each is a hexose. Glucose is the immediate source of energy for cellular respiration. Galactose is a sugar in milk. Fructose is a sugar found in honey. Although all three share the same molecular formula (C₆H₁₂O₆), the arrangement of atoms differs in each case. Substances such as these three, which have identical molecular formulas but different structural formulas, are known as structural isomers. Glucose, galactose, and fructose are “single” sugars or monosaccharides.

Two monosaccharides can be linked together to form a “double” sugar or disaccharide. Three common disaccharides are sucrose, common table sugar (glucose+fructose); lactose, the major sugar in milk (glucose+galactose); and maltose, the product of starch digestion (glucose+glucose). Although the process of linking the two monomers is complex, the end result in each case is the loss of a hydrogen atom (H) from one of the monosaccharides and a hydroxyl group (OH) from the other. The resulting linkage between the sugars is called a glycosidic bond. The molecular formula of each of these disaccharides is C₁₂H₂₂O₁₁=2 C₆H₁₂O₆—H₂O. All sugars are very soluble in water because of their many hydroxyl groups. Although not as concentrated a fuel as fats, sugars are the most important source of energy for many cells.

### BRIEF SUMMARY OF THE INVENTION

The present invention relates to polysaccharides from microalgae. Representative polysaccharides include those present in the cell wall of microalgae as well as secreted polysaccharides, or exopolysaccharides. In addition to the polysaccharides themselves, such as in an isolated, purified, or semi-purified form, the invention includes a variety of compositions containing one or more microalgal polysaccharides as disclosed herein. The compositions include nutraceutical, cosmeceutical, industrial and pharmaceutical compositions which may be used for a variety of indications and uses as described herein. Other compositions include those containing one or more microalgal polysaccharides and a suitable carrier or excipient for topical or oral administration.

The invention further relates to methods of producing or preparing microalgal polysaccharides. In some disclosed methods, exogenous sugars are incorporated into the polysaccharides to produce polysaccharides distinct from those present in microalgae that do not incorporate exogenous sugars. The invention also includes methods of trophic conversion and recombinant gene expression in microalgae. In some methods, recombinant microalgae are prepared to express heterologous gene products, such as mammalian proteins as a non-limiting example, while in other embodiments, the microalgae are modified to produce more of a small molecule already made by microalgae in the absence of genetic modification.

Additionally, the invention relates to methods of using the polysaccharides and/or compositions containing them. In some disclosed methods, one or more polysaccharides are used to lower cholesterol, prevent sexually transmitted diseases, lubricate joints, regulate insulin levels, enhance cosmetics, stabilize or emulsify foods, and treat or effect prophylaxis of inflammation.

So in one aspect, the invention includes a nutraceutical composition containing one or more polysaccharides disclosed herein and a carrier suitable for human consumption. In other aspects, the composition contains the carrier and homogenized microalgae cells, such as red microalgae cells as a non-limiting example. In some embodiments, the composition contains the carrier and a purified first polysaccharide produced from a microalgal species listed in Table 1, which lists non-limiting examples of microalgae for the practice of the invention. Non-limiting examples of the carrier include a human nutritional supplement, such as vitamins, minerals, herbal extracts, monosaccharides or polysaccharides (e.g. glucosamine, glucosamine sulfate, chondroitin, or chondroitin sulfate, etc.) and proteins (e.g. protein supplements, etc.); a human food product; and various human foods per se.

In another aspect, the invention relates to compositions for topical application. In some embodiments, the composition is that of a cosmeceutical. A cosmeceutical may contain one or more microalgal polysaccharides, or a microalgal cell homogenate, and a topical carrier. In some embodiments, the carrier may be any carrier suitable for topical application, such as, but not limited to, use on human skin or human mucosal tissue. In some embodiments, the composition may contain a purified microalgal polysaccharide, such as an exopolysaccharide, and a topical carrier.

As a cosmeceutical, the composition may contain a microalgal polysaccharide or homogenate and other component material found in cosmetics. In some embodiments, the component material may be that of a fragrance, a colorant (e.g. black or red iron oxide, titanium dioxide and/or zinc oxide, etc.), a sunblock (e.g. titanium, zinc, etc.), and a mineral or metallic additive.

In other aspects, the invention includes methods of preparing or producing a microalgal polysaccharide. In some aspects relating to an exopolysaccharide, the invention includes methods that separate the exopolysaccharide from other molecules present in the medium used to culture exopolysaccharide producing microalgae. In some embodiments, separation includes removal of the microalgae from the culture medium containing the exopolysaccharide, after the microalgae has been cultured for a period of time. Of course the methods may be practiced with microalgal polysaccharides other than exopolysaccharides. In other embodiments, the methods include those where the microalgae was cultured in a bioreactor, optionally where a gas is infused into the bioreactor.

In one embodiment, the invention includes a method of producing an exopolysaccharide, wherein the method comprises culturing microalgae in a bioreactor, wherein gas is infused into the bioreactor; separating the microalgae from culture media, wherein the culture media contains the exopolysaccharide; and separating the exopolysaccharide from other molecules present in the culture media.

The microalgae of the invention may be that of any species, including those listed in Table 1 herein. In some embodiments, the microalgae is a red algae, such as the red algae Porphyridium, which has two known species (Porphyridium sp. and Porphyridium cruentum) that have been observed to secrete large amounts of polysaccharide into their surrounding growth media. In other embodiments, the microalgae is of a genus selected from Rhodella, Chlorella, and Achnanthes. Non-limiting examples of species within a microalgal genus of the invention include Porphyridium sp., Porphyridium cruentum, Porphyridium purpureum, Porphyridium aerugineum, Rhodella maculata, Rhodella reticulata, Chlorella autotrophica, Chlorella stigmatophora, Chlorella capsulata, Achnanthes brevipes and Achnanthes longipes.

In some embodiments, a polysaccharide preparation method is practiced with culture media containing over 26.7, or over 27, mM sulfate (or total SO₄²⁻). Non-limiting examples include media with more than about 28, more than about 30, more than about 35, more than about 40, more than about 45, more than about 50, more than about 55, more than about 60, more than about 65, more than about 70, more than about 75, more than about 80, more than about 85, more than about 90, more than about 95, or more than about 100 mM sulfate. In additional embodiments, more than about 150, more than about 200, more than about 250, more than about 300, more than about 350, more than about 400, more than about 450, more than about 500, more than about 550, more than about 600, more than about 650, more than about 700, or more than about 750, more than about 800, more than about 850, more than about 900, more than about 950, or more than about 1 M sulfate may be used to culture the microalgae and produce polysaccharides. Sulfate in the media may be provided in one or more of the following forms: Na₂SO₄.10H₂O, MgSO₄.7H₂0, MnSO₄, and CuSO₄.

Other embodiments of the method include the separation of an exopolysaccharide from other molecules present in the culture media by tangential flow filtration. Alternatively, the methods may be practiced by separating an exopolysaccharide from other molecules present in the culture media by alcohol precipitation. Non-limiting examples of alcohols to use include ethanol, isopropanol, and methanol.

In other embodiments, a method may further comprise treating a polysaccharide or exopolysaccharide with a protease to degrade polypeptide (or proteinaceous) material attached to, or found with, the polysaccharide or exopolysaccharide. The methods may optionally comprise separating the polysaccharide or exopolysaccharide from proteins, peptides, and amino acids after protease treatment.

In addition to preparation or production of a polysaccharide per se, the invention includes methods of preparing a composition containing a microalgal polysaccharide or homogenate. In some embodiments, a method of producing a nutraceutical composition is described. As a non-limiting example, the composition may be prepared by drying a homogenate of microalgae after the microalgae have been disrupted to produce a homogenate. In some embodiments, the microalgae is separated from the culture medium used to grow the microalgae. One non-limiting example of microalgae uses red microalgae to prepare the homogenate. Thus a homogenate processed as described herein may be combined with an appropriate carrier to form a nutraceutical of the invention.

In addition to a composition comprising a homogenate of red microalgae cells, the invention further includes a composition comprising a first component comprising cell material of the genus Porphyridium and at least one cholesterol lowering agent or compound. In some embodiments, the cell material is a cholesterol lowering homogenate, or a preparation of sulfated polysaccharides, and the agent or compound is selected from a plant phytosterol, a limonoid, a flavonoid, a tocotrienol, or any combination thereof. In additional embodiments, the agent is an inhibitor of HMG CoA reductase, such as a statin. The cell material may also be cells (biomass) per se, and in further embodiments, the cell material and cholesterol lowering agent may be packaged for sale as a single unit.

A method of formulating the above described composition is also disclosed. In some embodiments, the method comprises culturing cells of the genus Porphyridium; separating the cells from culture media to prepare a cell containing material (biomass); drying the cell containing material; and mixing the dried material with at least one cholesterol lowering agent or compound. In some embodiments, the dried material is homogenized before mixing with the agent or compound. In some embodiments the cells are homogenized before drying. In representative cases, the agent or compound is selected from a plant phytosterol, a limonoid, a flavonoid, a tocotrienol, or a combination thereof, including a combination of two, three or all four thereof.

In other embodiments, a method of formulating a cosmeceutical composition is disclosed. As one non-limiting example, the composition may be prepared by adding separated polysaccharides, or exopolysaccharides, to homogenized microalgal cells before, during, or after homogenization. Both the polysaccharides and the microalgal cells may be from a culture of microalgae cells in suspension and under conditions allowing or permitting cell division. The culture medium containing the polysaccharides is then separated from the microalgal cells followed by 1) separation of the polysaccharides from other molecules in the medium and 2) homogenization of the cells.

Other compositions of the invention may be formulated by subjecting a culture of microalgal cells and soluble exopolysaccharide to tangential flow filtration until the composition is substantially free of salts. Alternatively, a polysaccharide is prepared after proteolysis of polypeptides present with the polysaccharide. The polysaccharide and any contaminating polypeptides may be that of a culture medium separated from microalgal cells in a culture thereof. In some embodiments, the cells are of the genus Porphyridium.

In further aspects, the invention relates to methods of using a composition of the invention. In one aspect, a method of lowering serum cholesterol is described. The method may include orally administering, to a subject in need thereof, a polysaccharide produced by microalgae in combination with a biologically acceptable carrier. In other embodiments, such a method is practiced by using a cholesterol lowering composition as described herein. One non-limiting example of such a composition contains a purified microalgal exopolysaccharide, or a microalgal cell homogenate, and a carrier suitable for human oral consumption.

In another embodiment, a method of preventing a sexually transmitted disease is described. In one embodiment, a method includes administration of a solution comprising a polysaccharide produced by microalgae and use of a prophylactic device. In other embodiments, the solution is administered via the device.

In a further embodiment, a method of mammalian joint lubrication is described. In one embodiment, a method includes injecting polysaccharide produced by microalgae into a cavity containing synovial fluid.

In yet another embodiment, a method of regulating insulin is described. In one embodiment, a method includes administering a polysaccharide produced by microalgae.

In an additional embodiment, a method of cosmetic enhancement is described. In one embodiment, a method may include injecting a polysaccharide produced by microalgae into mammalian skin.

In a yet further embodiment, a method of stabilizing or emulsifying a food composition is described. In one embodiment, a method includes adding a polysaccharide produced by microalgae into a food composition.

In a yet additional embodiment, a method of treating or effecting prophylaxis of mammalian inflammation is described. In one embodiment, a method includes administering a polysaccharide produced by microalgae to a mammal.

An additional aspect of the invention includes a method of reducing reactive oxygen species (ROS) in a mammal. In some embodiments, the method comprises administering a composition comprising disrupted red microalgae cells as described herein. In other embodiments, the composition comprises one or more other agents or compounds with anti-oxidant activity. In further embodiments, the method may be used to lower the level of ROS, or reduce or decrease the amount of damage caused by ROS.

A composition comprising homogenized red microalgae cells as described herein may also be used in additional methods of the invention. In one set of embodiments, the composition may be used in a method of delaying the onset of, or treating, a neurodegenerative disease in a mammal. The neurodegenerative disease may be selected from Parkinson's disease, Alzheimer's disease, amyotrophic lateral sclerosis, Freidreich's ataxia, Huntington's disease, or a Prion disease.

The composition may also be used in a method of reducing organ transplant rejection, a method of reducing the percentage of a mammalian body that is made of fat, a method of increasing satiety (or decreasing appetite) in a mammal, a method of increasing the energy expenditure of a mammal, a method of reducing the body weight of a mammal, a method of reducing inflammation in a mammal, or a method for the prevention and/or treatment of atherosclerosis. Each of these methods may comprise administering a composition comprising homogenized red microalgae cells in a sufficient or effective amount as described herein.

In further aspects, the invention describes recombinant methods to modify microalgal cells. In some embodiments, the methods produce a microalgal cell that expresses an exogenous gene product. The exogenous gene product may encode a carbohydrate transporter protein as a non-limiting example. In other embodiments, a microalgal cell containing an exogenous gene encoding a mammalian growth hormone is described. The recombinantly modified cells per se, whether newly created or maintained in culture, are also part of the invention.

The invention also describes methods of recombinantly modifying a microalgal cell. In some embodiments, a method of trophically converting a microalgal cell, such as members of the genus Porphyridium, is described. The method may include selecting cells for a phenotype after transforming cells with a nucleic acid molecule in an expressible form. In some methods, the phenotype may be the ability to undergo cell division in the absence of light and/or in the presence of a carbohydrate that is transported by a carbohydrate transporter protein encoded by the nucleic acid molecule.

These methods may also be considered a method of expressing an exogenous gene in a microalgal cell. The method may include use of an expression vector containing a nucleic acid sequence encoding a polypeptide, such as a carbohydrate transporter protein. Alternatively, the method may include transforming a microalgal cell with a dual expression vector containing 1) a resistance cassette with a gene encoding a protein that confers resistance to an antibiotic, such as zeocin as a non-limiting example, operably linked to a promoter active in microalgae; and 2) a second expression cassette with a gene encoding a second protein operably linked to a promoter active in microalgae. After transformation, cells may be selected for the ability to survive in the presence of the antibiotic, such as at least 2.5 μg/ml zeocin as a non-limiting example where zeocin resistance is used. Alternatively, the antibiotic can be at least 3.0 μg/ml zeocin, at least 4.0 μg/ml zeocin, at least 5.0 μg/ml zeocin, at least 6.0 μg/ml zeocin, at least 7.0 μg/ml zeocin, and at least 8.0 μg/ml zeocin.

The invention further relates to microalgal cells expressing a carbohydrate transporter protein for use in a method of producing a glycopolymer. In some embodiments, the method may include providing a transgenic cell containing an expressible gene encoding a monosaccharide transporter; and culturing the cell in the presence of at least one monosaccharide, transported into the cell by the transporter, wherein the monosaccharide is incorporated into a polysaccharide made by the cell.

Alternatively, a method of trophically converting a microalgae cell may include selecting for the ability to undergo cell division in the absence of light after subjecting the microalgal cell to a mutagen and placing the cell in the presence of a molecule listed in Tables 2 or 3 herein.

Another embodiment of the invention includes a recombinant nucleic acid comprising an endogenous promoter from Porphyridium sp. Also included in the invention is a recombinant promoter sequence that drives expression of the gene encoding the glycoprotein associated with the polysaccharide from Porphyridium sp., GenBank accession number AAV48590. In some embodiments the promoter or a segment thereof is operably linked to a coding region encoding a carbohydrate transporter or an antibiotic resistance gene.

The invention further comprises a cell of the genus Porphyridium, wherein the genome of the cell contains an exogenous gene encoding a carbohydrate transport protein. In some embodiments the species is selected from the group consisting of Porphyridium sp. UTEX 637 or a strain derived from Porphyridium sp. UTEX 637, Porphyridium cruentum UTEX 161 or a strain derived from Porphyridium cruentum UTEX 161, Porphyridium aerugineum or a strain derived from Porphyridium aerugineum, Porphyridium sordidum or a strain derived from Porphyridium sordidum, Porphyridium purpureum or a strain derived from Porphyridium purpureum. In some embodiments the carbohydrate transport protein has at least 60% amino acid identity with a member of the group consisting of SEQ ID NOs: 20, 22, 24, 26, 27, 29-39, and 46-48.

The details of additional embodiments of the invention are set forth in the accompanying drawings and the description below. Other features and advantages of the invention will be apparent from the drawings and detailed description, and from the claims.

### BRIEF DESCRIPTION OF THE DRAWINGS

FIG. 1 shows precipitation of 4 liters of Porphyridium cruentum exopolysaccharide using 38.5% isopropanol. (a) supernatant; (b) addition of 38.5% isopropanol; (c) precipitated polysaccharide; (d) separating step.

FIG. 2 shows Porphyridium sp. cultured on agar plates containing various concentrations of zeocin.

FIG. 3 shows growth of Porphyridium sp. and Porphyridium cruentum cells grown in light in the presence of various concentrations of glycerol.

FIG. 4 shows Porphyridium sp. cells grown in the dark in the presence of various concentrations of glycerol.

FIG. 5 shows levels of solvent-accessible polysaccharide in Porphyridium sp. homogenates subjected to various amounts of physical disruption from Sonication Experiment 1.

FIG. 6 shows levels of solvent-accessible polysaccharide in Porphyridium sp. homogenates subjected to various amounts of physical disruption from Sonication Experiment 2.

FIG. 7 shows sexually transmitted disease prevention devices containing various amounts of exopolysaccharide.

FIG. 8 shows protein concentration measurements of autoclaved, protease-treated, and diafiltered exopolysaccharide.

FIG. 9 shows various amounts and ranges of amounts of compounds found per gram of cells in cells of the genus Porphyridium.

FIG. 10 (a) shows the diets used for animals of Groups 1 to 8. FIG. 10 (b) shows total serum cholesterol (TSC) levels (in mmol/L) in mice administered the diets of Groups 1 to 8. FIG. 10 (c) shows the mean TSC levels in mice administered the diets of Groups 1 to 8.

FIG. 11 (a) shows the average food intake (grams) per day in mice administered the diets of Groups 1 to 8. FIG. 11 (b) shows the average body weight in mice administered the diets of Groups 1 to 8 over a 4 week period.

FIG. 12 shows the percentage of total fat mass in mice administered the diets of Groups 1 to 8.

FIG. 13 shows the energy expenditure in mice administered the diets of Groups 1 to 8.

FIG. 14 shows the mean concentration of plasma antioxidants in mice administered the diets of Groups 1 to 8.

FIG. 15(a) shows PCR genotyping of two Porphyridium transformants for the ble antibiotic resistance transgene. FIG. 15(b) shows PCR genotyping of two Porphyridium transformants for the endogenous glycoprotein gene promoter. FIG. 15(c) shows PCR genotyping of a selected Porphyridium transformant for an exogenous gene encoding a recoded human GLUT1 transporter.

FIG. 16 shows a Southern blot indicating chromosomal integration of an exogenous gene encoding a recoded human GLUT1 transporter.

### DETAILED DESCRIPTION OF THE INVENTION

U.S. Pat. No. 7,135,290 is hereby incorporated in its entirety for all purposes. This application is a continuation-in-part of and claims priority to U.S. patent application Ser. No. 11/336,426, filed Jan. 19, 2006, entitled “Polysaccharide Compositions and Methods of Administering, Producing, and Formulating Polysaccharide Compositions”, which is hereby incorporated in its entirety for all purposes. This application is a continuation-in-part of and claims priority to U.S. patent application Ser. No. 11/337,103, filed Jan. 19, 2006, entitled “Methods and Compositions for Improving the Health and Appearance of Skin”, which is hereby incorporated in its entirety for all purposes. This application is a continuation-in-part of and claims priority to U.S. patent application Ser. No. 11/336,656, filed Jan. 19, 2006, entitled “Devices and Solutions for Prevention of Sexually Transmitted Diseases”, which is hereby incorporated in its entirety for all purposes. This application is a continuation-in-part of and claims priority to U.S. patent application Ser. No. 11/336,428, filed Jan. 19, 2006, entitled “Methods and Compositions for Cholesterol Reduction in Mammals”, which is hereby incorporated in its entirety for all purposes. This application is a continuation-in-part of and claims priority to U.S. patent application Ser. No. 11/337,171, filed Jan. 19, 2006, entitled “Methods and Compositions for Reducing Inflammation and Preventing Oxidative Damage”, which is hereby incorporated in its entirety for all purposes. This application is a continuation-in-part of and claims priority to U.S. patent application Ser. No. 11/336,431, filed Jan. 19, 2006, entitled “Methods and Compositions for Thickening, Stabilizing and Emulsifying Foods”, which is hereby incorporated in its entirety for all purposes. This application is a continuation-in-part of and claims priority to U.S. patent application Ser. No. 11/336,430, filed Jan. 19, 2006, entitled “Methods and Compositions for Joint Lubrication”, which is hereby incorporated in its entirety for all purposes. This application is a nonprovisional of and claims priority to U.S. Patent application No. 60/832,091, filed Jul. 20, 2006, entitled “Decolorized Microalgal Compositions for Skin Care Products”, which is hereby incorporated in its entirety for all purposes. This application is a nonprovisional of and claims priority to U.S. Patent application No. 60/838,452, filed Aug. 17, 2006, entitled “Polysaccharide Compositions and Methods of Administering, Producing, and Formulating Polysaccharide Compositions”, which is hereby incorporated in its entirety for all purposes. This application is a nonprovisional of and claims priority to U.S. Patent application No. 60/816,967, filed Jun. 28, 2006, entitled “Zeaxanthin Production Methods and Novel Compositions Containing Zeaxanthin”, which is hereby incorporated in its entirety for all purposes. This application is a nonprovisional of and claims priority to U.S. Patent application No. 60/872,072, filed Nov. 30, 2006, entitled “Polysaccharide Compositions and Methods of Administering, Producing, and Formulating Polysaccharide Compositions”, which is hereby incorporated in its entirety for all purposes. U.S. patent application Ser. No. ______, filed Jan. 19, 2007, entitled “Microalgae-derived Compositions for Improving the Health and Appearance of Skin”, is hereby incorporated in its entirety for all purposes. All other references cited are incorporated in their entirety for all purposes.

### DEFINITIONS

The following definitions are intended to convey the intended meaning of terms used throughout the specification and claims, however they are not limiting in the sense that minor or trivial differences fall within their scope.

“Active in microalgae” means a nucleic acid that is functional in microalgae. For example, a promoter that has been used to drive an antibiotic resistance gene to impart antibiotic resistance to a transgenic microalgae is active in microalgae. Nonlimiting examples of promoters active in microalgae are promoters endogenous to certain algae species and promoters found in plant viruses.

“Antiviral lubricant” means a molecule that possesses both antiviral activity and lubricant activity.

“ARA” means Arachidonic acid.

“Associates with” means, within the context of a polysaccharide binding fusion protein, one molecule binding to another molecule. Affinity and selectivity of binding can vary when a polysaccharide and a polysaccharide binding protein are in association with each other.

“Axenic” means a culture of an organism that is free from contamination by other living organisms.

“Bioreactor” means an enclosure or partial enclosure in which cells are cultured in suspension.

“Carbohydrate modifying enzyme” means an enzyme that utilizes a carbohydrate as a substrate and structurally modifies the carbohydrate.

“Carbohydrate transporter” means a polypeptide that resides in a lipid bilayer and facilitates the transport of carbohydrates across the lipid bilayer.

“Carrier suitable for human consumption” refers to compounds and materials suitable for human ingestion or otherwise physiologically compatible with oral administration to humans. Usually, such carriers are of plant or animal origin. Although such carriers sometimes contain residual amounts of solvents and buffers used in the processing of the polysaccharides and other compositions of the invention, they do not consist exclusively of such solvents or buffers, and usually have less than 50% and preferably less than 10% w/w of such solvents or buffers.

“Carrier suitable for topical administration” means a compound that may be administered, together with one or more compounds of the present invention, and which does not destroy the activity thereof and is nontoxic when administered in concentrations and amounts sufficient to deliver the compound to the skin or a mucosal tissue.

“Cell material” means any matter obtained from cells. Cell material can be intact cells, disrupted cells, a purified component of cells, and extract of cells, and other materials.

“Combination Product” means a product that comprises at least two distinct compositions intended for human administration through distinct routes, such as a topical route and an oral route. In some embodiments the same active agent is contained in both the topical and oral components of the combination product.

As used herein, “comprise” or “comprising” or “include” or “including” or variants thereof are used in the “open” sense such that the terms are inclusive and permit the presence of additional elements. The terms specify the presence of the stated features, steps, or components as recited without precluding the presence or addition of one or more features, steps, or components.

“Conditions favorable to cell division” means conditions in which cells divide at least once every 72 hours.

“Derived from” means, within the context of a microalgae strain, a strain that has been generated through passaging or mutagenesis from a starting strain. For example, Porphyridium strain UTEX 637 can be subjected to chemical mutagenesis to generate new colonies. Each new colony is a strain derived from Porphyridium strain UTEX 637.

“DHA” means Docosahexaenoic acid.

“Endopolysaccharide” means a polysaccharide that is retained intracellularly.

“EPA” means eicosapentaenoic acid.

“Exogenous gene” means a gene transformed into a wild-type organism. The gene can be heterologous from a different species, or homologous from the same species, in which case the gene occupies a different location in the genome of the organism than the endogenous gene.

“Exogenously provided” describes a molecule provided to the culture media of a cell culture.

“Exopolysaccharide” means a polysaccharide that is secreted from a cell into the extracellular environment.

“Filtrate” means the portion of a tangential flow filtration sample that has passed through the filter.

“Fixed carbon source” means molecule(s) containing carbon that are present at ambient temperature and pressure in solid or liquid form.

“Glycopolymer” means a biologically produced molecule comprising at least two monosaccharides. Examples of glycopolymers include glycosylated proteins, polysaccharides, oligosaccharides, and disaccharides.

A coding sequence is described as “heterologous” when it is physically connected to a nucleic acid other than that which it is naturally connected to in a wild-type genome. A coding sequence not found in a wild-type genome is necessarily heterologous to a promoter that it is in operable linkage with.

“Homogenate” means cell biomass that has been disrupted. A homogenate is not necessarily completely homogeneous, and as used herein can refer to a composition of cells that have been disrupted to the point where a majority of the cells in the preparation have been ruptured. A homogenate may contain chunks of cell wall, intact or partially intact organelles such as chloroplasts and mitochondria, and other substructures of the cell.

“Mammal” as used herein includes animal subjects, such as primates, as well has human patients.

“Microalgae” means a single-celled organism that is capable of performing photosynthesis. Microalgae include obligate photoautotrophs, which cannot metabolize a fixed carbon source as energy, as well as heterotrophs, which can live solely off of light, solely off of a fixed carbon source, or a combination of the two.

“Naturally produced” describes a compound that is produced by a wild-type organism.

“Pharmaceutically acceptable carrier or adjuvant” refers to a carrier or adjuvant that may be administered to a patient, together with one or more compounds of the present invention, and which does not destroy the pharmacological activity thereof and is nontoxic when administered in doses sufficient to deliver a therapeutic amount of the compound.

“Photobioreactor” means a waterproof container, at least part of which is at least partially transparent, allowing light to pass through, in which one or more microalgae cells are cultured. Photobioreactors may be sealed, as in the instance of a polyethylene bag, or may be open to the environment, as in the instance of a pond.

“Polysaccharide material” is a composition that contains more than one species of polysaccharide, and optionally contaminants such as proteins, lipids, and nucleic acids, such as, for example, a microalgal cell homogenate.

“Polysaccharide” means a compound or preparation containing one or more molecules that contain at least two saccharide molecules covalently linked. A “polysaccharide”, “endopolysaccharide” or “exopolysaccharide” can be a preparation of polymer molecules that have similar or identical repeating units but different molecular weights within the population.

“Port”, in the context of a photobioreactor, means an opening in the photobioreactor that allows influx or efflux of materials such as gases, liquids, and cells. Ports are usually connected to tubing leading to and/or from the photobioreactor.

“Red microalgae” means unicellular algae that is of the list of classes comprising Bangiophyceae, Florideophyceae, Goniotrichales, or is otherwise a member of the Rhodophyta.

“Retentate” means the portion of a tangential flow filtration sample that has not passed through the filter.

“Selectively binds to” refers to a binding reaction which is determinative of the presence of a molecule in the presence of a heterogeneous population of other molecules. Thus, under designated conditions, a specified ligand binds preferentially to a particular molecule and does not bind in a significant amount to other proteins present in the sample. A molecule such as antibody that specifically binds to a protein often has an association constant of at least 10⁶ M⁻¹ or 10⁷ M⁻¹, preferably 10⁸ M⁻¹ to 10⁹ M⁻¹, and more preferably, about 10¹⁰ M⁻¹ to 10¹¹M⁻¹ or higher. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Small molecule” means a molecule having a molecular weight of less than 2000 daltons, in some instances less than 1000 daltons, and in still other instances less than 500 daltons or less. Such molecules include, for example, heterocyclic compounds, carbocyclic compounds, sterols, amino acids, lipids, carotenoids and polyunsaturated fatty acids.

A molecule is “solvent available” when the molecule is isolated to the point at which it can be dissolved in a solvent, or sufficiently dispersed in suspension in the solvent such that it can be detected in the solution or suspension. For example, a polysaccharide is “solvent available” when it is sufficiently isolated from other materials, such as those with which it is naturally associated, such that the polysaccharide can be dissolved or suspended in an aqueous buffer and detected in solution using a dimethylmethylene blue (DMMB) or phenol:sulfuric acid assay. In the case of a high molecular weight polysaccharide containing hundreds or thousands of monosaccharides, part of the polysaccharide can be “solvent available” when it is on the outermost layer of a cell wall while other parts of the same polysaccharide molecule are not “solvent available” because they are buried within the cell wall. For example, in a culture of microalgae in which polysaccharide is present in the cell wall, there is little “solvent available” polysaccharide since most of the cell wall polysaccharide is sequestered within the cell wall and not available to solvent. However, when the cells are disrupted, e.g., by sonication, the amount of “solvent available” polysaccharide increases. The amount of “solvent accessible” polysaccharide before and after homogenization can be compared by taking two aliquots of equal volume of cells from the same culture, homogenizing one aliquot, and comparing the level of polysaccharide in solvent from the two aliquots using a DMMB assay. The amount of solvent accessible polysaccharide in a homogenate of cells can also be compared with that present in a quantity of cells of the same type in a different culture needed to generate the same amount of homogenate.

“Substantially free of protein” means compositions that are preferably of high purity and are substantially free of potentially harmful contaminants, including proteins (e.g., at least National Food (NF) grade, generally at least analytical grade, and more typically at least pharmaceutical grade). Compositions are at least 80, at least 90, at least 99 or at least 99.9% w/w pure of undesired contaminants such as proteins are substantially free of protein. To the extent that a given compound must be synthesized prior to use, the resulting product is typically substantially free of any potentially toxic agents, particularly any endotoxins, which may be present during the synthesis or purification process. Compositions are usually made under GMP conditions. Compositions for parenteral administration are usually sterile and substantially isotonic.

### I General

Polysaccharides form a heterogeneous group of polymers of different length and composition. They are constructed from monosaccharide residues that are linked by glycosidic bonds. Glycosidic linkages may be located between the C₁ (or C₂) of one sugar residue and the C₂, C₃, C₄, C₅ or C₆ of the second residue. A branched sugar results if more than two types of linkage are present in single monosaccharide molecule.

Monosaccharides are simple sugars with multiple hydroxyl groups. Based on the number of carbons (e.g., 3, 4, 5, or 6) a monosaccharide is a triose, tetrose, pentose, or hexose. Pentoses and hexoses can cyclize, as the aldehyde or keto group reacts with a hydroxyl on one of the distal carbons. Examples of monosaccharides are galactose, glucose, and rhamnose.

Polysaccharides are molecules comprising a plurality of monosaccharides covalently linked to each other through glycosidic bonds. Polysaccharides consisting of a relatively small number of monosaccharide units, such as 10 or less, are sometimes referred to as oligosaccharides. The end of the polysaccharide with an anomeric carbon (C₁) that is not involved in a glycosidic bond is called the reducing end. A polysaccharide may consist of one monosaccharide type, known as a homopolymer, or two or more types of monosaccharides, known as a heteropolymer. Examples of homopolysaccharides are cellulose, amylose, inulin, chitin, chitosan, amylopectin, glycogen, and pectin. Amylose is a glucose polymer with α(1→4) glycosidic linkages. Amylopectin is a glucose polymer with α(1 →4) linkages and branches formed by α(1→6) linkages. Examples of heteropolysaccharides are glucomannan, galactoglucomannan, xyloglucan, 4-O-methylglucuronoxylan, arabinoxylan, and 4-O-Methylglucuronoarabinoxylan.

Polysaccharides can be structurally modified both enzymatically and chemically. Examples of modifications include sulfation, phosphorylation, methylation, O-acetylation, fatty acylation, amino N-acetylation, N-sulfation, branching, and carboxyl lactonization.

Glycosaminoglycans are polysaccharides of repeating disaccharides. Within the disaccharides, the sugars tend to be modified, with acidic groups, amino groups, sulfated hydroxyl and amino groups. Glycosaminoglycans tend to be negatively charged, because of the prevalence of acidic groups. Examples of glycosaminoglycans are heparin, chondroitin, and hyaluronic acid.

Polysaccharides are produced in eukaryotes mainly in the endoplasmic reticulum (ER) and Golgi apparatus. Polysaccharide biosynthesis enzymes are usually retained in the ER, and amino acid motifs imparting ER retention have been identified (Gene. 2000 Dec. 31; 261(2):321-7). Polysaccharides are also produced by some prokaryotes, such as lactic acid bacteria.

Polysaccharides that are secreted from cells are known as exopolysaccharides. Many types of cell walls, in plants, algae, and bacteria, are composed of polysaccharides. The cell walls are formed through secretion of polysaccharides. Some species, including algae and bacteria, secrete polysaccharides that are released from the cells. In other words, these molecules are not held in association with the cells as are cell wall polysaccharides. Instead, these molecules are released from the cells. For example, cultures of some species of microalgae secrete exopolysaccharides that are suspended in the culture media.

### II Methods of Producing Polysaccharides

A. Cell Culture Methods: Microalgae

Polysaccharides can be produced by culturing microalgae. Examples of microalgae that can be cultured to produce polysaccharides are shown in Table 1. Also listed are references that enable the skilled artisan to culture the microalgae species under conditions sufficient for polysaccharide production. Also listed are strain numbers from various publicly available algae collections, as well as strains published in journals that require public dissemination of reagents as a prerequisite for publication.

TABLE 1

| | | Culture and | | |
|---|---|---|---|---|
| | | polysaccharide | Reported | |
| | Strain Number/ | purification method | Monosaccharide | |
| Species | Source | reference | Composition | Culture conditions |
| Porphyridium | UTEX¹ 161 | M. A. Guzman-Murillo | Xylose, Glucose, | Cultures obtained from various sources and |
| cruentum | | and F. Ascencio., | Galactose, | were cultured in F/2 broth prepared with |
| | | Letters in Applied | Glucoronic acid | seawater filtered through a 0.45 um Millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Porphyridium | UTEX 161 | Fabregas et al., | Xylose, Glucose, | Cultured in 80 ml glass tubes with aeration of |
| cruentum | | Antiviral Research | Galactose and | 100 ml/min and 10% CO₂, for 10 s every ten |
| | | 44(1999)-67-73 | Glucoronic acid | minutes to maintain pH > 7.6. Maintained at 22° |
| | | | | in 12:12 Light/dark periodicity. Light at |
| | | | | 152.3 umol/m2/s. Salinity 3.5% (nutrient enriched as |
| | | | | Fabregas, 1984 modified in 4 mmol Nitrogen/L) |
| Porphyridium | UTEX 637 | Dvir, Brit. J. of | Xylose, Glucose | Outdoor cultivation for 21 days in artficial sea |
| sp. | | Nutrition (2000), 84, | and Galactose, | water in polyethylene sleeves. See Jones (1963) |
| | | 469-476. [Review: S. Geresh | Methyl hexoses, | and Cohen & Malis Arad, 1989) |
| | | Biosource | Mannose, | |
| | | Technology 38 (1991) | Rhamnose | |
| | | 195-201]-Huleihel, | | |
| | | 2003, Applied | | |
| | | Spectoscopy, v57, No. | | |
| | | 4 2003 | | |
| Porphyridium | SAG² 111.79 | Talyshinsky, Marina | xylose, glucose, | see Dubinsky et al. Plant Physio. And Biochem. |
| aerugineum | | Cancer Cell Int'l 2002, | galactose, methyl | (192) 30: 409-414. Pursuant to Ramus_1972--> |
| | | 2; Review: S. Geresh | hexoses | Axenic culutres are grown in MCYII liquid |
| | | Biosource Technology | | medium at 25° C. and illuminated with Cool |
| | | 38 (1991) 195-201]1 | | White fluorescent tubes on a 16:8 hr light dark |
| | | See Ramus_1972 | | cycle. Cells kept in suspension by agitation on a |
| | | | | gyrorotary shaker or by a stream of filtered air. |
| Porphyridium | strain 1380-1a | Schmitt D., Water | | See cited reference |
| purpurpeum | | Research | | |
| | | Volume 35, Issue 3, | | |
| | | March 2001, Pages | | |
| | | 779-785, Bioprocess | | |
| | | Biosyst Eng. 2002 | | |
| | | Apr; 25(1): 35-42. Epub | | |
| | | 2002 Mar 6 | | |
| Chaetoceros sp. | USCE³ | M. A. Guzman-Murillo | | See cited reference |
| | | and F. Ascencio., | | |
| | | Letters in Applied | | |
| | | Microbiology 2000, 30, | | |
| | | 473-478 | | |
| Chlorella | USCE | M. A. Guzman-Murillo | | See cited reference |
| autotropica | | and F. Ascencio., | | |
| | | Letters in Applied | | |
| | | Microbiology 2000, 30, | | |
| | | 473-478 | | |
| Chlorella | UTEX 580 | Fabregas et al., | | Cultured in 80 ml glass tubes with aeration of |
| autotropica | | Antiviral Research | | 100 ml/min and 10% CO2, for 10 s every ten |
| | | 44(1999)-67-73 | | minutes to maintain pH > 7.6. Maintained at 22° |
| | | | | in 12:12 Light/dark periodicity. Light at |
| | | | | 152.3 umol/m2/s. Salinity 3.5% (nutrient enriched as |
| | | | | Fabregas, 1984) |
| Chlorella | UTEX LB2074 | M. A. Guzman-Murillo | | Cultures obtained from various sources and |
| capsulata | | and F. Ascencio., | | were cultured in F/2 broth prepared with |
| | | Letters in Applied | | seawater filtered through a 0.45 um Millipore |
| | | Microbiology 2000, 30, | | filter or distilled water depending on microalgae |
| | | 473-478 (species is | | salt tolerance. Incubated at 25° C. in flasks and |
| | | a.k.a. | | illuminated with white fluorescent lamps. |
| | | Schizochlamydella | | |
| | | capsulata) | | |
| Chlorella | GGMCC⁴ | S. Guzman, | glucose, | Grown in 10 L of membrane filtered (0.24 um) |
| stigmatophora | | Phytotherapy Rscrh | glucuronic acid, | seawater and sterilized at 120° for 30 min and |
| | | (2003) 17: 665-670 | xylose, | enriched with Erd Schreiber medium. Cultures |
| | | | ribose/fucose | maintained at 18 +/− 1° C. under constant 1% |
| | | | | CO₂ bubbling. |
| Chlorella | UTEX 2219; | U.S. Pat. No. 4,831,020 | | See cited references |
| minutissima | UTEX 2341 | and 4,533,548 | | |
| Chlorella | UTEX 1806; | U.S. Pat. No. 6,974,576, | | See cited references |
| pyrenoidosa | UTEX UTEX | 6,551,596, and | | |
| | 343; UTEX 251; | 6,977,076 | | |
| | UTEX 252; | | | |
| | UTEX 260; | | | |
| | UTEX 1230; | | | |
| | UTEX 1662; | | | |
| | UTEX 1671; | | | |
| | UTEX 26; | | | |
| | UTEX 395 | | | |
| Dunalliela | DCCBC⁵ | Fabregas et al., | | Cultured in 80 ml glass tubes with aeration of |
| tertiolecta | | Antiviral Research | | 100 ml/min and 10% CO2, for 10 s every ten |
| | | 44(1999)-67-73 | | minutes to maintain pH > 7.6. Maintained at 22° |
| | | | | in 12:12 Light/dark periodicity. Light at |
| | | | | 152.3 umol/m2/s. Salinity 3.5% (nutrient enriched as |
| | | | | Fabregas, 1984) |
| Dunalliela | DCCBC | Fabregas et al., | | Cultured in 80 ml glass tubes with aeration of |
| bardawil | | Antiviral Research | | 100 ml/min and 10% CO2, for 10 s every ten |
| | | 44(1999)-67-73 | | minutes to maintain pH > 7.6. Maintained at 22° |
| | | | | in 12:12 Light/dark periodicity. Light at |
| | | | | 152.3 umol/m²/s. Salinity 3.5% (nutrient enriched as |
| | | | | Fabregas, 1984) |
| Isochrysis | HCTMS⁶ | M. A. Guzman-Murillo | | Cultures obtained from various sources and |
| galbana var. | | and F. Ascencio., | | were cultured in F/2 broth prepared with |
| tahitiana | | Letters in Applied | | seawater filtered through a 0.45 um millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Isochrysis | UTEX LB 987 | Fabregas et al., | | Cultured in 80 ml glass tubes with aeration of |
| galbana var. | | Antiviral Research | | 100 ml/min and 10% CO2, for 10 s every ten |
| Tiso | | 44(1999)-67-73 | | minutes to maintain pH > 7.6. Maintained at 22° |
| | | | | in 12:12 Light/dark periodicity. Light at |
| | | | | 152.3 umol/m²/s. Salinity 3.5% (nutrient enriched as |
| | | | | Fabregas, 1984) |
| Isochrysis sp. | CCMP⁷ | M. A. Guzman-Murillo | | Cultures obtained from various sources and |
| | | and F. Ascencio., | | were cultured in F/2 broth prepared with |
| | | Letters in Applied | | seawater filtered through a 0.45 um Millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Phaeodactylum | UTEX 642, 646, | M. A M. A. Guzman- | | Cultures obtained from various sources and |
| tricornutum | 2089 | Murillo and F. Ascencio., | | were cultured in F/2 broth prepared with |
| | | Letters in | | seawater filtered through a 0.45 um Millipore |
| | | Applied Microbiology | | filter or distilled water depending on microalgae |
| | | 2000, 30, 473-478 | | salt tolerance. Incubated at 25° C. in flasks and |
| | | | | illuminated with white fluorescent lamps. |
| Phaeodactylum | GGMCC | S. Guzman, | glucose, | Grown in 10 L of membrane filtered (0.24 um) |
| tricornutum | | Phytotherapy Rscrh | glucuronic acid, | seawater and sterilized at 120° for 30 min and |
| | | (2003) 17: 665-670 | and mannose | enriched with Erd Schreiber medium. Cultures |
| | | | | maintained at 18 +/− 1° C. under constant 1% |
| | | | | CO2 bubbling. |
| Tetraselmis sp. | CCMP 1634-1640; | M. A. Guzman-Murillo | | Cultures obtained from various sources and |
| | UTEX | and F. Ascencio., | | were cultured in F/2 broth prepared with |
| | 2767 | Letters in Applied | | seawater filtered through a 0.45 um Millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Botrycoccus | UTEX 572 and | M. A. Guzman-Murillo | | Cultures obtained from various sources and |
| braunii | 2441 | and F. Ascencio., | | were cultured in F/2 broth prepared with |
| | | Letters in Applied | | seawater filtered through a 0.45 um Millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Cholorococcum | UTEX 105 | M. A. Guzman-Murillo | | Cultures obtained from various sources and |
| | | and F. Ascencio., | | were cultured in F/2 broth prepared with |
| | | Letters in Applied | | seawater filtered through a 0.45 um Millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Hormotilopsis | UTEX 104 | M. A. Guzman-Murillo | | Cultures obtained from various sources and |
| gelatinosa | | and F. Ascencio., | | were cultured in F/2 broth prepared with |
| | | Letters in Applied | | seawater filtered through a 0.45 um Millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Neochloris | UTEX 1185 | M. A. Guzman-Murillo | | Cultures obtained from various sources and |
| oleoabundans | | and F. Ascencio., | | were cultured in F/2 broth prepared with |
| | | Letters in Applied | | seawater filtered through a 0.45 um Millipore |
| | | Microbiology 2000, 30, | | filter or distilled water depending on microalgae |
| | | 473-478 | | salt tolerance, incubated at 25° C. in flasks and |
| | | | | illuminated with white fluorescent lamps. |
| Ochromonas | UTEX L1298 | M. A. Guzman-Murillo | | Cultures obtained from various sources and |
| Danica | | and F. Ascencio., | | were cultured in F/2 broth prepared with |
| | | Letters in Applied | | seawater filtered through a 0.45 um Millipore |
| | | Microbiology 2000, 30, | | filter or distilled water depending on microalgae |
| | | 473-478 | | salt tolerance. Incubated at 25° C. in flasks and |
| | | | | illuminated with white fluorescent lamps. |
| Gyrodinium | KG03; KGO9; | Yim, Joung Han et. Al., | Homopolysaccharide | Isolated from seawater collected from red-tide |
| impudicum | KGJO1 | J. of Microbiol December | of galactose | bloom in Korean coastal water. Maintained in |
| | | 2004, 305-14; Yim, J. H. | w/ 2.96% uronic | f/2 medium at 22° under circadian light at |
| | | (2000) Ph.D. | acid | 100 uE/m2/sec: dark cycle of 14 h: 10 h for 19 |
| | | Dissertations, | | days. Selected with neomycin and/or |
| | | University of Kyung | | cephalosporin 20 ug/ml |
| | | Hee, Seoul | | |
| Ellipsoidon sp. | See cited | Fabregas et al., | | Cultured in 80 ml glass tubes with aeration of |
| | references | Antiviral Research | | 100 ml/min and 10% CO2, for 10 s every ten |
| | | 44(1999)-67-73; Lewin, R. A. | | minutes to maintain pH > 7.6. Maintained at 22° |
| | | Cheng, L., 1989. | | in 12:12 Light/dark periodicity. Light at |
| | | Phycologya 28, 96-108 | | 152.3 umol/m2/s. Salinity 3.5% (nutrient enriched as |
| | | | | Fabregas, 1984) |
| Rhodella | UTEX 2320 | Talyshinsky, Marina | | See Dubinsky O. et al. Composition of Cell wall |
| reticulata | | Cancer Cell Int'l 2002, 2 | | polysaccharide produced by unicellular red |
| | | | | algae Rhodella reticulata. 1992 Plant |
| | | | | Physiology and biochemistry 30: 409-414 |
| Rhodella | UTEX LB 2506 | Evans, LV., et al. J. | Galactose, xylose, | Grown in either SWM3 medium or ASP12, |
| maculata | | Cell Sci 16, 1-21 | glucuronic acid | MgCl2 supplement. 100 mls in 250 mls |
| | | (1974); EVANS, L. V. | | volumetric Erlenmeyer flask with gentle |
| | | (1970). Br. phycol. J. | | shaking and 4000lx Northern Light fluorescent |
| | | 5, 1-13. | | light for 16 hours. |
| Gymnodinium | Oku-1 | Sogawa, K., et al., Life | | See cited reference |
| sp. | | Sciences, Vol. 66, No. | | |
| | | 16, pp. PL 227-231 | | |
| | | (2000) AND | | |
| | | Umermura, Ken: | | |
| | | Biochemical | | |
| | | Pharmacology 66 | | |
| | | (2003) 481-487 | | |
| Spirilina | UTEX LB 1926 | Kaji, T et. Al., Life Sci | Na-Sp contains | See cited reference |
| platensis | | 2002 Mar | two disaccharide | |
| | | 8; 70(16): 1841-8 | repeats: | |
| | | Schaeffer and Krylov | Aldobiuronic acid | |
| | | (2000) Review- | and Acofriose + | |
| | | Ectoxicology and | other minor | |
| | | Environmental Safety. | saccharides and | |
| | | 45, 208-227. | sodium ion | |
| Cochlodinuium | Oku-2 | Hasui., et. Al., Int. J. | mannose, | Precultures grown in 500 ml conicals containing |
| polykrikoides | | Bio. Macromol. | galactose, glucose | 300 mls ESM (?) at 21.5° C. for 14 days in |
| | | Volume 17 No. 5 1995. | and uronic acid | continuous light (3500 lux) in growth cabinet) |
| | | | | and then transferred to 5 liter conical flask |
| | | | | containing 3 liters of ESM. Grown 50 days and |
| | | | | then filtered thru wortmann GFF filter. |
| Nostoc | PCC⁸ 7413, | Sangar, VK Applied | | Growth in nitrogen fixing conditions in BG-11 |
| muscorum | 7936, 8113 | Micro. (1972) & A. M. Burja | | medium in aerated cultures maintained in log |
| | | et al Tetrahydron | | phase for several months. 250 mL culture media |
| | | 57 (2001) 937-9377; | | that were disposed in a temperature controlled |
| | | Otero A., J Biotechnol. | | incubator and continuously illuminated with |
| | | 2003 Apr | | 70umol photon m−2 s−1 at 30° C.. |
| | | 24; 102(2): 143-52 | | |
| Cyanospira | See cited | A. M. Burja et al. | | See cited reference |
| capsulata | references | Tetrahydron 57 (2001) | | |
| | | 937-9377 & Garozzo, D., | | |
| | | Carbohydrate Res. | | |
| | | 1998 307 113-124; | | |
| | | Ascensio, F., Folia | | |
| | | Microbiol (Praha). | | |
| | | 2004; 49(1): 64-70., | | |
| | | Cesaro, A., et al., Int J | | |
| | | Biol Macromol. 1990 | | |
| | | Apr; 12(2): 79-84 | | |
| Cyanothece sp. | ATCC 51142 | Ascensio F., Folia | | Maintained at 27° C. in ASN III medium with |
| | | Microbiol (Praha). | | light/dark cycle of 16/8 h under fluorescent light |
| | | 2004; 49(1): 64-70. | | of 3,000 lux light intensity. In Phillips each of |
| | | | | 15 strains were grown photoautotrophically in |
| | | | | enriched seawater medium. When required the |
| | | | | amount of NaNO3 was reduced from 1.5 to 0.35 g/L. |
| | | | | Strains axenically grown in an atmosphere |
| | | | | of 95% air and 5% CO2 for 8 days under |
| | | | | continuous illmnination. with mean photon flux |
| | | | | of 30umol photon/m2/s for the first 3 days of |
| | | | | growth and 80 umol photon/m/s |
| Chlorella | UTEX 343; | Cheng_2004 Journal of | | See cited reference |
| pyrenoidosa | UTEX 1806 | Medicinal Food 7(2) | | |
| | | 146-152 | | |
| Phaeodactylum | CCAP 1052/1A | Fabregas et al., | | Cultured in 80 ml glass tubes with aeration of |
| tricornutum | | Antiviral Research | | 100 ml/min and 10% CO2, for 10 s every ten |
| | | 44(1999)-67-73 | | minutes to maintain pH > 7.6. Maintained at 22° |
| | | | | in 12:12 Light/dark periodicity. Light at |
| | | | | 152.3 umol/m2/s. Salinity 3.5% (nutrient enriched as |
| | | | | Fabregas, 1984) |
| Chlorella | USCE | M. A. Guzman-Murillo | | See cited reference |
| autotropica | | and F. Ascencio., | | |
| | | Letters in Applied | | |
| | | Microbiology 2000, 30, | | |
| | | 473-478 | | |
| Chlorella sp. | CCM | M. A. Guzman-Murillo | | See cited reference |
| | | and F. Ascencio., | | |
| | | Letters in Applied | | |
| | | Microbiology 2000, 30, | | |
| | | 473-478 | | |
| Dunalliela | USCE | M. A. Guzman-Murillo | | See cited reference |
| tertiolecta | | and F. Ascencio., | | |
| | | Letters in Applied | | |
| | | Microbiology 2000, 30, | | |
| | | 473-478 | | |
| Isochrysis | UTEX LB 987 | Fabregas et al., | | Cultured in 80 ml glass tubes with aeration of |
| galabana | | Antiviral Research | | 100 ml/min and 10% CO₂, for 10 s every ten |
| | | 44(1999)-67-73 | | minutes to maintain pH > 7.6. Maintained at 22° |
| | | | | in 12:12 Light/dark periodicity. Light at |
| | | | | 152.3 umol/m2/s. Salinity 3.5% (nutrient enriched as |
| | | | | Fabregas, 1984) |
| Tetraselmis | CCAP 66/1A-D | Fabregas et al., | | Cultured in 80 ml glass tubes with aeration of |
| tetrathele | | Antiviral Research | | 100 ml/min and 10% CO₂, for 10 s every ten |
| | | 44(1999)-67-73 | | minutes to maintain pH > 7.6. Maintained at 22° |
| | | | | in 12:12 Light/dark periodicity. Light at |
| | | | | 152.3 umol/m2/s. Salinity 3.5% (nutrient enriched as |
| | | | | Fabregas, 1984) |
| Tetraselmis | UTEX LB 2286 | M. A. Guzman-Murillo | | See cited reference |
| suecica | | and F. Ascencio., | | |
| | | Letters in Applied | | |
| | | Microbiology 2000, 30, | | |
| | | 473-478 | | |
| Tetraselmis | CCAP 66/4 | Fabregas et al., | | Cultured in 80 ml glass tubes with aeration of |
| suecica | | Antiviral Research | | 100 ml/min and 10% CO₂, for 10 s every ten |
| | | 44(1999)-67-73 and | | minutes to maintain pH > 7.6. Maintained at 22° |
| | | Otero and Fabregas- | | in 12:12 Light/dark periodicity. Light at |
| | | Aquaculture 159 (1997) | | 152.3 umol/m2/s. Salinity 3.5% (nutrient enriched as |
| | | 111-123. | | Fabregas, 1984) |
| Botrycoccus | UTEX 2629 | M. A. Guzman-Murillo | | See cited reference |
| sudeticus | | and F. Ascencio., | | |
| | | Letters in Applied | | |
| | | Microbiology 2000, 30, | | |
| | | 473-478 | | |
| Chlamydomonas | UTEX 729 | Moore and Tisher | | See cited reference |
| mexicana | | Science. 1964 Aug | | |
| | | 7; 145: 586-7. | | |
| Dysmorphococcus | UTEX LB 65 | M. A. Guzman-Murillo | | See cited reference |
| globosus | | and F. Ascencio., | | |
| | | Letters in Applied | | |
| | | Microbiology 2000, 30, | | |
| | | 473-478 | | |
| Rhodella | UTEX LB 2320 | S. Geresh et al., J | | See cited reference |
| reticulata | | Biochem. Biophys. | | |
| | | Methods 50 (2002) | | |
| | | 179-187 [Review: S. Geresh | | |
| | | Biosource | | |
| | | Technology 38 (1991) | | |
| | | 195-201] | | |
| Anabena | ATCC 29414 | Sangar, VK Appl | In Vegative wall | See cited reference |
| cylindrica | | Microbiol. 1972 | where only 18% is | |
| | | Nov; 24(5): 732-4 | carbohydrate-- | |
| | | | Glucose [35%], | |
| | | | mannose [50%], | |
| | | | galactose, xylose, | |
| | | | and fucose. In | |
| | | | heterocyst wall | |
| | | | where 73% is | |
| | | | carbohydrate-- | |
| | | | Glucose 73% and | |
| | | | Mannose is 21% | |
| | | | with some | |
| | | | galactose and | |
| | | | xylose | |
| Anabena flos- | A37; JM | Moore, BG [1965] Can | Glucose and | See cited reference and APPLIED |
| aquae | Kingsbury | J. Microbiol. | mannose | ENVIRONMENTAL MICROBIOLOGY, April |
| | Laboratory, | Dec; 11(6): 877-85 | | 1978, 718-723) |
| | Cornell | | | |
| | University | | | |
| Palmella | See cited | Sangar, VK Appl | | See cited reference |
| mucosa | references | Microbiol. 1972 | | |
| | | Nov; 24(5): 732-4; | | |
| | | Lewin RA., (1956) Can | | |
| | | J Microbiol. 2: 665-672; | | |
| | | Arch Mikrobiol. | | |
| | | 1964 Aug 17; 49: 158-66 | | |
| Anacystis | PCC 6301 | Sangar, VK Appl | Glucose, | See cited reference |
| nidulans | | Microbiol. 1972 | galactose, | |
| | | Nov; 24(5): 732-4 | mannose | |
| Phormidium | See cited | Vicente-Garcia V. et | Galactose, | Cultivated in 2 L BG-11 medium at 28° C.. |
| 94a | reference | al., Biotechnol Bioeng. | Mannose, | Acetone was added to precipitate |
| | | 2004 Feb 5; 85(3): 306-10 | Galacturonic acid, | exopolysaccharide. |
| | | | Arabinose, and | |
| | | | Ribose | |
| Anabaenaopsis | 1402/1⁹ | David KA, Fay P. | | See cited reference |
| circularis | | Appl Environ | | |
| | | Microbiol. 1977 | | |
| | | Dec; 34(6): 640-6 | | |
| Aphanocapsa | MN-11 | Sudo H., et al., Current | Rhamnose; | Cultured aerobically for 20 days in seawater- |
| halophtia | | Micrcobiology Vol. 30 | mannose; fucose; | based medium, with 8% NaCl, and 40 mg/L |
| | | (1995), pp.219-222 | galactose; xylose; | NaHPO4. Nitrate changed the |
| | | | glucose In ratio of: | Exopolysaccharide content. Highest cell density |
| | | | 15:53:3:3:25 | was obtained from culture supplemented with |
| | | | | 100 mg/l NaNO₃. Phosphorous (40 mg/L) |
| | | | | could be added to control the biomass and |
| | | | | exopolysaccharide concentration. |
| Aphanocapsa | See reference | De Philippis R et al., | | Incubated at 20 and 28° C. with artificial light at |
| sp | | Sci Total Environ. 2005 | | a photon flux of 5-20 umol m⁻² s⁻¹. |
| | | Nov 2; | | |
| Cylindrotheca | See reference | De Philippis R et al., | Glucuronic acid, | Stock enriched cultures incubated at 20 and |
| sp | | Sci Total Environ. 2005 | Galacturonic acid, | 28° C. with artificial light at a photon flux of |
| | | Nov 2; | Glucose, | 5-20 umol m−2 s−1. Exopolysaccharide production |
| | | | Mannose, | done in glass tubes containing 100 mL culture |
| | | | Arabinose, | at 28° C. with continuous illumination at photon |
| | | | Fructose and | density of 5-10 uE m−2 s−1. |
| | | | Rhamnose | |
| Navicula sp | See reference | De Philippis R et al., | Glucuronic acid, | Incubated at 20 and 28° C. with artificial light at |
| | | Sci Total Environ. 2005 | Galacturonic acid, | a photon flux of 5-20 umol m−2 s−1. EPS |
| | | Nov 2; | Glucose, | production done in glass tubes containing 100 mL |
| | | | Mannose, | culture at 28° C. with continuous |
| | | | Arabinose, | illumination at photon density of 5-10 uE m−2 s−1. |
| | | | Fructose and | |
| | | | Rhamnose | |
| Gloeocapsa sp | See reference | De Philippis R et al., | | Incubated at 20 and 28° C. with artifical light at a |
| | | Sci Total Environ. 2005 | | photon flux of 5-20 umol m−2 s−1. |
| | | Nov 2; | | |
| Gloeocapsa | See cited | J Appl Microbiol. | | See cited references |
| alpicola | references | 2005; 98(1): 114-20; | | |
| | | Photochem Photobiol. | | |
| | | 1982 Mar; 35(3): 359-64; | | |
| | | J Gen Microbiol. | | |
| | | 1977 Jan; 98(1): 277-80; | | |
| | | Arch Microbiol. 1976 | | |
| | | Feb; 107(1): 93-7; | | |
| | | FEMS Microbiol Lett. | | |
| | | 2002 Sep | | |
| | | 10; 214(2): 229-33 | | |
| Phaeocystis | See cited | Toxicology. 2004 Jul | | See cited references |
| pouchetii | references | 1; 199(2-3): 207-17; | | |
| | | Toxicon. 2003 | | |
| | | Jun; 41(7): 803-12; | | |
| | | Protist. 2002 | | |
| | | Sep; 153(3): 275-82; J | | |
| | | Virol. 2005 | | |
| | | Jul; 79(14): 9236-43; J | | |
| | | Bacteriol. 1961 | | |
| | | Jul; 82(1): 72-9 | | |
| Leptolyngbya | See reference | De Philippis R et al., | | Incubated at 20 and 28° C. with artificial light at |
| sp | | Sci Total Environ. 2005 | | a photon flux of 5-20 umol m−2 s−1. |
| | | Nov 2; | | |
| Symploca sp. | See reference | De Philippis R et al., | | Incubated at 20 and 28° C. with artificial light at |
| | | Sci Total Environ. 2005 | | a photon flux of 5-20 umol m−2 s−1. |
| | | Nov 2; | | |
| Synechocystis | PCC 6714/6803 | Jurgens UJ, Weckesser J. | Glucoseamine, | Photoautotrophically grown in BG-11 medium, |
| | | J Bacteriol. 1986 | mannosamine | pH 7.5 at 25° C.. Mass cultures prepared in a 12 |
| | | Nov; 168(2): 568-73 | galactosamine, | liter fermentor and gassed by air and carbon |
| | | | mannose and | dioxide at flow rates of 250 and 2.5 liters/h, |
| | | | glucose | with illumination from white fluorescent lamps |
| | | | | at a constant light intensity of 5,000 lux. |
| Stauroneis | See reference | Lind, JL (1997) Planta | | See cited reference |
| decipiens | | 203: 213-221 | | |
| Achnanthes | Indiana | Holdsworth, RH., Cell | | See cited reference |
| brevipes | University | Biol. 1968 | | |
| | Culture | Jun; 37(3): 831-7; Acta | | |
| | Collection | Cient Venez. | | |
| | | 2002; 53(1): 7-14.; J. | | |
| | | Phycol 36 pp. 882-890 | | |
| | | (2000) | | |
| Achnanthes | Strain 330 from | Wang, Y., et al., Plant | | See cited reference |
| longipes | National Institute | Physiol. 1997 | | |
| | for | Apr; 113(4): 1071-1080. | | |
| | Environmental | | | |
| | Studies | | | |

Microalgae are preferably cultured in liquid media for polysaccharide production. Culture condition parameters can be manipulated to optimize total polysaccharide production as well as to alter the structure of polysaccharides produced by microalgae.

Microalgal culture media usually contains components such as a fixed nitrogen source, trace elements, a buffer for pH maintenance, and phosphate. Other components can include a fixed carbon source such as acetate or glucose, and salts such as sodium chloride, particularly for seawater microalgae. Examples of trace elements include zinc, boron, cobalt, copper, manganese, and molybdenum in, for example, the respective forms of ZnCl₂, H₃BO₃, CoCl₂.6H₂O, CuCl₂.2H₂O, MnCl₂.4H₂O and (NH₄)₆Mo₇O₂₄.4H₂O.

Some microalgae species can grow by utilizing a fixed carbon source such as glucose or acetate. Such microalgae can be cultured in bioreactors that do not allow light to enter. Alternatively, such microalgae can also be cultured in photobioreactors that contain the fixed carbon source and allow light to strike the cells. Such growth is known as heterotrophic growth. Any strain of microalgae, including those listed in Table 1, can be cultured in the presence of any one or more fixed carbon source including those listed in Tables 2 and 3.

TABLE 2

2,3-Butanediol
2-Aminoethanol
2′-Deoxy Adenosine
3-Methyl Glucose
Acetic Acid
Adenosine
Adenosine-5′-Monophosphate
Adonitol
Amygdalin
Arbutin
Bromosuccinic Acid
Cis-Aconitic Acid
Citric Acid
D,L-Carnitine
D,L-Lactic Acid
D,L-α-Glycerol Phosphate
D-Alanine
D-Arabitol
D-Cellobiose
Dextrin
D-Fructose
D-Fructose-6-Phosphate
D-Galactonic Acid Lactone
D-Galactose
D-Galacturonic Acid
D-Gluconic Acid
D-Glucosaminic Acid
D-Glucose-6-Phosphate
D-Glucuronic Acid
D-Lactic Acid Methyl Ester
D-L-α-Glycerol Phosphate
D-Malic Acid
D-Mannitol
D-Mannose
D-Melezitose
D-Melibiose
D-Psicose
D-Raffinose
D-Ribose
D-Saccharic Acid
D-Serine
D-Sorbitol
D-Tagatose
D-Trehalose
D-Xylose
Formic Acid
Gentiobiose
Glucuronamide
Glycerol
Glycogen
Glycyl-LAspartic Acid
Glycyl-LGlutamic Acid
Hydroxy-LProline
i-Erythritol
Inosine
Inulin
Itaconic Acid
Lactamide
Lactulose
L-Alaninamide
L-Alanine
L-Alanylglycine
L-Alanyl-Glycine
L-Arabinose
L-Asparagine
L-Aspartic Acid
L-Fucose
L-Glutamic Acid
L-Histidine
L-Lactic Acid
L-Leucine
L-Malic Acid
L-Ornithine
LPhenylalanine
L-Proline
L-Pyroglutamic Acid
L-Rhamnose
L-Serine
L-Threonine
Malonic Acid
Maltose
Maltotriose
Mannan
m-Inositol
N-Acetyl-DGalactosamine
N-Acetyl-DGlucosamine
N-Acetyl-LGlutamic Acid
N-Acetyl-β-DMannosamine
Palatinose
Phenyethylamine
p-Hydroxy-Phenylacetic Acid
Propionic Acid
Putrescine
Pyruvic Acid
Pyruvic Acid Methyl Ester
Quinic Acid
Salicin
Sebacic Acid
Sedoheptulosan
Stachyose
Succinamic Acid
Succinic Acid
Succinic Acid Mono-Methyl-Ester
Sucrose
Thymidine
Thymidine-5′-Monophosphate
Turanose
Tween 40
Tween 80
Uridine
Uridine-5′-Monophosphate
Urocanic Acid
Water
Xylitol
α-Cyclodextrin
α-D-Glucose
α-D-Glucose-1-Phosphate
α-D-Lactose
α-Hydroxybutyric Acid
α-Keto Butyric Acid
α-Keto Glutaric Acid
α-Keto Valeric Acid
α-Ketoglutaric Acid
α-Ketovaleric Acid
α-Methyl-DGalactoside
α-Methyl-DGlucoside
α-Methyl-DMannoside
β-Cyclodextrin
β-Hydroxybutyric Acid
β-Methyl-DGalactoside
β-Methyl-D-Glucoside
γ-Amino Butyric Acid
γ-Hydroxybutyric Acid

TABLE 3
(2-amino-3,4-dihydroxy-5-hydroxymethyl-1-cyclohexyl)glucopyranoside
(3,4-disinapoyl)fructofuranosyl-(6-sinapoyl)glucopyranoside
(3-sinapoyl)fructofuranosyl-(6-sinapoyl)glucopyranoside
1 reference
1,10-di-O-(2-acetamido-2-deoxyglucopyranosyl)-2-azi-1,10-decanediol
1,3-mannosylmannose
1,6-anhydrolactose
1,6-anhydrolactose hexaacetate
1,6-dichlorosucrose
1-chlorosucrose
1-desoxy-1-glycinomaltose
1-O-alpha-2-acetamido-2-deoxygalactopyranosyl-inositol
1-O-methyl-di-N-trifluoroacetyl-beta-chitobioside
1-propyl-4-O-beta galactopyranosyl-alpha galactopyranoside
2-(acetylamino)-4-O-(2-(acetylamino)-2-deoxy-4-O-sulfogalactopyranosyl)-2-deoxyglucose
2-(trimethylsilyl)ethyl lactoside
2,1′,3′,4′,6′-penta-O-acetylsucrose
2,2′-O-(2,2′-diacetamido-2,3,2′,3′-tetradeoxy-6,6′-di-O-(2-tetradecylhexadecanoyl)-
alpha,alpha′-trehalose-3,3′-diyl)bis(N-lactoyl-alanyl-isoglutamine)
2,3,6,2′,3′,4′,6′-hepta-O-acetylcellobiose
2,3′-anhydrosucrose
2,3-di-O-phytanyl-1-O-(mannopyranosyl-(2-sulfate)-(1-2)-glucopyranosyl)-sn-glycerol
2,3-epoxypropyl O-galactopyranosyl(1-6)galactopyranoside
2,3-isoprolylideneerthrofuranosyl 2,3-O-isopropylideneerythrofuranoside
2′,4′-dinitrophenyl 2-deoxy-2-fluoro-beta-xylobioside
2,5-anhydromannitol iduronate
2,6-sialyllactose
2-acetamido-2,4-dideoxy-4-fluoro-3-O-galactopyranosylglucopyranose
2-acetamido-2-deoxy-3-O-(gluco-4-enepyranosyluronic acid)glucose
2-acetamido-2-deoxy-3-O-rhamnopyranosylglucose
2-acetamido-2-deoxy-6-O-beta galactopyranosylgalactopyranose
2-acetamido-2-deoxyglucosylgalactitol
2-acetamido-3-O-(3-acetamido-3,6-dideoxy-beta-glucopyranosyl)-2-deoxy-galactopyranose
2-amino-6-O-(2-amino-2-deoxy-glucopyranosyl)-2-deoxyglucose
2-azido-2-deoxymannopyranosyl-(1,4)-rhamnopyranose
2-deoxy-6-O-(2,3-dideoxy-4,6-O-isopropylidene-2,3-(N-tosylepimino)mannopyranosyl)-
4,5-O-isopropylidene-1,3-di-N-tosylstreptamine
2-deoxymaltose
2-iodobenzyl-1-thiocellobioside
2-N-(4-benzoyl)benzoyl-1,3-bis(mannos-4-yloxy)-2-propylamine
2-nitrophenyl-2-acetamido-2-deoxy-6-O-beta galactopyranosyl-alpha galactopyranoside
2-O-(glucopyranosyluronic acid)xylose
2-O-glucopyranosylribitol-1-phosphate
2-O-glucopyranosylribitol-4′-phosphate
2-O-rhamnopyranosyl-rhamnopyranosyl-3-hydroxyldecanoyl-3-hydroxydecanoate
2-O-talopyranosylmannopyranoside
2-thiokojibiose
2-thiosophorose
3,3′-neotrehalosadiamine
3,6,3′,6′-dianhydro(galactopyranosylgalactopyranoside)
3,6-di-O-methyl-beta-glucopyranosyl-(1-4)-2,3-di-O-methyl-alpha-rhamnopyranose
3-amino-3-deoxyaltropyranosyl-3-amino-3-deoxyaltropyranoside
3-deoxy-3-fluorosucrose
3-deoxy-5-O-rhamnopyranosyl-2-octulopyranosonate
3-deoxyoctulosonic acid-(alpha-2-4)-3-deoxyoctulosonic acid
3-deoxysucrose
3-ketolactose
3-ketosucrose
3-ketotrehalose
3-methyllactose
3-O-(2-acetamido-6-O-(N-acetylneuraminyl)-2-deoxygalactosyl)serine
3-O-(glucopyranosyluronic acid)galactopyranose
3-O-beta-glucuronosylgalactose
3-O-fucopyranosyl-2-acetamido-2-deoxyglucopyranose
3′-O-galactopyranosyl-1-4-O-galactopyranosylcytarabine
3-O-galactosylarabinose
3-O-talopyranosylmannopyranoside
3-trehalosamine
4-(trifluoroacetamido)phenyl-2-acetamido-2-deoxy-4-O-beta-mannopyranosyl-beta-
glucopyranoside
4,4′6,6′-tetrachloro-4,4′6,6′-tetradeoxygalactotrehalose
4,6,4′,6′-dianhydro(galactopyranosylgalactopyranoside)
4,6-dideoxysucrose
4,6-O-(1-ethoxy-2-propenylidene)sucrose hexaacetate
4-chloro-4-deoxy-alpha-galactopyranosyl 3,4-anhydro-1,6-dichloro-1,6-dideoxy-beta-lyxo-
hexulofuranoside
4-glucopyranosylmannose
4-methylumbelliferylcellobioside
4-nitrophenyl 2-fucopyranosyl-fucopyranoside
4-nitrophenyl 2-O-alpha-D-galactopyranosyl-alpha-D-mannopyranoside
4-nitrophenyl 2-O-alpha-D-glucopyranosyl-alpha-D-mannopyranoside
4-nitrophenyl 2-O-alpha-D-mannopyranosyl-alpha-D-mannopyranoside
4-nitrophenyl 6-O-alpha-D-mannopyranosyl-alpha-D-mannopyranoside
4-nitrophenyl-2-acetamido-2-deoxy-6-O-beta-D-galactopyranosyl-beta-D-glucopyranoside
4-O-(2-acetamido-2-deoxy-beta-glucopyranosyl)ribitol
4-O-(2-amino-2-deoxy-alpha-glucopyranosyl)-3-deoxy-manno-2-octulosonic acid
4-O-(glucopyranosyluronic acid)xylose
4-O-acetyl-alpha-N-acetylneuraminyl-(2-3)-lactose
4-O-alpha-D-galactopyranosyl-D-galactose
4-O-galactopyranosyl-3,6-anhydrogalactose dimethylacetal
4-O-galactopyranosylxylose
4-O-mannopyranosyl-2-acetamido-2-deoxyglucose
4-thioxylobiose
4-trehalosamine
4-trifluoroacetamidophenyl 2-acetamido-4-O-(2-acetamido-2-deoxyglucopyranosyl)-2-
deoxymannopyranosiduronic acid
5-bromoindoxyl-beta-cellobioside
5′-O-(fructofuranosyl-2-1-fructofuranosyl)pyridoxine
5-O-beta-galactofuranosyl-galactofuranose
6 beta-galactinol
6(2)-thiopanose
6,6′-di-O-corynomycoloyl-alpha-mannopyranosyl-alpha-mannopyranoside
6,6-di-O-maltosyl-beta-cyclodextrin
6,6′-di-O-mycoloyl-alpha-mannopyranosyl-alpha-mannopyranoside
6-chloro-6-deoxysucrose
6-deoxy-6-fluorosucrose
6-deoxy-alpha-gluco-pyranosiduronic acid
6-deoxy-gluco-heptopyranosyl 6-deoxy-gluco-heptopyranoside
6-deoxysucrose
6-O-decanoyl-3,4-di-O-isobutyrylsucrose
6-O-galactopyranosyl-2-acetamido-2-deoxygalactose
6-O-galactopyranosylgalactose
6-O-heptopyranosylglucopyranose
6-thiosucrose
7-O-(2-amino-2-deoxyglucopyranosyl)heptose
8-methoxycarbonyloctyl-3-O-glucopyranosyl-mannopyranoside
8-O-(4-amino-4-deoxyarabinopyranosyl)-3-deoxyoctulosonic acid
allolactose
allosucrose
allyl 6-O-(3-deoxyoct-2-ulopyranosylonic acid)-(1-6)-2-deoxy-2-(3-
hydroxytetradecanamido)glucopyranoside 4-phosphate
alpha-(2-9)-disialic acid
alpha,alpha-trehalose 6,6′-diphosphate
alpha-glucopyranosyl alpha-xylopyranoside
alpha-maltosyl fluoride
aprosulate
benzyl 2-acetamido-2-deoxy-3-O-(2-O-methyl-beta-galactosyl)-beta-glucopyranoside
benzyl 2-acetamido-2-deoxy-3-O-beta fucopyranosyl-alpha-galactopyranoside
benzyl 2-acetamido-6-O-(2-acetamido-2,4-dideoxy-4-fluoroglucopyranosyl)-2-
deoxygalactopyranoside
benzyl gentiobioside
beta-D-galactosyl(1-3)-4-nitrophenyl-N-acetyl-alpha-D-galactosamine
beta-methylmelibiose
calcium sucrose phosphate
camiglibose
cellobial
cellobionic acid
cellobionolactone
Cellobiose
cellobiose octaacetate
cellobiosyl bromide heptaacetate
Celsior
chitobiose
chondrosine
Cristolax
deuterated methyl beta-mannobioside
dextrin maltose
D-glucopyranose, O-D-glucopyranosyl
Dietary Sucrose
difructose anhydride I
difructose anhydride III
difructose anhydride IV
digalacturonic acid
DT 5461
ediol
epilactose
epsilon-N-1-(1-deoxylactulosyl)lysine
feruloyl arabinobiose
floridoside
fructofuranosyl-(2-6)-glucopyranoside
FZ 560
galactosyl-(1-3)galactose
garamine
gentiobiose
geranyl 6-O-alpha-L-arabinopyranosyl-beta-D-glucopyranoside
geranyl 6-O-xylopyranosyl-glucopyranoside
glucosaminyl-1,6-inositol-1,2-cyclic monophosphate
glucosyl (1-4) N-acetylglucosamine
glucuronosyl(1-4)-rhamnose
heptosyl-2-keto-3-deoxyoctonate
inulobiose
Isomaltose
isomaltulose
isoprimeverose
kojibiose
lactobionic acid
lacto-N-biose II
Lactose
lactosylurea
Lactulose
laminaribiose
lepidimoide
leucrose
levanbiose
lucidin 3-O-beta-primveroside
LW 10121
LW 10125
LW 10244
maltal
maltitol
Maltose
maltose hexastearate
maltose-maleimide
maltosylnitromethane heptaacetate
maltosyltriethoxycholesterol
maltotetraose
Malun 25
mannosucrose
mannosyl-(1-4)-N-acetylglucosaminyl-(1-N)-urea
mannosyl(2)-N-acetyl(2)-glucose
melibionic acid
Melibiose
melibiouronic acid
methyl 2-acetamido-2-deoxy-3-O-(alpha-idopyranosyluronic acid)-4-O-sulfo-beta-
galactopyranoside
methyl 2-acetamido-2-deoxy-3-O-(beta-glucopyranosyluronic acid)-4-O-sulfo-beta-
galactopyranoside
methyl 2-acetamido-2-deoxy-3-O-glucopyranosyluronoylglucopyranoside
methyl 2-O-alpha-rhamnopyranosyl-beta-galactopyranoside
methyl 2-O-beta-rhamnopyranosyl-beta-galactopyranoside
methyl 2-O-fucopyranosylfucopyranoside 3 sulfate
methyl 2-O-mannopyranosylmannopyranoside
methyl 2-O-mannopyranosyl-rhamnopyranoside
methyl 3-O-(2-acetamido-2-deoxy-6-thioglucopyranosyl)galactopyranoside
methyl 3-O-galactopyranosylmannopyranoside
methyl 3-O-mannopyranosylmannopyranoside
methyl 3-O-mannopyranosyltalopyranoside
methyl 3-O-talopyranosyltalopyranoside
methyl 4-O-(6-deoxy-manno-heptopyranosyl)galactopyranoside
methyl 6-O-acetyl-3-O-benzoyl-4-O-(2,3,4,6-tetra-O-benzoylgalactopyranosyl)-2-deoxy-2-
phthalimidoglucopyranoside
methyl 6-O-mannopyranosylmannopyranoside
methyl beta-xylobioside
methyl fucopyranosyl(1-4)-2-acetamido-2-deoxyglucopyranoside
methyl laminarabioside
methyl O-(3-deoxy-3-fluorogalactopyranosyl)(1-6)galactopyranoside
methyl-2-acetamido-2-deoxyglucopyranosyl-1-4-glucopyranosiduronic acid
methyl-2-O-fucopyranosylfucopyranoside 4-sulfate
MK 458
N(1)-2-carboxy-4,6-dinitrophenyl-N(6)-lactobionoyl-1,6-hexanediamine
N-(2,4-dinitro-5-fluorophenyl)-1,2-bis(mannos-4′-yloxy)propyl-2-amine
N-(2′-mercaptoethyl)lactamine
N-(2-nitro-4-azophenyl)-1,3-bis(mannos-4′-yloxy)propyl-2-amine
N-(4-azidosalicylamide)-1,2-bis(mannos-4′-yloxy)propyl-2-amine
N,N-diacetylchitobiose
N-acetylchondrosine
N-acetyldermosine
N-acetylgalactosaminyl-(1-4)-galactose
N-acetylgalactosaminyl-(1-4)-glucose
N-acetylgalactosaminyl-1-4-N-acetylglucosamine
N-acetylgalactosaminyl-1-4-N-acetylglucosamine
N-acetylgalactosaminyl-alpha(1-3)galactose
N-acetylglucosamine-N-acetylmuramyl-alanyl-isoglutaminyl-alanyl-glycerol dipalmitoyl
N-acetylglucosaminyl beta(1-6)N-acetylgalactosamine
N-acetylglucosaminyl-1-2-mannopyranose
N-acetylhyalobiuronic acid
N-acetylneuraminoyllactose
N-acetylneuraminoyllactose sulfate ester
N-acetylneuraminyl-(2-3)-galactose
N-acetylneuraminyl-(2-6)-galactose
N-benzyl-4-O-(beta-galactopyranosyl)glucamine-N-carbodithioate
neoagarobiose
N-formylkansosaminyl-(1-3)-2-O-methylrhamnopyranose
O-((Nalpha)-acetylglucosamine 6-sulfate)-(1-3)-idonic acid
O-(4-O-feruloyl-alpha-xylopyranosyl)-(1-6)-glucopyranose
O-(alpha-idopyranosyluronic acid)-(1-3)-2,5-anhydroalditol-4-sulfate
O-(glucuronic acid 2-sulfate)-(1--3)-O-(2,5)-andydrotalitol 6-sulfate
O-(glucuronic acid 2-sulfate)-(1--4)-O-(2,5)-anhydromannitol 6-sulfate
O-alpha-glucopyranosyluronate-(1-2)-galactose
O-beta-galactopyranosyl-(1-4)-O-beta-xylopyranosyl-(1-0)-serine
octyl maltopyranoside
O-demethylpaulomycin A
O-demethylpaulomycin B
O-methyl-di-N-acetyl beta-chitobioside
Palatinit
paldimycin
paulomenol A
paulomenol B
paulomycin A
paulomycin A2
paulomycin B
paulomycin C
paulomycin D
paulomycin E
paulomycin F
phenyl 2-acetamido-2-deoxy-3-O-beta-D-galactopyranosyl-alpha-D-galactopyranoside
phenyl O-(2,3,4,6-tetra-O-acetylgalactopyranosyl)-(1-3)-4,6-di-O-acetyl-2-deoxy-2-
phthalimido-1-thioglucopyranoside
poly-N-4-vinylbenzyllactonamide
pseudo-cellobiose
pseudo-maltose
rhamnopyranosyl-(1-2)-rhamnopyranoside-(1-methyl ether)
rhoifolin
ruberythric acid
S-3105
senfolomycin A
senfolomycin B
solabiose
SS 554
streptobiosamine
Sucralfate
Sucrose
sucrose acetate isobutyrate
sucrose caproate
sucrose distearate
sucrose monolaurate
sucrose monopalmitate
sucrose monostearate
sucrose myristate
sucrose octaacetate
sucrose octabenzoic acid
sucrose octaisobutyrate
sucrose octasulfate
sucrose polyester
sucrose sulfate
swertiamacroside
T-1266
tangshenoside I
tetrahydro-2-((tetrahydro-2-furanyl)oxy)-2H-pyran
thionigerose
Trehalose
trehalose 2-sulfate
trehalose 6,6′-dipalmitate
trehalose-6-phosphate
trehalulose
trehazolin
trichlorosucrose
tunicamine
turanose
U 77802
U 77803
xylobiose
xylose-glucose
xylosucrose

Microalgae contain photosynthetic machinery capable of metabolizing photons, and transferring energy harvested from photons into fixed chemical energy sources such as monosaccharide. Glucose is a common monosaccharide produced by microalgae by metabolizing light energy and fixing carbon from carbon dioxide. Some microalgae can also grow in the absence of light on a fixed carbon source that is exogenously provided (for example see Plant Physiol. 2005 February; 137(2):460-74). In addition to being a source of chemical energy, monosaccharides such as glucose are also substrate for production of polysaccharides (see Example 14). The invention provides methods of producing polysaccharides with novel monosaccharide compositions. For example, microalgae is cultured in the presence of culture media that contains exogenously provided monosaccharide, such as glucose. The monosaccharide is taken up by the cell by either active or passive transport and incorporated into polysaccharide molecules produced by the cell. This novel method of polysaccharide composition manipulation can be performed with, for example, any microalgae listed in Table 1 using any monosaccharide or disaccharide listed in Tables 2 or 3.

In some embodiments, the fixed carbon source is one or more selected from glucose, galactose, xylose, mannose, rhamnose, N-acetylglucosamine, glycerol, floridoside, and glucuronic acid. The methods may be practiced cell growth in the presence of at least about 5.0 μM, at least about 10 μM, at least about 15.0 μM, at least about 20.0 μM, at least about 25.0 μM, at least about 30.0 μM, at least about 35.0 μM, at least about 40.0 μM, at least about 45.0 μM, at least about 50.0 μM, at least about 55.0 μM, at least about 60.0 μM, at least about 75.0 μM, at least about 80.0 μM, at least about 85.0 μM, at least about 90.0 μM, at least about 95.0 μM, at least about 100.0 μM, or at least about 150.0 μM, of one or more exogenously provided fixed carbon sources selected from Tables 2 and 3.

In some embodiments using cells of the genus Porphyridium, the methods include the use of approximately 0.5-0.75% glycerol as a fixed carbon source when the cells are cultured in the presence of light. Alternatively, a range of glycerol, from approximately 4.0% to approximately 9.0% may be used when the Porphyridium cells are cultured in the dark, more preferably from 5.0% to 8.0%, and more preferably 7.0%.

After culturing the microalgae in the presence of the exogenously provided carbon source, the monosaccharide composition of the polysaccharide can be analyzed as described in Example 5. Microalgae can be transformed with genes encoding carbohydrate transporters to facilitate the uptake of exogenously provided carbohydrates such SEQ ID NOs: 20, 22, 24, 26, 27, 29-39 and 46-48.

Microalgae culture media can contain a fixed nitrogen source such as KNO₃. Alternatively, microalgae are placed in culture conditions that do not include a fixed nitrogen source. For example, Porphyridium sp. cells are cultured for a first period of time in the presence of a fixed nitrogen source, and then the cells are cultured in the absence of a fixed nitrogen source (see for example Adda M., Biomass 10:131-140. (1986); Sudo H., et al., Current Microbiology Vol. 30 (1995), pp. 219-222; Marinho-Soriano E., Bioresour Technol. 2005 February; 96(3):379-82; Bioresour. Technol. 42:141-147 (1992)).

Other culture parameters can also be manipulated, such as the pH of the culture media, the identity and concentration of trace elements such as those listed in Table 3, and other media constituents. One non-limiting example is the inclusion of at least one source of sulfate in the culture media to increase the level of sulfation in the polysaccharides produced. In some embodiments, a polysaccharide preparation method is practiced with culture media containing more than about 100, more than about 150, more than about 200, more than about 250, more than about 300, more than about 350, more than about 400, more than about 450, more than about 500, more than about 550, more than about 600, more than about 650, more than about 700, or more than about 750, more than about 800, more than about 850, more than about 900, more than about 950, or more than about 1 or 2 M sulfate (or total SO₄²⁻). Increasing the sulfation has been demonstrated to increase the antioxidant apacity of the polysaccharide (see example 23 and Spitz et al. (J. Appl. Phycology (2005) 17:215-222). Without being bound by theory, and offered to improved the understanding of certain aspects of the disclosed invention, it is possible that an increased level of sulfation may increase the anti-cholesterol characteristics of the homogenized cell material or polysaccharide preparation disclosed herein. The correlation between higher amounts of sulfation and antioxidant activity demonstrated herein was unexpected given the weak antioxidant activity of carrageenan, which contains as much as 40% sulfate.

It is believed that microalgae of the genus Porphyridium have not been grown or propagated under conditions with sulfate concentrations of 100 mM to 2 M. Thus the invention includes the surprising discovery that microalgae are capable of growth under such conditions. Additionally, the invention is based in part on the surprising discovery that growth under higher sulfate concentrations can produce polysaccharides with higher levels of sulfation. This allows for the production of cells (and so biomass) containing highly sulfated polysaccharides that may be used in the form of purified polysaccharides, a homogenate of cells (biomass), intact cells (biomass) per se, or a combination thereof.

The discovery that Porphyridium can survive above 100 mM sulfate was surprising for a number of reasons. First, it is known that sulfate can alter the rate of uptake of toxic metal ions in algae, such as chromium, and that increasing metal accumulation can lead to toxicity (see for example Kaszycki et al, Plant, Cell & Environment, 28(2): p. 260, 2005). Second, it is also known that sulfate can inhibit the uptake of metal ions required for nitrogen fixation such as molybdenum, and that increasing sulfate concentrations negativelky affects algae such as cyanobacteria even at sulfate concentrations in estuarine (>8-10 mM) and seawater (28 mM) levels of sulfate (see for example Marino et al, Hydrobiologia 500: pp. 277-293, 2004). Third, sulfate at high levels can often be taken up and reduced and sulfide, which is toxic to photosynthesis because it attacks photosystem II (see for example Khanal et al., J. Envir. Engrg., 129(12); pp. 1104-1111, 2003). Fourth, high sulfate levels alter the osmotic pressure of the growth media, and many organisms cannot survive at such an elevated osmoticum. For example, it is well established that photosynthesis of algae is inhibited by hyperosmotic and salt stresses. See for example “Suppression of Quantum Yield of Photosystem II by Hyperosmotic Stress in Chlamydomonas reinhardtii” Plant Cell Physiol. 36: pp. 1253-1258 (1995); and “The Effect of Osmotic and Ionic Stress on the Primary Processes of Photosynthesis in Dunaliella tertiolecta”, J. Exp. Bot. 1984 35(1): 18-27 (1984).

Microalgae can be grown in the presence of light. The number of photons striking a culture of microalgae cells can be manipulated, as well as other parameters such as the wavelength spectrum and ratio of dark:light hours per day. Microalgae can also be cultured in natural light, as well as simultaneous and/or alternating combinations of natural light and artificial light. For example, microalgae of the genus Chlorella be cultured under natural light during daylight hours and under artificial light during night hours.

The gas content of a photobioreactor can be manipulated. Part of the volume of a photobioreactor can contain gas rather than liquid. Gas inlets can be used to pump gases into the photobioreactor. Any gas can be pumped into a photobioreactor, including air, air/CO₂ mixtures, noble gases such as argon and others. The rate of entry of gas into a photobioreactor can also be manipulated. Increasing gas flow into a photobioreactor increases the turbidity of a culture of microalgae. Placement of ports conveying gases into a photobioreactor can also affect the turbidity of a culture at a given gas flow rate. Air/CO₂ mixtures can be modulated to generate different polysaccharide compositions by manipulating carbon flux. For example, air:CO₂ mixtures of about 99.75% air:0.25% CO₂, about 99.5% air:0.5% CO₂, about 99.0% air:1.00% CO₂, about 98.0% air:2.0% CO₂, about 97.0% air:3.0% CO₂, about 96.0% air:4.0% CO₂, and about 95.00% air:5.0% CO₂ can be infused into a bioreactor or photobioreactor.

Microalgae cultures can also be subjected to mixing using devices such as spinning blades and propellers, rocking of a culture, stir bars, and other instruments.

B. Cell Culture Methods: Photobioreactors

Microalgae can be grown and maintained in closed photobioreactors made of different types of transparent or semitransparent material. Such material can include Plexiglas® enclosures, glass enclosures, bags bade from substances such as polyethylene, transparent or semitransparent pipes, and other materials. Microalgae can also be grown in open ponds.

Photobioreactors can have ports allowing entry of gases, solids, semisolids and liquids into the chamber containing the microalgae. Ports are usually attached to tubing or other means of conveying substances. Gas ports, for example, convey gases into the culture. Pumping gases into a photobioreactor can serve to both feed cells CO₂ and other gases and to aerate the culture and therefore generate turbidity. The amount of turbidity of a culture varies as the number and position of gas ports is altered. For example, gas ports can be placed along the bottom of a cylindrical polyethylene bag. Microalgae grow faster when CO₂ is added to air and bubbled into a photobioreactor. For example, a 5% CO₂:95% air mixture is infused into a photobioreactor containing cells of the genus Porphyridium (see for example Biotechnol Bioeng. 1998 Sep. 20; 59(6):705-13; textbook from office; U.S. Pat. Nos. 5,643,585 and 5,534,417; Lebeau, T., et. al. Appl. Microbiol. Biotechnol (2003) 60:612-623; Muller-Fuega, A., Journal of Biotechnology 103 (2003 153-163; Muller-Fuega, A., Biotechnology and Bioengineering, Vol. 84, No. 5, Dec. 5, 2003; Garcia-Sanchez, J. L., Biotechnology and Bioengineering, Vol. 84, No. 5, Dec. 5, 2003; Garcia-Gonzales, M., Journal of Biotechnology, 115 (2005) 81-90. Molina Grima, E., Biotechnology Advances 20 (2003) 491-515).

Photobioreactors can be exposed to one or more light sources to provide microalgae with light as an energy source via light directed to a surface of the photobioreactor. Preferably the light source provides an intensity that is sufficient for the cells to grow, but not so intense as to cause oxidative damage or cause a photoinhibitive response. In some instances a light source has a wavelength range that mimics or approximately mimics the range of the sun. In other instances a different wavelength range is used. Photobioreactors can be placed outdoors or in a greenhouse or other facility that allows sunlight to strike the surface. Preferred photon intensities for species of the genus Porphyridium are between 50 and 300 uE m⁻² s⁻¹ (see for example Photosynth Res. 2005 June; 84(1-3):21-7).

Photobioreactor preferably have one or more parts that allow media entry. It is not necessary that only one substance enter or leave a port. For example, a port can be used to flow culture media into the photobioreactor and then later can be used for sampling, gas entry, gas exit, or other purposes. In some instances a photobioreactor is filled with culture media at the beginning of a culture and no more growth media is infused after the culture is inoculated. In other words, the microalgal biomass is cultured in an aqueous medium for a period of time during which the microalgae reproduce and increase in number; however quantities of aqueous culture medium are not flowed through the photobioreactor throughout the time period. Thus in some embodiments, aqueous culture medium is not flowed through the photobioreactor after inoculation.

In other instances culture media can be flowed though the photobioreactor throughout the time period during which the microalgae reproduce and increase in number. In some instances media is infused into the photobioreactor after inoculation but before the cells reach a desired density. In other words, a turbulent flow regime of gas entry and media entry is not maintained for reproduction of microalgae until a desired increase in number of said microalgae has been achieved, but instead a parameter such as gas entry or media entry is altered before the cells reach a desired density.

Photobioreactors preferably have one or more ports that allow gas entry. Gas can serve to both provide nutrients such as CO₂ as well as to provide turbulence in the culture media. Turbulence can be achieved by placing a gas entry port below the level of the aqueous culture media so that gas entering the photobioreactor bubbles to the surface of the culture. One or more gas exit ports allow gas to escape, thereby preventing pressure buildup in the photobioreactor. Preferably a gas exit port leads to a “one-way” valve that prevents contaminating microorganisms to enter the photobioreactor. In some instances cells are cultured in a photobioreactor for a period of time during which the microalgae reproduce and increase in number, however a turbulent flow regime with turbulent eddies predominantly throughout the culture media caused by gas entry is not maintained for all of the period of time. In other instances a turbulent flow regime with turbulent eddies predominantly throughout the culture media caused by gas entry can be maintained for all of the period of time during which the microalgae reproduce and increase in number. In some instances a predetermined range of ratios between the scale of the photobioreactor and the scale of eddies is not maintained for the period of time during which the microalgae reproduce and increase in number. In other instances such a range can be maintained.

Photobioreactors preferably have at least one port that can be used for sampling the culture. Preferably a sampling port can be used repeatedly without altering compromising the axenic nature of the culture. A sampling port can be configured with a valve or other device that allows the flow of sample to be stopped and started. Alternatively a sampling port can allow continuous sampling. Photobioreactors preferably have at least one port that allows inoculation of a culture. Such a port can also be used for other purposes such as media or gas entry.

Microalgae that produce polysaccharides can be cultured in photobioreactors. Microalgae that produce polysaccharide that is not attached to cells can be cultured for a period of time and then separated from the culture media and secreted polysaccharide by methods such as centrifugation and tangential flow filtration. Preferred organisms for culturing in photobioreactors to produce polysaccharides include Porphyridium sp., Porphyridium cruentum, Porphyridium purpureum, Porphyridium aerugineum, Rhodella maculata, Rhodella reticulata, Chlorella autotrophica, Chlorella stigmatophora, Chlorella capsulata, Achnanthes brevipes and Achnanthes longipes.

C. Non-Microalgal Polysaccharide Production

Organisms besides microalgae can be used to produce polysaccharides, such as lactic acid bacteria (see for example Stinglee, F., Molecular Microbiology (1999) 32(6), 1287-1295; Ruas_Madiedo, P., J. Dairy Sci. 88:843-856 (2005); Laws, A., Biotechnology Advances 19 (2001) 597-625; Xanthan gum bacteria: Pollock, T J., J. Ind. Microbiol. Biotechnol., 1997 August; 19(2):92-7; Becker, A., Appl. Micrbiol. Bioltechnol. 1998 August; 50(2):92-7; Garcia-Ochoa, F., Biotechnology Advances 18 (2000) 549-579., seaweed: Talarico, L B., et al., Antiviral Research 66 (2005) 103-110; Dussealt, J., et al., J Biomed Mater Res A., (2005) Nov. 1; Melo, F. R., J Biol Chem 279:20824-35 (2004)).

D. Ex Vivo Methods

Microalgae and other organisms can be manipulated to produce polysaccharide molecules that are not naturally produced by methods such as feeding cells with monosaccharides that are not produced by the cells (Nature. 2004 Aug. 19; 430(7002):873-7). For example, species listed in Table 1 are grown according to the referenced growth protocols, with the additional step of adding to the culture media a fixed carbon source that is not in the culture media as published and referenced in Table 1 and is not produced by the cells in a detectable amount. In addition, such cells can first be transformed to contain a carbohydrate transporter, thus facilitating the entry of monosaccharides.

E. In vitro methods

Polysaccharides can be altered by enzymatic and chemical modification. For example, carbohydrate modifying enzymes can be added to a preparation of polysaccharide and allowed to catalyze reactions that alter the structure of the polysaccharide. Chemical methods can be used to, for example, modify the sulfation pattern of a polysaccharide (see for example Carbohydr. Polym. 63:75-80 (2000); Pomin V H., Glycobiology. 2005 December; 15(12):1376-85; Naggi A., Semin Thromb Hemost. 2001 October; 27(5):437-43 Review; Habuchi, O., Glycobiology. 1996 January; 6(1); 51-7; Chen, J., J. Biol. Chem. In press; Geresh., S et al., J. Biochem. Biophys. Methods 50 (2002) 179-187.).

F. Polysaccharide Purification Methods

Exopolysaccharides can be purified from microalgal cultures by various methods, including those disclosed herein.

Precipitation: For example, polysaccharides can be precipitated by adding compounds such as cetylpyridinium chloride, isopropanol, ethanol, or methanol to an aqueous solution containing a polysaccharide in solution. Pellets of precipitated polysaccharide can be washed and resuspended in water, buffers such as phosphate buffered saline or Tris, or other aqueous solutions (see for example Farias, W. R. L., et al., J. Biol. Chem. (2000) 275; (38)29299-29307; U.S. Pat. No. 6,342,367; U.S. Pat. No. 6,969,705).

Dialysis: Polysaccharides can also be dialyzed to remove excess salt and other small molecules (see for example Gloaguen, V., et al., Carbohydr Res. 2004 Jan. 2; 339(1):97-103; Microbiol Immunol. 2000; 44(5):395-400.).

Tangential Flow Filtration: Filtration can be used to concentrate polysaccharide and remove salts. For example, tangential flow filtration (TFF), also known as cross-flow filtration, can be used)). For a preferred filtration method see Geresh, Carb. Polym. 50; 183-189 (2002), which discusses use of a MaxCell A/G technologies 0.45 uM hollow fiber filter. Also see for example Millipore Pellicon® devices, used with 100 kD, 300 kD, 1000 kD (catalog number P2C01MC01), 0.1 uM (catalog number P2VVPPV01), 0.22 uM (catalog number P2GVPPV01), and 0.45 uM membranes (catalog number P2HVMPV01). It is preferred that the polysaccharides do not pass through the filter at a significant level. It is also preferred that polysaccharides do not adhere to the filter material. TFF can also be performed using hollow fiber filtration systems.

Non-limiting examples of tangential flow filtration include use of a filter with a pore size of at least about 0.1 micrometer, at least about 0.12 micrometer, at least about 0.14 micrometer, at least about 0.16 micrometer, at least about 0.18 micrometer, at least about 0.2 micrometer, at least about 0.22 micrometer, or at least about 0.45 micrometer. Preferred pore sizes of TFF allow contaminants to pass through but not polysaccharide molecules.

Tangential flow filtration may also be used to isolate and/or purify polysaccharides for further analysis or comparison. In some embodiments, the level of sulfation in polysaccharides from different cells or cell material may be analyzed or compared. In cases of intact cells or homogenized cell material, the cells or material may be resuspended in water or other suitable liquid to allow the release of polysaccharides into the liquid. The release may be augmented by agitation of the cells or material in the liquid, such as by vortexing, or by the use of an elevated temperature, such as a warm liquid. The released polysaccharides may be separated from the cells or insoluble cellular material by any convenient means, like centrifugation or filtration as non-limiting examples. The polysaccharide containing liquid may then be subjected to tangential flow filtration to isolate polysaccharides suitable for further analysis, such as combustion to determine the amount of sulfation in the polysaccharides. The amount of polysaccharide, or sulfation thereof, may be standardized against any known cellular metabolite or component, such as, but not limited to, the amount of DNA per gram of cells or cell material. In alternative embodiments, the polysaccharides from a cell extract may be analyzed as described above. Polysaccharides released into the extract, optionally with agitation and/or an elevated temperature as disclosed herein, may be separated from insoluble cellular material as described above. The soluble material may then be subjected to tangential flow filtration to isolate polysaccharides for further analysis or comparison.

In some embodiments, the amount or level of sulfation in the polysaccharides is analyzed and compared to the amount of sulfates used to culture the microalgae. Thus the amount or level of sulfation in the polysaccharides of cells grown at about 100 mM, about 200 mM, about 300 mM, about 400 mM, about 500 mM, about 600 mM, about 700 mM, or about 800 mM or higher, sulfate may be determined by routine and repetitive methods. Using sulfated exopolysaccharides as a non-limiting example, a disclosed polysaccharide may contain at least about 4.6% sulfur, at least about 4.75% sulfur, at least about 5.0% sulfur, at least about 5.25% sulfur, at least about 5.5% sulfur, at least about 5.75% sulfur, at least about 6.0% sulfur, at least about 6.25% sulfur, at least about 6.5% sulfur, at least about 6.75% sulfur, or at least about 7.0% sulfur by weight.

In further embodiments, the amount or level of sulfur by weight in the polysaccharides of a sample of cells or cell material may determined without knowledge of the amount of sulfate used to culture the cells. The amount or level of sulfur by weight may range from about 3.0 to about 7.0% as described above. In an alternative embodiment, the amount or level of sulfur may be used in a method to determine the amount of sulfates used to culture the cells and produce the cell material. The method may comprise comparison of the amount or level of sulfur, per unit of cellular metabolite or component, in a sample to that of cells grown in known amounts of sulfates. In further embodiments, the method may be used to identify cells, and so polysaccharides, prepared or obtained by methods disclosed herein. For example, homogenized Porphyridium biomass may be incubated in distilled water, subjected to agitation to realease polysaccharide into solution, and centrifuged to remove solids. The resulting soluble polysaccharide can then be purified, concentrated, and diafiltered using methods disclosed in the Examples. The resulting high purity polysaccharide may then be subjected to sulfur content analysis. The sulfur content analysis not only indicated the level of sulfation of the polysaccharide but also can indicate the level of sulfate under which the cells were cultured.

Ion Exchange Chromatography:_Anionic polysaccharides can be purified by anion exchange chromatography. (Jacobsson, I., Biochem J. 1979 Apr. 1; 179(1):77-89; Karamanos, N K., Eur J. Biochem. 1992 Mar. 1; 204(2):553-60).

Protease Treatment: polysaccharides can be treated with proteases to degrade contaminating proteins. In some instances the contaminating proteins are attached, either covalently or noncovalently to polysaccharides. In other instances the polysaccharide molecules are in a preparation that also contains proteins. Proteases can be added to polysaccharide preparations containing proteins to degrade proteins (for example, the protease from Streptomyces griseus can be used (SigmaAldrich catalog number P5147). After digestion, the polysaccharide is preferably purified from residual proteins, peptide fragments, and amino acids. This purification can be accomplished, for example, by methods listed above such as dialysis, filtration, and precipitation.

Heat treatment can also be used to eliminate proteins in polysaccharide preparations (see for example Biotechnol Lett. 2005 January; 27(1):13-8; FEMS Immunol Med. Microbiol. 2004 Oct. 1; 42(2):155-66; Carbohydr Res. 2000 Sep. 8; 328(2):199-207; J Biomed Mater Res. 1999; 48(2):111-6; Carbohydr Res. 1990 Oct. 15; 207(1):101-20).

The invention thus includes production of an exopolysaccharide comprising separating the exopolysaccharide from contaminants after proteins attached to the exopolysaccharide have been degraded or destroyed. The proteins may be those attached to the exopolysaccharide during culture of a microalgal cell in media, which is first separated from the cells prior to proteolysis or protease treatment. The cells may be those of the genus Porphyridium as a non-limiting example.

In one non-limiting example, a method of producing an exopolysaccharide is provided wherein the method comprises culturing cells of the genus Porphyridium; separating cells from culture media; destroying protein attached to the exopolysaccharide present in the culture media; and separating the exopolysaccharide from contaminants. In some methods, the contaminant(s) are selected from amino acids, peptides, proteases, protein fragments, and salts. In other methods, the contaminant is selected from NaCl, MgSO₄, MgCl₂, CaCl₂, KNO₃, KH₂PO₄, NaHCO₃, Tris, ZnCl₂, H₃BO₃, CoCl₂, CuCl₂, MnCl₂, (NH₄)₆Mo₇O₂₄, FeCl3 and EDTA.

Drying Methods: After purification of methods such as those above, polysaccharides can be dried using methods such as lyophilization and heat drying (see for example Shastry, S., Brazilian Journal of Microbiology (2005) 36:57-62; Matthews K H., Int J. Pharm. 2005 Jan. 31; 289(1-2):51-62. Epub 2004 Dec. 30; Gloaguen, V., et al., Carbohydr Res. 2004 Jan. 2; 339(1):97-103).

Tray dryers accept moist solid on trays. Hot air (or nitrogen) is circulated to dry. Shelf dryers can also employ reduced pressure or vacuum to dry at room temperature when products are temperature sensitive and are similar to a freeze-drier but less costly to use and can be easily scaled-up.

Spray dryers are relatively simple in operation, which accept feed in fluid state and convert it into a dried particulate form by spraying the fluid into a hot drying medium.

Rotary dryers operate by continuously feeding wet material, which is dried by contact with heated air, while being transported along the interior of a rotating cylinder, with the rotating shell acting as the conveying device and stirrer.

Spin flash dryers are used for drying of wet cake, slurry, or paste which is normally difficult to dry in other dryers. The material is fed by a screw feeder through a variable speed drive into the vertical drying chamber where it is heated by air and at the same time disintegrated by a specially designed disintegrator. The heating of air may be direct or indirect depending upon the application. The dry powder is collected through a cyclone separator/bag filter or with a combination of both.

Whole Cell Extraction: Intracellular polysaccharides and cell wall polysaccharides can be purified from whole cell mass (see form example U.S. Pat. No. 4,992,540; U.S. Pat. No. 4,810,646; J Sietsma J H., et al., Gen Microbiol. 1981 July; 125(1):209-12; Fleet G H, Manners D J., J Gen Microbiol. 1976 May; 94(1):180-92).

G. Microalgae Homogenization Methods

A pressure disrupter pumps of a slurry through a restricted orifice valve. High pressure (up to 1500 bar) is applied, followed by an instant expansion through an exiting nozzle. Cell disruption is accomplished by three different mechanisms: impingement on the valve, high liquid shear in the orifice, and sudden pressure drop upon discharge, causing an explosion of the cell. The method is applied mainly for the release of intracellular molecules. According to Hetherington et al., cell disruption (and consequently the rate of protein release) is a first-order process, described by the relation: log(Rm/(Rm−R))=K N P72.9. R is the amount of soluble protein; Rm is the maximum amount of soluble protein K is the temperature dependent rate constant; N is the number of passes through the homogenizer (which represents the residence time). P is the operating pressure.

In a ball mill, cells are agitated in suspension with small abrasive particles. Cells break because of shear forces, grinding between beads, and collisions with beads. The beads disrupt the cells to release biomolecules. The kinetics of biomolecule release by this method is also a first-order process. In some embodiments, cell containing samples are cryogenically ball milled in a planetary ball mill (Retsch, PM100) at 10-80 g batch size. The powder is placed in a grinding bowl with eight to ten ¾-inch-diameter stainless steel balls. The sample is cooled repeatedly with liquid nitrogen. The material is milled at 400-550 rpm for 30 to 60 min. The final product is dried in a desiccator overnight.

Another widely applied method is the cell lysis with high frequency sound that is produced electronically and transported through a metallic tip to an appropriately concentrated cellular suspension, ie: sonication. The concept of ultrasonic disruption is based on the creation of cavities in cell suspension.

In some embodiments the invention comprises polysaccharides from a species of the genus Porphyridium, optionally sulfated at a level of 4.7 or higher, that have been reduced in molecular weight through milling or shearing such as, for example, sonication or ball milling.

Blending (high speed or Waring), the french press, or even centrifugation in case of weak cell walls, also disrupt the cells by using the same concepts.

Cells can also be ground after drying in devices such as a colloid mill.

In optional embodiments, the invention includes a method of preparing a dry homogenate by use of lyophilization. As a non-limiting example, a cell containing material is lyophilized prior to homogenization, such as by grinding or milling. In some cases, the cell containing material comprises both cells (biomass) and soluble polysaccharides in the culture medium. In other embodiments, only biomass is used. To prepare a dry material, the cell-containing material is optionally dried (at least partially) or otherwise concentrated before subsequent lyophilization. The lyophilized material may be homogenized to produce a homogenate comprising a powder or particles of a subcellular size range. Non-limiting examples include particles that are less than 800 microns in diameter, less than 200 microns in diameter, less than 100 microns in diameter, less than 50 microns in diameter, less than 10 microns in diameter, less than 5 microns in diameter, and less than 1 micron in diameter. In some embodiments the average particle size of a composition disclosed herein is less than 800 microns in diameter, less than 200 microns in diameter, less than 100 microns in diameter, less than 50 microns in diameter, less than 10 microns in diameter, less than 5 microns in diameter, and less than 1 micron in diameter.

Without being bound by theory, and offered to improve the understanding of the invention, the use of homogenized cells (or biomass) is believed to improve the bioavailability of polysaccharides after administration to a mammal as described herein. As a non-limiting example, the homogenized material may improve exposure of the polysaccharides to solvent and subsequent uptake in the digestive tract of a mammal. Alternatively, the homogenization process may fragment the polysaccharides under certain conditions, such as to produce fragments of sulfated polysaccharides, which increase the total activity available in the homogentate. A further alternative is that the homogenization process releases intracellular polysaccharides which add to the total activity in the homogenate. While none of these proposed mechanisms may be responsible for the improved activity seen with homogenates of cells, a combination of two or more of the above mechanisms also may be responsible for the improved activity.

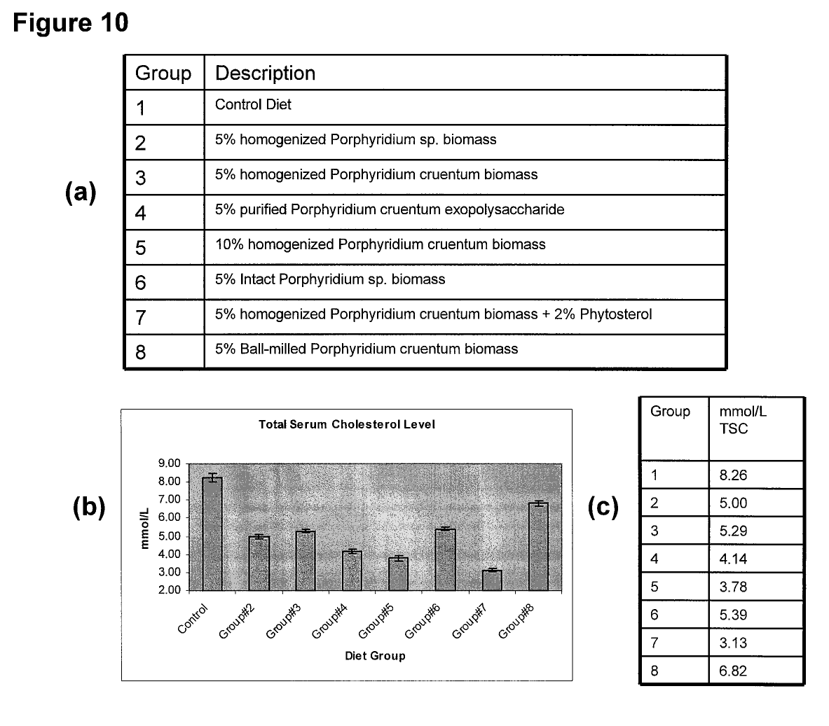

The use of cell material, such as a homogenate, appears to be contrary to earlier reports of improved effects seen with the use of purified polysaccharides compared to unpurified biomass (see Dvir et al. Br. J. Nutri. (2000) 84:469-476; and Ginzberg et al. J. Appl. Phycology (2000) 12:325-330). These earlier reports appear to suggest that biomass is not as effective as purified polysaccharide. But as evident by data disclosed herein, homogenized biomass is effective at reducing total serum cholesterol (TSC), and more so than intact biomass. For example, there is an approximately 8% decrease in TSC between homogenate and intact biomass administered at the same dosage (see FIG. 10 herein, groups 2 vs. 6). In addition, a biomass homogenate at a 5% or 10% diet reduced TSC to a much greater extent (approximately 39% and 54% respectively) than non-homogenized biomass administered at much higher dosages as discussed in the literature (22% reduction in TSC observed for a 19% diet of unpurified biomass—see Dvir et al. Br. J. Nutri. (2000) 84:469-476). As such it was a surprising result to see that lower dosages of a biomass homogenate generate such a significant reduction in TSC.

There is an approximately 8% decrease in total serum cholesterol between homogenized and intact biomass (see FIG. 10, groups 2 vs. 6). In addition, homogenized biomass at a 5% and 10% diet reduced total serum cholesterol to a much greater extent (approximately 39% and 54% respectively) than non-homogenized biomass administered at much higher dosages discussed in the literature (22% reduction in total serum cholesterol for a 19% diet of intact biomass—see Dvir et al. Br. J. Nutri. (2000) 84:469-476). As such it was a surprising result to see that lower dosages of biomass, when processed according to the methods disclosed herein, provide far greater reduction in total serum cholesterol.

Because the percentage of polysaccharide as a function of the dry weight of a microalgae cell can frequently be in excess of 50%, microalgae cell homogenates can be considered partially pure polysaccharide compositions. Cell disruption aids in increasing the amount of solvent-accessible polysaccharide by breaking apart cell walls that are largely composed of polysaccharide.

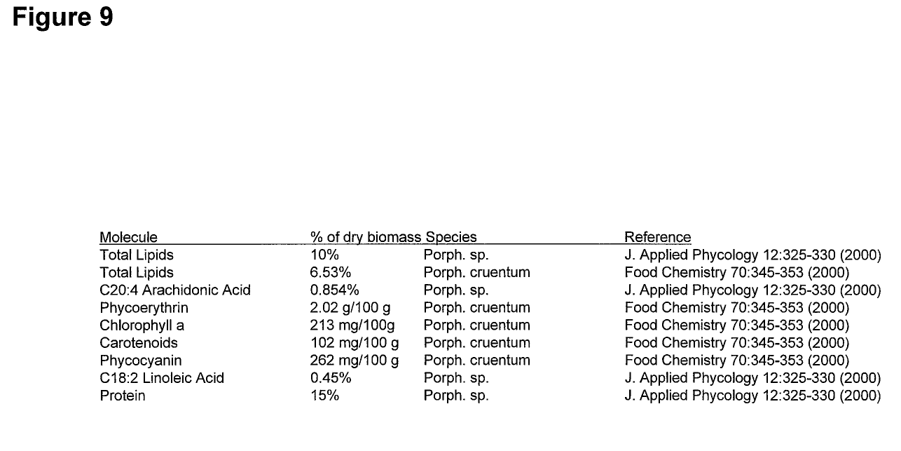

Homogenization as described herein can increase the amount of solvent-available polysaccharide significantly. For example, homogenization can increase the amount of solvent-available polysaccharide by at least a factor of 0.25, at least a factor of 0.5, at least a factor of 1, at least a factor of 2, at least a factor of 3, at least a factor of 4, at least a factor of 5, at least a factor of 8, at least a factor of 10, at least a factor of 15, at least a factor of 20, at least a factor of 25, and at least a factor of 30 or more compared to the amount of solvent-available polysaccharide in an identical or similar quantity of non-homogenized cells of the same type. One way of determining a quantity of cells sufficient to generate a given quantity of homogenate is to measure the amount of a compound in the homogenate and calculate the gram quantity of cells required to generate this amount of the compound using known data for the amount of the compound per gram mass of cells. The quantity of many such compounds per gram of particular microalgae cells are known. For examples, see FIG. 9. Given a certain quantity of a compound in a composition, the skilled artisan can determine the number of grams of intact cells necessary to generate the observed amount of the compound. The number of grams of microalgae cells present in the composition can then be used to determine if the composition contains at least a certain amount of solvent-available polysaccharide sufficient to indicate whether or not the composition contains homogenized cells, such as for example five times the amount of solvent-available polysaccharide present in a similar or identical quantity of unhomogenized cells.

H. Analysis Methods

Assays for detecting polysaccharides can be used to quantitate starting polysaccharide concentration, measure yield during purification, calculate density of secreted polysaccharide in a photobioreactor, measure polysaccharide concentration in a finished product, and other purposes.

The phenol: sulfuric acid assay detects carbohydrates (see Hellebust, Handbook of Psychological Methods, Cambridge University Press, 1978; and Cuesta G., et al., J Microbiol Methods. 2003 January; 52(1):69-73). The 1,6 dimethylmethylene blue assay detects anionic polysaccharides. (see for example Braz J Med Biol Res. 1999 May; 32(5):545-50; Clin Chem. 1986 November; 32(11):2073-6).

Polysaccharides can also be analyzed through methods such as HPLC, size exclusion chromatography, and anion exchange chromatography (see for example Prosky L, Asp N, Schweizer T F, DeVries J W & Furda 1 (1988) Determination of insoluble, soluble and total dietary fiber in food and food products: Interlaboratory study. Journal of the Association of Official Analytical Chemists 71, 1017±1023; Int J Biol Macromol. 2003 November; 33(1-3):9-18)

Polysaccharides can also be detected using gel electrophoresis (see for example Anal Biochem. 2003 Oct. 15; 321(2):174-82; Anal Biochem. 2002 Jan. 1; 300(1):53-68).

Monosaccharide analysis of polysaccharides can be performed by combined gas chromatography/mass spectrometry (GC/MS) of the per-O-trimethylsilyl (TMS) derivatives of the monosaccharide methyl glycosides produced from the sample by acidic methanolysis (see Merkle and Poppe (1994) Methods Enzymol. 230:1-15; York, et al. (1985) Methods Enzymol. 118:3-40).

### III Compositions

A. General

Compositions of the invention include a microalgal polysaccharide or homogenate as described herein. In embodiments relating to polysaccharides, including exopolysaccharides, the composition may comprise a homogenous or a heterogeneous population of polysaccharide molecules, including sulfated polysaccharides as non-limiting embodiments. Non-limiting examples of homogenous populations include those containing a single type of polysaccharide molecule, such as that with the same structure and molecular weight. Non-limiting examples of heterogeneous populations include those containing more than one type of polysaccharide molecule, such as a mixture of polysaccharides having a molecular weight (MW) within a range or a MW above or below a MW value. For example, the Porphyridium sp. exopolysaccharide is typically produced in a range of sizes from 3-5 million Daltons. Of course a polysaccharide containing composition of the invention may be optionally protease treated, or reduced in the amount of protein, as described above.

In some embodiments, a composition of the invention may comprise one or more polysaccharides produced by microalgae that have not been recombinantly modified. The microalgae may be those which are naturally occurring or those which have been maintained in culture in the absence of alteration by recombinant DNA techniques or genetic engineering.

In other embodiments, the polysaccharides are those from modified microalgae, such as, but not limited to, microalgae modified by recombinant techniques. Non-limiting examples of such techniques include introduction and/or expression of an exogenous nucleic acid sequence encoding a gene product; genetic manipulation to decrease or inhibit expression of an endogenous microalgal gene product; and/or genetic manipulation to increase expression of an endogenous microalgal gene product. The invention contemplates recombinant modification of the various microalgae species described herein. In some embodiments, the microalgae is from the genus Porphyridium.

Polysaccharides provided by the invention that are produced by genetically modified microalgae or microalgae that are provided with an exogenous carbon source can be distinct from those produced by microalgae cultured in minimal growth media under photoautotrophic conditions (ie: in the absence of a fixed carbon source) at least in that they contain a different monosaccharide content relative to polysaccharides from unmodified microalgae or microalgae cultured in minimal growth media under photoautotrophic conditions. Non-limiting examples include polysaccharides having an increased amount of arabinose (Ara), rhamnose (Rha), fucose (Fuc), xylose (Xyl), glucuronic acid (GlcA), galacturonic acid (GalA), mannose (Man), galactose (Gal), glucose (Glc), N-acetyl galactosamine (GalNAc), N-acetyl glucosamine (GlcNAc), and/or N-acetyl neuraminic acid (NANA), per unit mass (or per mole) of polysaccharide, relative to polysaccharides from either non-genetically modified microalgae or microalgae cultured photoautotrophically. An increased amount of a monosaccharide may also be expressed in terms of an increase relative to other monosaccharides rather than relative to the unit mass, or mole, of polysaccharide. An example of genetic modification leading to production of modified polysaccharides is transforming a microalgae with a carbohydrate transporter gene, and culturing a transformant in the presence of a monosaccharide which is transported into the cell from the culture media by the carbohydrate transporter protein encoded by the carbohydrate transporter gene. In some instances the culture can be in the dark, where the monosaccharide, such as glucose, is used as the sole energy source for the cell. In other instances the culture is in the light, where the cells undergo photosynthesis and therefore produce monosaccharides such as glucose in the chloroplast and transport the monosaccharides into the cytoplasm, while additional exogenously provided monosaccharides are transported into the cell by the carbohydrate transporter protein. In both instances monosaccharides from the cytoplasm are transported into the endoplasmic reticulum, where polysaccharide synthesis occurs. Novel polysaccharides produced by non-genetically engineered microalgae can also be generated by nutritional manipulation, ie: exogenously providing carbohydrates in the culture media that are taken up through endogenous transport mechanisms. Uptake of the exogenously provided carbohydrates can be induced, for example, by culturing the cells in the dark, thereby forcing the cells to utilize the exogenously provided carbon source. For example, Porphyridium cells cultured in the presence of 7% glycerol in the dark produce a novel polysaccharide because the intracellular carbon flux under these nutritionally manipulated conditions is different from that under photosynthetic conditions. Insertion of carbohydrate transporter genes into microalgae facilitates, but is not strictly necessary for, polysaccharide structure manipulation because expression of such genes can significantly increase the concentration of a particular monosaccharide in the cytoplasm of the cell. Many carbohydrate transporter genes encode proteins that transport more than one monosaccharide, albeit with different affinities for different monosaccharides (see for example Biochimica et Biophysica Acta 1465 (2000) 263-274). In some instances a microalgae species can be transformed with a carbohydrate transporter gene and placed under different nutritional conditions, wherein one set of conditions includes the presence of exogenously provided galactose, and the other set of conditions includes the presence of exogenously provided xylose, and the transgenic species produces structurally distinct polysaccharides under the two conditions. By altering the identity and concentration of monosaccharides in the cytoplasm of the microalgae, through genetic and/or nutritional manipulation, the invention provides novel polysaccharides. Nutritional manipulation can also be performed, for example, by culturing the microalgae in the presence of high amounts of sulfate, as described herein. In some instances nutritional manipulation includes addition of one or more exogenously provided carbon sources as well as one or more other non-carbohydrate culture component, such as 50 mM MgSO₄.

In some embodiments, the increase in one or more of the above listed monosaccharides in a polysaccharide may be from below to above detectable levels and/or by at least about 5%, to at least about 2000%, relative to a polysaccharide produced from the same microalgae in the absence of genetic or nutritional manipulation. Therefore an increase in one or more of the above monosaccharides, or other carbohydrates listed in Tables 2 or 3, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 1000%, at least about 1100%, at least about 1200%, at least about 1300%, at least about 1400%, at least about 1500%, at least about 1600%, at least about 1700%, at least about 1800%, or at least about 1900%, or more, may be used in the practice of the invention.

In cases wherein the polysaccharides from unmodified microalgae do not contain one or more of the above monosaccharides, the presence of the monosaccharide in a microalgal polysaccharide indicates the presence of a polysaccharide distinct from that in unmodified microalgae. Thus using particular strains of Porphyridium sp. and Porphyridium cruentum as non-limiting examples, the invention includes modification of these microalgae to incorporate arabinose and/or fucose, because polysaccharides from two strains of these species do not contain detectable amounts of these monosaccharides (see Example 5 herein). In another non-limiting example, the modification of Porphyridium sp. to produce polysaccharides containing a detectable amount of glucuronic acid, galacturonic acid, or N-acetyl galactosamine, or more than a trace amount of N-acetyl glucosamine, is specifically included in the instant disclosure. In a further non-limiting example, the modification of Porphyridium cruentum to produce polysaccharides containing a detectable amount of rhamnose, mannose, or N-acetyl neuraminic acid, or more than a trace amount of N-acetyl-glucosamine, is also specifically included in the instant disclosure.

Put more generally, the invention includes a method of producing a polysaccharide comprising culturing a microalgae cell in the presence of at least about 0.01 micromolar of an exogenously provided fixed carbon compound, wherein the compound is incorporated into the polysaccharide produced by the cell. In some embodiments, the compound is selected from Table 2 or 3. The cells may optionally be selected from the species listed in Table 1, and cultured by modification, using the methods disclosed herein, or the culture conditions also lusted in Table 1.

The methods may also be considered a method of producing a glycopolymer by culturing a transgenic microalgal cell in the presence of at least one monosaccharide, wherein the monosaccharide is transported by the transporter into the cell and is incorporated into a microalgal polysaccharide.

In some embodiments, the cell is selected from Table 1, such as where the cell is of the genus Porphyridium, as a non-limiting example. In some cases, the cell is selected from Porphyridium sp. and Porphyridium cruentum. Embodiments include those wherein the polysaccharide is enriched for the at least one monosaccharide compared to an endogenous polysaccharide produced by a non-transgenic cell of the same species. The monosaccharide may be selected from Arabinose, Fructose, Galactose, Glucose, Mannose, Xylose, Glucuronic acid, Glucosamine, Galactosamine, Rhamnose and N-acetyl glucosamine.

These methods of the invention are facilitated by use of a transgenic cell expressing a sugar transporter, optionally wherein the transporter has a lower Kₘ for glucose than at least one monosaccharide selected from the group consisting of galactose, xylose, glucuronic acid, mannose, and rhamnose. In other embodiments, the transporter has a lower Kₘ for galactose than at least one monosaccharide selected from the group consisting of glucose, xylose, glucuronic acid, mannose, and rhamnose. In additional embodiments, the transporter has a lower Kₘ for xylose than at least one monosaccharide selected from the group consisting of glucose, galactose, glucuronic acid, mannose, and rhamnose. In further embodiments, the transporter has a lower Kₘ for glucuronic acid than at least one monosaccharide selected from the group consisting of glucose, galactose, xylose, mannose, and rhamnose. In yet additional embodiments, the transporter has a lower Kₘ for mannose than at least one monosaccharide selected from the group consisting of glucose, galactose, xylose, glucuronic acid, and rhamnose. In yet further embodiments, the transporter has a lower Kₘ for rhamnose than at least one monosaccharide selected from the group consisting of glucose, galactose, xylose, glucuronic acid, and mannose. Manipulation of the concentration and identity of monosaccharides provided in the culture media, combined with use of transporters that have a different Kₘ for different monosaccharides, provides novel polysaccharides. These general methods can also be used in cells other than microalgae, for example, bacteria that produce polysaccharides.

In alternative embodiments, the cell is cultured in the presence of at least two monosaccharides, both of which are transporter by the transporter. In some cases, the two monosaccharides are any two selected from glucose, galactose, xylose, glucuronic acid, rhamnose and mannose.

In one non-limiting example, the method comprises providing a transgenic cell containing a recombinant gene encoding a monosaccharide transporter; and culturing the cell in the presence of at least one monosaccharide, wherein the monosaccharide is transported by the transporter into the cell and is incorporated into a polysaccharide of the cell. It is pointed out that transportation of a monosaccharide from the media into a microalgal cell allows for the monosaccharide to be used as an energy source, as disclosed below, and for the monosaccharide to be transported into the endoplasmic reticulum (ER) by cellular transporters. In the ER, polysaccharide production and glycosylation, occurs such that in the presence of exogenously provided monosaccharides, the sugar content of the microalgal polysaccharides change.

In some aspects, the invention includes a novel microalgal polysaccharide, such as from microalgae of the genus Porphyridium, comprising detectable amounts of xylose, glucose, and galactose wherein the molar amount of one or more of these three monosaccharides in the polysaccharide is not present in a polysaccharide of Porphyridium that is not genetically or nutritionally modified. An example of a non-nutritionally and non-genetically modified Porphyridium polysaccharide can be found, for example, in Jones R., Journal of Cellular Comparative Physiology 60; 61-64 (1962). In some embodiments, the amount of glucose, in the polysaccharide, is at least about 65% of the molar amount of galactose in the same polysaccharide. In other embodiments, glucose is at least about 70%, at least about 75%, at least about 80%, at least about 85%, at least about 90%, at least about 95%, at least about 100%, at least about 105%, at least about 110%, at least about 120%, at least about 130%, at least about 140%, at least about 150%, at least about 200%, at least about 250%, at least about 300%, at least about 350%, at least about 400%, at least about 450%, at least about 500%, or more, of the molar amount of galactose in the polysaccharide. Further embodiments of the invention include those wherein the amount of glucose in a microalgal polysaccharide is equal to, or approximately equal to, the amount of galactose (such that the amount of glucose is about 100% of the amount of galactose). Moreover, the invention includes microalgal polysaccharides wherein the amount of glucose is more than the amount of galactose.

Alternatively, the amount of glucose, in the polysaccharide, is less than about 65% of the molar amount of galactose in the same polysaccharide. The invention thus provides for polysaccharides wherein the amount of glucose is less than about 60%, less than about 55%, less than about 50%, less than about 45%, less than about 40%, less than about 35%, less than about 30%, less than about 25%, less than about 20%, less than about 15%, less than about 10%, or less than about 5% of the molar amount of galactose in the polysaccharide.

In other aspects, the invention includes a microalgal polysaccharide, such as from microalgae of the genus Porphyridium, comprising detectable amounts of xylose, glucose, galactose, mannose, and rhamnose, wherein the molar amount of one or more of these five monosaccharides in the polysaccharide is not present in a polysaccharide of non-genetically modified and/or non-nutritionally modified microalgae. In some embodiments, the amount of rhamnose in the polysaccharide is at least about 100% of the molar amount of mannose in the same polysaccharide. In other embodiments, rhamnose is at least about 110%, at least about 120%, at least about 130%, at least about 140%, at least about 150%, at least about 200%, at least about 250%, at least about 300%, at least about 350%, at least about 400%, at least about 450%, or at least about 500%, or more, of the molar amount of mannose in the polysaccharide. Further embodiments of the invention include those wherein the amount of rhamnose in a microalgal polysaccharide is more than the amount of mannose on a molar basis.

Alternatively, the amount of rhamnose, in the polysaccharide, is less than about 75% of the molar amount of mannose in the same polysaccharide. The invention thus provides for polysaccharides wherein the amount of rhamnose is less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% of the molar amount of mannose in the polysaccharide.

In additional aspects, the invention includes a microalgal polysaccharide, such as from microalgae of the genus Porphyridium, comprising detectable amounts of xylose, glucose, galactose, mannose, and rhamnose, wherein the amount of mannose, in the polysaccharide, is at least about 130% of the molar amount of rhamnose in the same polysaccharide. In other embodiments, mannose is at least about 140%, at least about 150%, at least about 200%, at least about 250%, at least about 300%, at least about 350%, at least about 400%, at least about 450%, or at least about 500%, or more, of the molar amount of rhamnose in the polysaccharide.

Alternatively, the amount of mannose, in the polysaccharide, is equal to or less than the molar amount of rhamnose in the same polysaccharide. The invention thus provides for polysaccharides wherein the amount of mannose is less than about 95%, less than about 90%, less than about 85%, less than about 80%, less than about 75%, less than about 70%, less than about 65%, less than about 60%, less than about 60%, less than about 55%, less than about 50%, less than about 45%, less than about 40%, less than about 35%, less than about 30%, less than about 25%, less than about 20%, less than about 15%, less than about 10%, or less than about 5% of the molar amount of rhamnose in the polysaccharide.

In further aspects, the invention includes a microalgal polysaccharide, such as from microalgae of the genus Porphyridium, comprising detectable amounts of xylose, glucose, and galactose, wherein the amount of galactose in the polysaccharide, is at least about 100% of the molar amount of xylose in the same polysaccharide. In other embodiments, rhamnose is at least about 105%, at least about 110%, at least about 120%, at least about 130%, at least about 140%, at least about 150%, at least about 200%, at least about 250%, at least about 300%, at least about 350%, at least about 400%, at least about 450%, or at least about 500%, or more, of the molar amount of mannose in the polysaccharide. Further embodiments of the invention include those wherein the amount of galactose in a microalgal polysaccharide is more than the amount of xylose on a molar basis.

Alternatively, the amount of galactose, in the polysaccharide, is less than about 55% of the molar amount of xylose in the same polysaccharide. The invention thus provides for polysaccharides wherein the amount of galactose is less than about 50%, less than about 45%, less than about 40%, less than about 35%, less than about 30%, less than about 25%, less than about 20%, less than about 15%, less than about 10%, or less than about 5% of the molar amount of xylose in the polysaccharide.

In yet additional aspects, the invention includes a microalgal polysaccharide, such as from microalgae of the genus Porphyridium, comprising detectable amounts of xylose, glucose, glucuronic acid and galactose, wherein the molar amount of one or more of these five monosaccharides in the polysaccharide is not present in a polysaccharide of unmodified microalgae. In some embodiments, the amount of glucuronic acid, in the polysaccharide, is at least about 50% of the molar amount of glucose in the same polysaccharide. In other embodiments, glucuronic acid is at least about 55%, at least about 60%, at least about 65%, at least about 70%, at least about 75%, at least about 80%, at least about 85%, at least about 90%, at least about 95%, at least about 100%, at least about 105%, at least about 110%, at least about 120%, at least about 130%, at least about 140%, at least about 150%, at least about 200%, at least about 250%, at least about 300%, at least about 350%, at least about 400%, at least about 450%, or at least about 500%, or more, of the molar amount of glucose in the polysaccharide. Further embodiments of the invention include those wherein the amount of glucuronic acid in a microalgal polysaccharide is more than the amount of glucose on a molar basis.

In other embodiments, the exopolysaccharide, or cell homogenate polysaccharide, comprises glucose and galactose wherein the molar amount of glucose in the exopolysaccharide, or cell homogenate polysaccharide, is at least about 55% of the molar amount of galactose in the exopolysaccharide or polysaccharide. Alternatively, the molar amount of glucose in the exopolysaccharide, or cell homogenate polysaccharide, is at least about 60%, at least about 65%, at least about 70%, at least about 75%, at least about 80%, at least about 85%, at least about 90%, or at least about 100% of the molar amount of galactose in the exopolysaccharide or polysaccharide.

In yet further aspects, the invention includes a microalgal polysaccharide, such as from microalgae of the genus Porphyridium, comprising detectable amounts of xylose, glucose, glucuronic acid, galactose, at least one monosaccharide selected from arabinose, fucose, N-acetyl galactosamine, and N-acetyl neuraminic acid, or any combination of two or more of these four monosaccharides.

Embodiments of the invention include a composition comprising a intact red microalgae cells or a homogenate of red microalgae cells. Non-limiting examples include a powder or particles as disclosed herein. In some embodiments, the composition further includes a carrier suitable for consumption by a mammal, such as a human. Therefore, embodiments of the invention include a food product, such as a human food product, comprising a homogenate of red microalgae cells. In alternative embodiments, the composition may be a capsule or tablet comprising the homogenate.

Additional embodiments of the invention include compositions wherein the red microalgae cells are of the genus Porphyridium. Non-limiting examples include red microalgae cells that are Porphyridium sp. UTEX 637, or a strain derived from Porphyridium sp. UTEX 637; Porphyridium cruentum UTEX 161, or a strain derived from Porphyridium cruentum UTEX 161; Porphyridium aerugineum, or a strain derived from Porphyridium aerugineum; Porphyridium sordidum, or a strain derived from Porphyridium sordidum; or Porphyridium purpureum, or a strain derived from Porphyridium purpureum.

Further embodiments include compositions wherein the homogenate comprises a sulfated exopolysaccharide, secreted by the red microalgae cells, wherein the polysaccharide in purified form contains at least about 4.6% sulfur, at least about 4.75% sulfur, at least about 5.0% sulfur, at least about 5.25% sulfur, at least about 5.5% sulfur, at least about 5.75% sulfur, at least about 6.0% sulfur, at least about 6.25% sulfur, at least about 6.5% sulfur, at least about 6.75% sulfur, or at least about 7.0% sulfur by weight.

Additional embodiments include compositions wherein the homogenate comprises at least about two, at least about three, at least about four, at least about five, at least about six, at least about seven, at least about eight, at least about nine, at least about 10, at least about 12, at least about 14, at least about 16, at least about 18, or at least about 20 times the amount of solvent-available polysaccharide compared to the quantity of unhomogenized cells (biomass) used to form the homogenate. Stated differently, the homogenate contains from about two, to about 20 or more, times the amount or level of solvent-available polysaccharide compared to the unhomogenized cells needed to generate the microalgal cell homogenate.

Embodiments of the invention also include compositions comprising combinations of the above. In some cases, the composition comprises a homogenate with a sulfated exopolysaccharide secreted by red microalgae cells that contains at least 3.0%, or at least 4.75%, sulfur by weight, and the homogenate contains at least two times the amount of solvent-available polysaccharide as present in a quantity of unhomogenized cells needed to generate the microalgal cell homogenate.

In some embodiments the invention includes mixtures of polysaccharides from Porphyridium as described herein and polysaccharides from a species of Chlorella, such as Chlorella marina, Chlorella salina, Chlorella pyrenoidosa, Chlorella vulgaris, Chlorella anitrata, Chlorella antarctica, Chlorella autotrophica, Chlorella regularis, among others (see World Catalog of Algae, 2.sup.nd Edition, pages 58-74; Miyachi et al. (Eds); 1989; Japan Scientific Societies Press Chlorella minutissima and Chlorella pyrenoidosa, as described in U.S. Pat. Nos. 6,974,576, 6,551,596, 4,831,020, 4,533,548 and 6,977,076.

B. Cholesterol Lowering Compositions

Polysaccharides from microalgae can be formulated for ingestion to achieve a hypocholesterolemic effect. For example, the secreted polysaccharide from Porphyridium sp. can be formulated for administration as a cholesterol lowering agent. Secreted polysaccharides from Porphyridium cruentum, Porphyridium purpureum, Porphyridium aerugineum, Rhodella maculata, Rhodella reticulata, Chlorella autotrophica, Chlorella stigmatophora, Chlorella capsulata, Achnanthes brevipes and Achnanthes longipes can also be formulated for administration as a cholesterol lowering agent. These microalgae are cultured, for example, in photobioreactors in the presence of light, more preferably in the presence of strong light such as 175 μmol photons per square meter per second, for a period of time sufficient for the cells to secrete polysaccharide molecules. Some species, such as those of Chlorella and Porphyridium, can also be cultured in the absence of light and in the presence of a fixed carbon source. In some embodiments, the polysaccharides or polysaccharide material will be from a Porphyridium species, such as one that has been subject to genetic and/or nutritional manipulation to produce polysaccharides with altered monosaccharide content and/or altered sulfation.

Patients in need of cholesterol lowering polysaccharide agents such as polysaccharides are preferably those with total cholesterol above 200 mg/dL, those with LDL Cholesterol above 130 mg/dL, those with HDL Cholesterol less than 40 mg/dL, and those with triglycerides above 150 mg/dL.

The invention also comprises administering to a patient described herein a combination of an algal polysaccharide such as that from a cell of the genus Porphyridium and another compound such as a plant phytosterol or a statin such as Pravachol®, Mevacor®, Zocor®, Lescol®, Lipitor®, Baycol®, Crestor®, and Advicor®. The invention also comprises a method of reducing the side effects of a statin drug comprising lowering the dosage of a statin and administering a polysaccharide produced from microalgae, such as for example the polysaccharide from a cell of the genus Porphyridium. Side effects from statins include nausea, irritability and short temper, hostility, homicidal impulses, loss of mental clarity, amnesia, kidney failure, diarrhea, muscle aching and weakness, tingling or cramping in the legs, inability to walk, sleeping problems, constipation, impaired muscle formation, erectile dysfunction, temperature regulation problems, nerve damage, mental confusion, liver damage and abnormalities, neuropathy, and destruction of CoQ10. The invention also includes administering a polysaccharide produced from microalgae, such as for example the polysaccharide from a cell of the genus Porphyridium, to a patient with total cholesterol of 240 mg/dL or more; to a patient with LDL Cholesterol of 130 to 159 mg/dL, 160 to 189 mg/dL, and 190 mg/dL or higher; and to a patient with triglycerides of 150 to 199 mg/dL, or 200 mg/dL or higher.

In one embodiment, cells of the genus Porphyridium are harvested from culture and homogenized to form a composition for administration to lower cholesterol. Homogenization of the cells provides an increased level of bioavailability of the cell wall polysaccharide compared to intact cells. Homogenization can be performed by methods such as sonication, jet milling, colloid milling, wet grinding, dry grinding, and other methods. A preferred composition for cholesterol reduction is homogenized Porphyridium, wherein the average particle size is less then 300 microns, more preferably less than 200 microns, more preferably less than 100 microns, more preferably less than 50 microns, more preferably less than 25 microns, and more preferably less than 10 microns. In some embodiments the cells are dried before grinding, while in other embodiments homogenization is performed on wet cells, such as sonication. Homogenization of microalgae to increase bioavailability of cell wall polysaccharides can be performed to produce homogenates, also referred to herein as polysaccharide material, of any microalgae, including species from Table 1.

Polysaccharides of the invention may be formulated as a composition for oral consumption, as in a dietary supplement as a non-limiting example. The formulation may be in solid or liquid form. For example, purified lyophilized polysaccharide can be formulated in capsules or tablets. Conventional methods for the preparation of capsules or tablets are known to the skilled person. The methods may include use of pharmaceutically acceptable excipients such as binding agents, fillers, disintegrants, or wetting agents, sweeteners, including, pregelatinised maize starch, polyvinylpyrrolidone, hydroxypropyl methylcellulose, fillers, lactose, microcrystalline cellulose, calcium hydrogen phosphate, lubricants, magnesium stearate, talc, silica, potato starch or sodium starch glycolate, sodium lauryl sulfate, mannitol, lactose, starch, magnesium stearate, polyvinyl pyrollidone, sodium saccharine, cellulose and magnesium carbonate in the formation of a capsule or tablet.

In embodiments involving a capsule, the capsule may be comprise a slow-dissolving polymers. Non-limiting polymers include sodium carboxymethylcellulose, hydroxypropylcellulose, hydroxypropylmethylcellulose and hydroxyethylcellulose. Other preferred cellulose ethers are known to the skilled person (Alderman, Int. J. Pharm. Tech. & Prod. Mfr., 1984, 5(3):1-9). Moreover, the polysaccharide material can be directly encapsulated within a capsule or formed into microspheres that are encapsulated. The formation of microspheres may be by a variety of methods known to the skilled person. As a non-limiting example, the polysaccharide(s) are dispersed in a liquid form, such as in an aqueous solution. The liquid is sprayed onto a core particle, such as a nonpareil composed of sugar and/or starch. This forms a microsphere, which may then be dried, or otherwise processed, before being packaged into capsules.

In embodiments involving a tablet, the polysaccharide material can be formed into a solid tablet, optionally with one or more of the excipients listed above. A tablet may be coated by methods known to the skilled person. Solid oral administration can be formulated to give controlled release of the polysaccharide material.

Polysaccharide material may also be formulated into capsule form as a liquid. The liquid may be any suitably formulated for inclusion in a capsule as known to the skilled person. In some embodiments, the liquid is suitably viscous and does not solvate the capsule to result in leakage from the capsule. The liquid may be a preparation that is a variation of those used in other oral administration, such as those in the form of solutions, syrups, or suspensions, all of which may also be used in the practice of the invention. Such liquid preparations can be prepared by conventional means known to the skilled person with pharmaceutically acceptable additives such as, but not limited to, suspending agents, e.g., sorbitol syrup, cellulose derivatives, or hydrogenated edible fats; emulsifying agents, e.g., lecithin or acacia; non-aqueous vehicles, e.g., almond oil, oily esters, ethyl alcohol, or fractionated vegetable oils; and preservatives, e.g., methyl or propyl-p-hydroxybenzoates or sorbic acid. The preparations can also contain buffer salts, flavoring, coloring, and/or sweetening agents as appropriate.

Alternatively, polysaccharide material can be formulated as a food additive. For example, dried polysaccharide can be resuspended in a food substance such as a salad dressing or another sauce or condiment. Alternatively, the material can be formulated into a processed food item. Non-limiting examples include dried foods, canned foods, bars, and frozen foods. Dried foods include dehydrated foods (which are normally rehydrated before consumption), dry cereals, and crackers as non-limiting examples.

In some embodiments, the polysaccharide material can be formulated into a liquid preparation and for administration as a beverage. Such beverage can be alcoholic, non-alcoholic beverage, carbonated, or a health beverage. Such beverage may comprise one or more of the polysaccharides and/or homogenates described herein as well as, optionally, any one or more of the following: a vitamin, electrolyte substitute, caffeine, an amino acid, minerals, artificial and/or natural sweeteners, milk or dry-milk powder, plant phytosterols, and other additives and preserving agents.

Additional carriers of the invention include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as any two or more of the foregoing in combination.

In some embodiments, the solid or liquid compositions described herein may be advantageously used as a cholesterol lowering composition. Such a composition may comprise 1) a purified microalgal exopolysaccharide or a microalgal cell homogenate (ie: polysaccharide material) and 2) a carrier suitable for human oral consumption as described. The exopolysaccharide or cell homogenate may be produced from cells of the genus Porphyridium as a non-limiting example. As disclosed herein, the exopolysaccharide may be substantially free of protein.

As described herein, embodiments of the invention include a composition comprising a homogenate of red microalgae cells, such as cells of the genus Porphyridium, and at least one additional cholesterol lowering agent or compound. In some embodiments, the agent or compound is selected from a plant phytosterol, a limonoid, a flavonoid, a tocotrienol, or a combination thereof. In additional embodiments, the agent is an inhibitor of HMG CoA reductase, such as a statin.

In embodiments of a composition comprising red microalgae cell material, such as a homogenate, and a plant phytosterol, the weight/weight ratio of cell material (such as homogenate) to plant phytosterol may range from at least about 0.5:1 to at least about 100:1. Additional non-limiting examples include weight/weight ratios of cell material (such as homogenate) to plant phytosterol of at least about 10:1, at least about 20:1, at least about 30:1, at least about 40:1, at least about 50:1, at least about 60:1, at least about 70:1, at least about 80:1, or at least about 90:1.

Phytosterols are steroids derived from a plant, yeast or fungus. Phytosterols have a hydroxyl group at C-3 and no other functional groups. Plant and animal sterols differ. The term phytosterol and/or phytostanol according to the invention, includes analogues of phytosterols, such as those with a double bond at the 5-position in the cyclic unit as in most natural phytosterols, or one or more double bonds at other positions (e.g. 6, 7, 8(9), 8(14), 14, 5/7, or no double bonds in the cyclic unit as in the stanols, or even additional methyl groups as e.g. in alpha-1-sitosterol. The invention also includes the use of a mixture of phytosterol and phytostanol with red microalgae cell material.

Representative examples of a phytosterol and/or phytostanols are those obtained from vegetable oil or wood pulp. Non-limiting examples include alpha-sitosterol, beta-sitosterol, gamma-sitosterol, stigmasterol, ergosterol, campesterol, avenasterol, desmosterol, chalinosterol, poriferasterol, clionasterol, sitostanol, alpha-sitostanol, beta-sitostanol, stigmastanol, campestanol or brassicasterol. In addition to these, at least 44 phytosterols have been identified and they may be used in the practice of the invention as deemed appropriate and/or desirable by the skilled person. Additionally, oryzanol may be used. Phytosterols have been reported and are known in the field (see “Advance in Lipid Research”, pages 193-218, Paoletti, and Kiritchevsky, (Eds) Academic press, NY, (1993); “Effect of Plant Sterols on Lipids and Atherosclerosis”, Pollack, O. J., Pharmac, Ther., 31, 177-208 (1985); Annu Rev Plant Biol. 2004; 55:429-57; Metabolism. 2006 March; 55(3):391-5; Am J Clin Nutr. 2005 June; 81(6):1351-8; Lipids. 2005 February; 40(2):169-74; Lipids. 2003 April; 38(4):367-75; “Efficacy and safety of plant stanols and sterols in the management of blood cholesterol levels.” 2003 Mayo Clin Proc. August; 78(8):965-78; N Engl J. Med. 1995 Nov. 16; 333(20):1308-12; and U.S. Pat. Nos. 6,762,312; 6,979,743; 6,929,816; 6,713,118; and 6,383,514, all of which are hereby incorporated by reference as if fully set forth).

In optional embodiments, the plant phytosterol is a preparation of wood-derived phytosterol. In some embodiments, the wood-derived phytosterol preparation comprises at least about 8% stanol, at least about 40% sitosterol, and at least about 20% campesterol. In other embodiments, the wood-derived phytosterol preparation comprises between about 5% and about 20% stanol, between about 40% and about 60% sitosterol, and between about 15% and about 35% campesterol.

In embodiments of a composition comprising red microalgae cell material (such as a homogenate) and a limonoid, the weight/weight ratio of cell material (such as homogenate) to limonoid may range from at least about 25:1 to at least about 900:1. Additional non-limiting examples include weight/weight ratios of cell material (such as homogenate) to limonoid of at least about 50:1, at least about 100:1, at least about 200:1, at least about 300:1, at least about 400:1, at least about 500:1, at least about 600:1, at least about 700:1, or at least about 800:1. Limonoid compounds are known to the skilled person, and they include limonin or nomilin, which may be present in a composition as described herein.

In embodiments of a composition comprising red microalgae cell material (such as a homogenate) and a flavonoid, the weight/weight ratio of cell material (such as homogenate) to flavonoid may range from at least about 40:1 to at least about 800:1. Additional non-limiting examples include weight/weight ratios of cell material (such as homogenate) to flavonoid of at least about 70:1, at least about 100:1, at least about 200:1, at least about 300:1, at least about 400:1, at least about 500:1, at least about 600:1, or at least about 700:1.

Flavonoid compounds are known to the skilled person, and they include polymethoxylated flavones (pmfs), which decrease apoprotein b (a structural protein needed for endogenous synthesis of LDL and cholesterol). The pmfs tangeretin and nobiletin decrease diacylglycerol acetyl transferase (a liver enzyme needed for endogenous synthesis of triglycerides) activity. Some compositions of the invention comprise a flavonoid selected from hesperidin, naringin, naringenin, hesperitin, nobiletin or tangeretin.

In embodiments of a composition comprising red microalgae cell material (such as a homogenate) and a tocotrienol, the weight/weight ratio of cell material (such as homogenate) to flavonoid may range from at least about 30:1 to at least about 900:1. Additional non-limiting examples include weight/weight ratios of cell material (such as homogenate) to flavonoid of at least about 50:1, at least about 100:1, at least about 200:1, at least about 300:1, at least about 400:1, at least about 500:1, at least about 600:1, at least about 700:1, or at least about 800:1.

Tocotrienol compounds are known to the skilled person, and some have been reported to inhibit HMG CoA reductase, a liver enzyme responsible for endogenous synthesis of cholesterol. A composition of the invention may comprise one or more tocotrienol, like alpha-tocotrienol, gamma tocotrienol, and delta-tocotrienol as non-limiting examples.

Additional embodiments of the invention include a composition comprising a homogenate of red microalgae cells and at least two, or at least three, compounds selected from a plant phytosterol, a limonoid, a flavonoid, and a tocotrienol. In some embodiments, the composition comprises a plant phytosterol and at least one flavonoid selected from hesperidin, naringin, naringenin, hesperitin, nobiletin and tangeretin.

Descriptions of additional limonoid, flavonoid and tocotrienol compounds for inclusion in a composition of the invention or use in a disclosed method are found in U.S. Pat. Nos. 6,239,114; 6,251,400; 6,239,114 and 6,987,125; U.S. patent application Ser. Nos. 09/481,724; 08/938,640; 11/176,593; and 60/560,284; Japanese Patent application PCT/1B98/01721; PCT Patent Application Nos. PCT/1B01/00256; WO02055071; and PCT/US01/08395; Canadian Patent Application No. 304202; and European Patent Application Nos. 98947740.1 and 1920415.5, all of which are hereby incorporated by reference as if fully set forth.

Additional embodiments of the invention include methods of formulating the above described compositions. The method may comprise combining or mixing the homogenate and the one or more cholesterol lowering agent or compound. The homogenate may be prepared as described herein, such as by culturing cells of the genus Porphyridium; separating the cells from culture media to prepare a cell material (biomass in this instance); drying (at least partially) the cell material; and homogenizing/disrupting the cell material. Alternatively, the cells and polysaccharides in the media are isolated together and then dried or concentrated (at least partially) before homogenization.

Additional alternative embodiments of the invention include a kit or composition comprising red microalgae cell material, such as cell material of the genus Porphyridium, and at least one cholesterol lowering component. The combination of cell material and the at least one component is formulated, or packaged, for use or sale as a single unit. In some embodiments, the cell material comprises cells per se, while in other embodiments, the cell material is a polysaccharide containing fraction of a preparation of cells (biomass). The cholesterol lowering component may be an agent or compound selected from a plant phytosterol, a limonoid, a flavonoid, a tocotrienol, or a combination thereof as described herein. In additional embodiments, the agent is an inhibitor of HMG CoA reductase, such as a statin.

In some cases, the composition is formulated to comprise the cell material and the at least one component in contact with each other.

In other cases, the cell material is not in contact with the component as these materials are present together in the form of a kit. In the case of a kit, it may further comprise instructions or a label directing the mixing of the cell material (such as a homogenate) and at least one component in a specific ratio, such as by weight or by mole. Alternatively, the instructions or a label may direct ingestion of the cell material and the at least one component in specific dosages. An additional embodiment of the invention includes instructions or a label directing administration of the cell material and at least one component to lower cholesterol levels in a subject or as part of ongoing treatments or therapies to lower cholesterol levels. A further embodiment of the invention includes instructions or a label directing administration of the cell material and at least one component in a specific order, such as ingestion of the cell material and at least one component over a specified time period or with separation by a specific time period.

Further embodiments of the invention include methods of formulating the above described composition comprising cell material and at least one cholesterol lowering agent or compound. The method may comprise combining or mixing cells, optionally dried (at least partially), and the one or more agent or compound. In some embodiments, the method comprises culturing cells of the genus Porphyridium; separating the cells from culture media to prepare a cell containing material (biomass); drying (at least partially) the cell containing material; and mixing the dried material with at least one cholesterol lowering agent or compound. Non-limiting examples of drying methods include tray drying, spin drying, rotary drying, spin flash drying, and lyophilization.

Optionally, the cell containing material is homogenized before mixing with the at least one agent or compound to generate a mixture. Non-limiting examples of methods for homogenization include pressure disruption, sonication, jet milling and ball milling. In some cases, the cell containing material is homogenized such that the homogenate contains at least two times the amount of solvent-available polysaccharide present in a quantity of unhomogenized cells needed to generate the homogenate. In other cases, the cells are washed before homogenization.

Additional embodiments include methods wherein the cells are cultured in at least 100, at least 200, at least 300, at least 400, at least 500, at least 600, or at least 700 mM sulfate. The sulfate may be provided in at least one form selected from Na₂SO₄.10H₂O, MgSO₄.7H₂O, MnSO₄, and CuSO₄. Where such cells are homogenized before use, the resultant homogenate may contain sulfated exopolysaccharides with at least about 4.6% sulfur by weight of purified polysaccharide, at least about 4.75% sulfur by weight of purified polysaccharide, at least about 5.0% sulfur by weight of purified polysaccharide, at least about 5.25% sulfur by weight of purified polysaccharide, at least about 5.5% sulfur by weight of purified polysaccharide, at least about 5.75% sulfur by weight of purified polysaccharide, at least about 6.0% sulfur by weight of purified polysaccharide, at least about 6.25% sulfur by weight of purified polysaccharide, at least about 6.5% sulfur by weight of purified polysaccharide, at least about 6.75% sulfur by weight of purified polysaccharide, or at least about 7.0% sulfur by weight by weight of purified polysaccharide.

The method of preparing the composition may further comprise adding a carrier suitable for human consumption, or a carrier for oral consumption or administration to a mammal, into the mixture of cell material and at least one cholesterol lowering agent or compound.

C. Administration and Methods of Lowering Cholesterol

The cholesterol lowering compositions of the invention may be administered to a subject in need thereof by any appropriate means. Subjects in need of lower cholesterol levels include human beings, who may be tested for serum or plasma cholesterol levels as commonly practiced in clinical medicine by the skilled person. Based on such tests, an elevated cholesterol level in need of lowering may be identified and treated by the methods of the invention. In some embodiments, the cholesterol to be lowered is that of low density lipoprotein (LDL) in serum. In other embodiments, the cholesterol to be lowered is that of Lp(a), a genetic variation of plasma LDL.

The invention includes a method of lowering cholesterol, said method comprising administering a polysaccharide, as disclosed herein, produced by microalgae. In some embodiments, the administering is oral, optionally with a biologically acceptable carrier.

In some embodiments, the polysaccharide is produced by microalgae selected from Table 1. In some embodiments, the polysaccharide is produced by microalgae of the genus Porphyridium. The administered polysaccharide may be a component of a food composition as a non-limiting example. In one range of embodiments, the amount of polysaccharide administered to a human is from about 0.1 to about 50 grams per day. Additional ranges of the invention include an amount of polysaccharide from about 0.25 to about 50 grams per day, about 0.5 to about 5 grams per day, about 0.75 to about 4 grams per day, or about 1 to about 3 grams per day.

Alternatively, the polysaccharide is administered via a composition comprising a homogenate of red microalgae cells as described herein. The amount of the composition may be that which contains sufficient homogenate to provide the equivalent amount of polysaccharide as described above.

Other embodiments of the invention include a method of lowering cholesterol levels in a subject, such as a human patient, by administering a composition comprising a homogenate of red microalgae cells as described herein. In some cases, the method may comprise oral administration of the composition. The amount of the composition to administer, and be sufficient (or effective) to lower serum cholesterol levels, may be determined by the skilled person by routine and/or repetitive methods. In some embodiments, the amount of homogenate administered is in the range of about 0.005 to about 5 grams per kilogram of mammalian body weight per day. Additional non-limiting examples of amounts to use include about 0.01, about 0.025, about 0.05, about 0.075, about 0.1, about 0.25, about 0.5, about 0.75, about 1, about 1.5, about 2, about 2.5, about 3, about 3.5, about 4, or about 4.5 grams per kg of body weight per day.

In some embodiments, the composition comprises at least one cholesterol lowering agent or compound, such as, but not limited to, a plant phytosterol, a limonoid, a flavonoid, a tocotrienol, or a combination thereof. In embodiments comprising a limonoid, the administered amount thereof may be in the range of about 1 to about 500 mg/day, such as about 10, about 20, about 50, about 100, about 150, about 200, about 250, about 300, about 350, about 400, or about 450 mg/day.

In embodiments comprising a flavonoid, the administered amount thereof may be in the range of about 200 to about 5000 mg/day, such as about 250, about 300, about 350, about 400, about 450, about 500, about 600, about 700, about 800, about 900, about 1000, about 1500, about 2000, about 2500, about 3000, about 3500, about 4000, or about 4500 mg/day. In embodiments comprising a tocotrienol, the administered amount thereof may be in the range of about 1 to about 1200 mg/day, such as about 10, about 20, about 50, about 100, about 150, about 200, about 250, about 300, about 350, about 400, about 450, about 500, about 600, about 700, about 800, about 900, about 1000, or about 1100 mg/day.

### IV Cosmeceutical Compositions and Topical Application

A. General

Compositions, comprising polysaccharides, whole cell extracts, or mixtures of polysaccharides and whole cell extracts, are provided for topical application or non-systemic administration. The polysaccharide may be any one or more of the microalgal polysaccharides disclosed herein, including those produced by a species, or a combination of two or more species, in Table 1. Similarly, a whole cell extract may be that prepared from a microalgal species, or a combination of two or more species, in Table 1. In some embodiments, polysaccharides, such as exopolysaccharides, and cell extracts from microalgae of the genus Porphyridium are used in the practice of the invention. A composition of the invention may comprise from between about 0.001% and about 100%, about 0.01% and about 90%, about 0.1% and about 80%, about 1% and about 70%, about 2% and about 60%, about 4% and about 50%, about 6% and about 40%, about 7% and about 30%, about 8% and about 20%, or about 10% polysaccharide, cell extract, by weight.

Topical compositions are usually formulated with a carrier, such as in an ointment or a cream, and may optionally include a fragrance. One non-limiting class of topical compositions is that of cosmeceuticals. Other non-limiting examples of topical formulations include gels, solutions, impregnated bandages, liposomes, or biodegradable microcapsules as well as lotions, sprays, aerosols, suspensions, dusting powder, impregnated bandages and dressings, biodegradable polymers, and artificial skin. Another non-limiting example of a topical formulation is that of an ophthalmic preparation. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water.

In some embodiments, the polysaccharides contain fucose moieties. In other embodiments, the polysaccharides are sulfated, such as exopolysaccharides from microalgae of the genus Porphyridium. In some embodiments, the polysaccharides will be those from a Porphyridium species, such as one that has been subject to genetic and/or nutritional manipulation to produce polysaccharides with altered monosaccharide content and/or altered sulfation.

In additional embodiments, a composition of the invention comprises a microalgal cell homogenate and a topical carrier. In some embodiments, the homogenate may be that of a species listed in Table 1 or may be material produced by a species in the table.

In further embodiments, a composition comprising purified microalgal polysaccharide and a carrier suitable for topical administration also contains a fusion (or chimeric) protein associated with the polysaccharide. In some embodiments, the fusion protein comprises a first protein, or polypeptide region, with at least about 60% amino acid identity with the protein of SEQ ID NO: 28. In other embodiments, the first protein has at least about 70%, at least about 75%, at least about 80%, at least about 85%, at least about 90%, at least about 95%, or at least about 98%, or higher, amino acid identity with the sequence of SEQ ID NO:28.

The fusion protein may comprise a second protein, or polypeptide region, with a homogenous or heterologous sequence. Non-limiting examples of the second protein include an antibody and an enzyme. In optional embodiments, the enzyme is superoxide dismutase, such as that has at least about 60% amino acid identity with the sequence of SEQ ID NO: 14, SEQ ID NO: 15, and a superoxide dismutase selected from Table 16 as non-limiting examples. In some embodiments, the superoxide dismutase has at least about 70%, at least about 75%, at least about 80%, at least about 85%, at least about 90%, at least about 95%, or at least about 98%, or higher, amino acid identity with the sequences of SEQ ID NO:14 or 15 or from Table 16.

In other embodiments, the second protein is an antibody. Non-limiting examples of antibodies for use in this aspect of the invention include an antibody that selectively binds to an antigen from a pathogen selected from HIV, Herpes Simplex Virus, gonorrhea, Chlamydia, Human Papillomavirus, and Trichomoniasis. In some embodiments, the antibody is a humanized antibody.

B. Methods of Formulation

Polysaccharide compositions for topical application can be formulated by first preparing a purified preparation of polysaccharide. As a non-limiting example, the polysaccharide from aqueous growth media is precipitated with an alcohol, resuspended in a dilute buffer, and mixed with a carrier suitable for application to human skin or mucosal tissue, including the vaginal canal. Alternatively, the polysaccharide can be purified from growth media and concentrated by tangential flow filtration or other filtration methods, and formulated as described above. Intracellular polysaccharides can be also formulated in a similar or identical manner after purification from other cellular components.

As a non-limiting example, the invention includes a method of formulating a cosmeceutical composition, said method comprising culturing microalgal cells in suspension under conditions to allow cell division; separating the microalgal cells from culture media, wherein the culture media contains exopolysaccharide molecules produced by the microalgal cells; separating the exopolysaccharide molecules from other molecules present in the culture media; homogenizing the microalgal cells; and adding the separated exopolysaccharide molecules to the cells before, during, or after homogenization. In some embodiments, the microalgal cells are from the genus Porphyridium.

Examples of polysaccharides, both secreted and intracellular, that are suitable for formulation with a carrier for topical application are listed in Table 1.

Examples of carriers suitable for formulating polysaccharide are described above. Ratios of homogenate:carrier are typically in the range of about 0.001:1 to about 1:1 (volume:volume), although the invention comprises ratios outside of this range, such as, but not limited to, about 0.01:1 and about 0.1:1.

Microalgal cellular extracts can also be formulated for topical administration. It is preferable but not necessary that the cells are physically or chemically disrupted as part of the formulation process. For example, cells can be centrifuged from culture, washed with a buffer such as 1.0 mM phosphate buffered saline, pH 7.4, and sonicated. Preferably the cells are sonicated until the cell walls have been substantially disrupted, as can be determined under a microscope. For example, Porphyridium sp. cells can be sonicated using a Misonix sonicator as described in Example 15.

Cells can also be dried and ground using means such as mortar and pestle, colloid milling, ball milling, or other physical method of breaking cell walls.

After cell disruption, cell homogenate can be formulated with carrier and fragrance as described above for polysaccharides.

C. Co-Administered Compositions

Topical compositions can comprise a portion of a complete composition sold as a single unit. Other portions of the complete compositions can comprise an oral supplement intended for administration as part of a regime for altering skin appearance. Because the top layers of the skin contain dead cells, nutrients delivered via capillaries cannot reach the outer layers of cells. The outer layers of cells must be provided with nutrients though topical administration. However, topical administration is not always an effective method of providing nutrients to deep layers of skin that contain living cells. The compositions provided herein comprise both topical compositions that contain algal polysaccharides and/or cellular extracts as well as oral compositions comprising nutraceutical molecules such as purified polysaccharides, whole cell extracts, carotenoids, polyunsaturated fatty acids, and other molecules that are delivered to the skin via capillaries. The combined effect of the topical and oral administration of these molecules and extracts provides a benefit to skin health that is additive or synergistic compared to the use of only a topical or only an orally delivered product.

Examples of the topical components of the composition include exopolysaccharide from Porphyridium cruentum, Porphyridium sp., list others. Other components of the topical composition can include polysaccharides and/or cell extracts from species listed in Table 1.

Cellular extracts for topical administration can also include cellular homogenates from microalgae that have been genetically engineered. For example, homogenates of Porphyridium sp. that have been engineered to express an exogenous gene encoding superoxide dismutase can be formulated for topical administration. Other genes that can be expressed include carotenoid biosynthesis enzymes and polyunsaturated fatty acid biosynthesis enzymes.

Examples of compositions for oral administration include one or more of the following: DHA, EPA, ARA, lineoileic acid, lutein, lycopene, beta carotene, braunixanthin, zeaxanthin, astaxanthin, linoleic acid, alpha carotene, vitamin C and superoxide dismutase. Compositions for oral administration usually include a carrier such as those described above. Oral compositions can be formulated in tablet or capsule form. Oral compositions can also be formulated in an ingestible form such as a food, tea, liquid, etc.

In a preferred embodiment, at the topical composition and the oral composition both contain at least one molecule in common. For example, the topical composition contains homogenate of Porphyridium cells that contain zeaxanthin, and the oral composition contains zeaxanthin. In another embodiment, the topical composition contains homogenate of Porphyridium cells that contain polysaccharide, and the oral composition contains polysaccharide purified from Porphyridium culture media.

The compositions described herein are packaged for sale as a single unit. For example, a unit for sale comprises a first container holding a composition for topical administration, a second container holding individual doses of a composition for oral administration, and optionally, directions for co-administration of the topical and oral composition.

Some embodiments of the invention include a combination product comprising 1) a first topical composition comprising a microalgal extract or other composition and a carrier suitable for topical application to skin; and 2) a second composition comprising at least one compound in common with the first composition and optionally a carrier suitable for human oral consumption; wherein the first and second compositions are packaged for sale as a single unit. Thus the invention includes co-packaging of the two compositions, optionally with a instructions and/or a label indicating the identity of the contents and/or their proper use.

In some embodiments the first topical composition is selected from an algae extract, optionally from a species selected from Table 1, a vitamin C preparation (such as L-ascorbic acid or ascorbyl palmitate), and a vitamin A preparaion (such as d-alpha-tocopherol). In some embodiments the second oral compositions is selected from an algae extract, optionally from a species selected from Table 1, a vitamin C composition (such as L-ascorbic acid or ascorbyl palmitate), a vitamin A preparaion (such as d-alpha-tocopherol), a polyunsaturated fatty acid (such as Eicosapentaenoic Acid (EPA) or Docosahexaenoic Acid (DHA), zeaxanthin, lutein, beta carotene, and a coenzyme Q preparation (such as ubiquinol-10). Preferred algae extracts contain at least a subset of vitamin C, vitamin A, polyunsaturated fatty acids such as EPA or DHA, and carotenoids such as zeaxanthin, lutein, beta carotene, and cofactors such as coenzyme Q.

D. Methods of Cosmetic Enhancement

In a further aspect, the invention includes a polysaccharide composition suitable for injection into skin to improve its appearance. In some embodiments, the injection is made to alleviate or eliminate wrinkles. In other embodiments, the treatment reduces the visible signs of aging and/or wrinkles.

As known to the skilled person, human skin, as it ages, gradually loses skin components that keep skin pliant and youthful-looking. The skin components include collagen, elastin, and hyaluronic acid, which have been the subject of interest and use to improve the appearance of aging skin.

The invention includes compositions of microalgal polysaccharides, microalgal cell extracts, and microalgal cell homogenates for use in the same manner as collagen and hyaluronic acid. In some embodiments, the polysaccharides will be those of from a Porphyridium species, such as one that has been subject to genetic and/or nutritional manipulation to produce polysaccharides with altered monosaccharide content and/or altered sulfation. In some embodiments, the polysaccharides are formulated as a fluid, optionally elastic and/or viscous, suitable for injection. The compositions may be used as injectable dermal fillers as one non-limiting example. The injections may be made into skin to fill-out facial lines and wrinkles. In other embodiments, the injections may be used for lip enhancement. These applications of polysaccharides are non-limiting examples of non-pharmacological therapeutic methods of the invention.

In further embodiments, the microalgal polysaccharides, cell extracts, and cell homogenates of the invention may be co-formulated with collagen and/or hyaluronic acid (such as the Restylane® and Hylafom® products) and injected into facial tissue. Non-limiting examples of such tissue include under the skin in areas of wrinkles and the lips. In a preferred embodiment, the polysaccharide is substantially free of protein. The injections may be repeated as deemed appropriate by the skilled practitioner, such as with a periodicity of about three, about four, about six, about nine, or about twelve months. In another preferred embodiment, a hyaluronic acid material is mixed with a polysaccharide from the genus Porphyridium prior to co-administration. The invention in this particular embodiment provides longer half-life to the hyaluronic acid due to the potent inhibition of hyaluronidase by polysaccharides isolated from microalgae from the genus Porphyridium. This allows for less injections to a patent.

Thus the invention includes a method of cosmetic enhancement comprising injecting a polysaccharide produced by microalgae into mammalian skin. The injection may be of an effective amount to produce a cosmetic improvement, such as decreased wrinkling or decreased appearance of wrinkles as non-limiting examples. Alternatively, the injection may be of an amount which produces relief in combination with a series of additional injections. In some methods, the polysaccharide is produced by a microalgal species, or two or more species, listed in Table 1. In one non-limiting example, the microalgal species is of the genus Porphyridium and the polysaccharide is substantially free of protein.

### V Compositions for Non-Systemic Administration of Polysaccharides

A. General

Compositions for non-systemic administration include those formulated for localized administration with little or slow release to other parts of a treated subject's body. Non-limiting examples of non-systemic administration include intravaginal application such as via a suppository, cream or foam; injection into a joint between bones; intravitreous or intraocular administration; and rectal administration via suppository, irrigation or other suitable means. In some embodiments, the composition is formulated for the treatment of sexually transmitted diseases, such as those caused by viral agents.

Polysaccharides from microalgae provided herein posses potent antiviral activity (see references cited in Table 1). In additional embodiments, polysaccharides with lubricant properties (see for example Porphyridium and Chlorella polysaccharides) are used in the practice of certain aspects of the invention. These polysaccharides may be formulated in solutions that are added to prophylactic devices. Moreover, the polysaccharides may be one or more described herein, optionally sulfated. In many embodiments, the polysaccharide is produced by a microalgal species, or two or more species, listed in Table 1. In some embodiments, the microalgae is Porphyridium sp. or Porphyridium cruentum.

Thus, the invention includes a sexually transmitted disease prevention composition, said composition comprising 1) a solution comprising a polysaccharide produced from microalgae; and 2) a prophylactic device. In some embodiments, the solution and device are kept separate, but packaged together as a single unit for sale. The solution may be applied to the device by the end user before actual use. Alternatively the solution and device are packaged so that the solution is in direct contact with the device. The prophylactic devices include, but are not limited to, condoms, sponges, and diaphragms.

In some embodiments, the devices are packaged with a lubricant. In other embodiments, the polysaccharide acts as a lubricant and so no other lubricant is needed. In such embodiments, the substance in the composition providing a lubricant function and the substance in the composition providing antiviral activity are the same substance. Alternatively, a combination of a lubricant, such as a cream or lotion, with the polysaccharide of the invention may be used.

In some embodiments, the polysaccharide is in a composition with a carrier used with a prophylactic device described above. Non-limiting examples of a carrier include a spermicide and a lubricant. In other embodiments of the invention, a triple composition, comprising spermicide, lubricant and the polysaccharide, may be used.

In further embodiments, the polysaccharide is associated with a fusion (or chimeric) protein comprising a first protein (or polypeptide region) with at least about 60% amino acid identity with the protein of SEQ ID NO: 28. In some cases, the first protein has at least about 70%, at least about 75%, at least about 80%, at least about 85%, at least about 90%, at least about 95%, or at least about 98%, or higher, amino acid identity with the sequence of SEQ ID NO:28, and further wherein the fusion protein has the ability to specifically bind to the polysaccharide, preferably with high affinity.

The fusion protein may comprise a second protein, or polypeptide region, with a homogenous or heterologous sequence. One non-limiting example of the second protein is an antibody. In some embodiments, the antibody is selective for binding to an antigen of a pathogen, or opportunistic organism, involved in a sexually transmitted disease. Non-limiting examples of antibodies include those that bind an antigen from a pathogen selected from HIV, Herpes Simplex Virus, gonorrhea, Chlamydia, Human Papilloma Virus, and Trichomoniasis.

In other embodiments, the compositions are formulated for improving joint lubrication or treating joint disorders. As described above, microalgal polysaccharides may be used in the same manner as, or in combination with, hyaluronic acid in some compositions of the invention. Hyaluronic acid, or hyaluronan, is used to lubricate joints, such as in viscosupplementation. As a non-limiting example, SYNVISC® (Genzyme Corporation) is an FDA-approved agent which is injected into knee joints to provide lubrication. The elastic and viscous nature of the fluid allows it to function in absorbing shock and improve proper knee movement and flexibility. Other similar hyaluronic acid products include Hylartil®, Arthrease®, Orthovisc® and Artval®. In another preferred embodiment, a hyaluronic acid material is mixed with a polysaccharide from the genus Porphyridium prior to injection into a mammalian joint. The invention in this particular embodiment provides longer half-life to the hyaluronic acid due to the potent inhibition of hyaluronidase by polysaccharides isolated from microalgae from the genus Porphyridium. This allows for less injections to a patent.

Microalgal polysaccharides of the invention are also formulated as fluids with elastic and/or viscous properties such that they may serve as replacements for normal joint fluid. Polysaccharides from the red microalgae Porphyidium sp. have desirable load bearing and shear properties. Polysaccharides with average molecular weights of about 2 to about 7 megadaltons in solution have been found to have very low coefficients of friction (μ<0.01) at low compressions, and increasing only to μ=0.015 at 10 MPa. The low friction, and resistance under high pressure make the polysaccharides highly suitable for biolubrication, such as in human joint lubrication. Advantageously, the polysaccharides are not degraded by hyaluronidase, which degrades hyaluronic acid; are resistant to elevated temperatures; and are anti-inflammatory and anti-irritating. See for example, Golan et al., “Characterization of a Superior Bio-Lubricant Extracted from a Species of Red Microalga” The 39^{th} Annual Meeting of the Israel Society for Microscopy, Ben Gurion University, May 19, 2005, Poster Abstracts (at www.technion.ac.il/technion/materials/ism/ISM2005_posters_abstracts.html); and Gourdon et al. “Superlubricity of a natural polysaccharide from the alga Porphyridium sp.” Abstract Submitted for the March 2005 Meeting of The American Physical Society, Abstract V31.00010 (at absimage.aps.org/image/MWS_MAR05-2004-006269.pdf).

B. Methods of Use

The polysaccharides of the invention may be used in the same or a similar manner. In some embodiments, the polysaccharides will be those from a Porphyridium species, such as one that has been subject to genetic and/or nutritional manipulation to produce polysaccharides with altered monosaccharide content. In some embodiments, a fluid containing one or more polysaccharides is injected into a joint to alleviate joint pain, such as, but not limited to, arthritis and osteoarthritis. Non-limiting examples of joint pain include pain of the knee, shoulder, elbow, and wrist joints. Subjects afflicted with, suffering from, or having joint pain may be diagnosed and/or identified by a skilled person in the field using any suitable method. Non-limiting examples include signs of inflammation, like swelling, pain, or redness; excess fluid in the joint; the need for physical therapy; pain during exercise.

In other embodiments, the polysaccharides of the invention, whether used alone or in combination with hyaluronic acid, are used after the failure, or ineffectiveness, of non-drug treatments or drug therapy for joint pain. Non-limiting examples of non-drug treatments that may be ineffective include avoidance of activities that cause the joint pain, exercise, physical therapy, and removal of excess fluid. Non-limiting examples of drug therapy that may be ineffective include pain relievers, such as acetaminophen and narcotics; anti-inflammatory agents, such as aspirin and other nonsteroidal anti-inflammatory drugs (NSAIDs) such as ibuprofen and naproxen; and injection of steroids.

The invention includes a method of mammalian joint lubrication. Mammalian joint lubrication is used to treat conditions such as osteoarthritis, joint trauma, rheumatoid arthritis, and other degenerative conditions affecting the mammalian joint. Mammalian joints include knees, hips, ankles, shoulders, and other joints. The method comprises injecting a micro al gal polysaccharide of the invention into a cavity containing synovial fluid. The injection may be of an effective amount to produce relief from one or more symptoms of joint pain or discomfort that is alleviated by joint lubrication. Alternatively, the injection may be of an amount which produces relief in combination with a series of additional injections. In some methods, the polysaccharide is produced by a microalgal species, or two or more species, listed in Table 1. In one non-limiting example, the microalgal species is of the genus Porphyridium.

In further embodiments, the methods may also comprise treatment with one or more of the non-drug treatments or drug therapies described herein. As a non-limiting example, injection of a joint lubricating composition of the invention may be combined with administration of an anti-inflammatory agent and optionally physical therapy.

For systemic administration, polysaccharides can be formulated with carriers, excipients, and other compounds. pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d.alpha-tocopherol polyethyleneglycol 1000 succinate, or other similar polymeric delivery matrices or systems, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-beta-cyclodextrins, or other solublized derivatives may also be advantageously used to enhance delivery of therapeutically-effective plant essential oil compounds of the present invention.

The polysaccharide compositions of this invention may be administered orally, parenterally, by inhalation spray, topically, rectally, nasally, buccally, vaginally or via an implanted reservoir, however, oral administration or administration by injection is preferred. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intrasynovial, intrasternal, intrathecal, intralesional and intracranial injection or infusion techniques.

The polysaccharide compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringers solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

Sterile injectable polysaccharide compositions preferably contain less than 1% protein as a function of dry weight of the composition, more preferably less than 0.1% protein, more preferably less than 0.01% protein, less than 0.001% protein, less than 0.0001% protein, more preferably less than 0.00001% protein, more preferably less than 0.000001% protein.

The polysaccharide compositions of the present invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added.

The polysaccharide compositions of the present invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

### VI Food Additive and Nutraceutical Compositions

A. Food Compositions

The invention further provides for the use of polysaccharides as food additives. In some embodiments, the polysaccharides are used to aid in keeping food products homogenous and/or thicken food products, such as in a manner analogous to various gums (e.g. xanthan gum or gum Arabic) known to the skilled person. In these contexts, the polysaccharides act as a hydrocolloid polysaccharide and may be used in the same manner as other hydrocolloid polysaccharides. See for example U.S. Pat. No. 5,126,158. In other embodiments, the polysaccharides are used to stabilize or emulsify foods. These uses of the polysaccharides are based upon their Theological properties, which are similar or superior to those of previously used gums and additives. In some embodiments, the polysaccharides will be those of from a Porphyridium species, such as one that has been subject to genetic and/or nutritional manipulation to produce polysaccharides with altered monosaccharide content and/or altered sulfation.

The methods of the invention thus include a method comprising addition of one or more polysaccharides of the invention to a food product. The addition may be for use as a thickener or an emulsifier, such as to keep foods homogenous. In some embodiments, the addition is to a beverage product, such as to thicken it or improve its texture, appearance, or “feel” upon consumption. In other embodiments, the method stabilizes and/or emulsifies a food composition or product. The method may comprise adding one or more microalgal polysaccharide, as described herein, to the food composition or product. Non-limiting examples include polysaccharides produced by microalgae described herein. In some instances, the microalgae is of the genus Porphyridium.

In some embodiments, a microalgal cell material (such as a homogenate) of the invention may be incorporated into a food, a food supplement, a liquid, a beverage, an emulsion, a powder, or mixtures thereof for ingestion by a mammal. In some cases, the cell material is formulated as a composition for addition to another food substance, such as for sprinkling, or spreading, on a salad, bread, or cereal as non-limiting examples. In further embodiments, the cell material may be incorporated into a cereal product, such as a ready-to-eat variety; incorporated into a baked product, such as a bread, muffin, or waffle; incorporated into a pasta, healthy dessert, snack (such as an athletic bar or healthy drink as non-limiting examples) or a high fiber food.

B. Nutraceuticals

In another aspect, the invention includes nutraceutical compositions comprising one or more polysaccharides, or microalgal cell extract or homogenate, of the invention. A nutraceutical composition serves as a nutritional supplement upon consumption. In other embodiments, a nutraceutical may be bioactive and serve to affect, alter, or regulate a bioactivity of an organism.

A nutraceutical may be in the form of a solid or liquid formulation. In some embodiments, a solid formulation includes a capsule or tablet formulation as described above. In other embodiments, a solid nutraceutical may simply be a dried microalgal extract or homogenate, as well as dried polysaccharides per se. In liquid formulations, the invention includes suspensions, as well as aqueous solutions, of polysaccharides, extracts, or homogenates.

The methods of the invention include a method of producing a nutraceutical composition. Such a method may comprise drying a microalgal cell homogenate or cell extract. The homogenate may be produced by disruption of microalgae which has been separated from culture media used to propagate (or culture) the microalgae Thus in one non-limiting example, a method of the invention comprises culturing red microalgae; separating the microalgae from culture media; disrupting the microalgae to produce a homogenate; and drying the homogenate. In similar embodiments, a method of the invention may comprise drying one or more polysaccharides produced by the microalgae.

In some embodiments, a method of the invention comprises drying by tray drying, spin drying, rotary drying, spin flash drying, or lyophilization. In other embodiments, methods of the invention comprise disruption of microalgae by a method selected from pressure disruption, sonication, and ball milling

In additional embodiments, a method of the invention further comprises formulation of the homogenate, extract, or polysaccharides with a carrier suitable for human consumption. As described herein, the formulation may be that of tableting or encapsulation of the homogenate or extract.

In further embodiments, the methods comprise the use of microalgal homogenates, extracts, or polysaccharides wherein the cells contain an exogenous nucleic acid sequence, such as in the case of modified cells described herein. The exogenous sequence may encode a gene product capable of being expressed in the cells or be a sequence which increases expression of one or more endogenous microalgal gene product.

Non-limiting examples of the latter include insertion of regulator regions which increase expression of an endogenous microalgal gene and insertion of additional copies of an endogenous microalgal gene to increase copy number. Thus some embodiments of the invention include microalgal cells expressing an exogenous gene which increases production of a small molecule naturally produced by the microalgae or which induces the microalgae to produce, or directs the production of, a small molecule not naturally produced by the microalgae. In other embodiments, the increased expression of an endogenous microalgal gene or insertion of additional copies of an endogenous microalgal gene to increase copy number is used to increase production of a small molecule normally produced by the microalgae.

In yet further embodiments, the microalgal homogenates, extracts, or polysaccharides are from cells containing a modification to an endogenous nucleic acid sequence. One non-limiting example includes modified microalgal cells wherein an endogenous repressor nucleic acid sequence, or sequence encoding a proteinaceous or RNA gene product, is removed or inhibited such that production of a small molecule normally produced by the microalgae is increased.

Of course the invention includes embodiments wherein nucleic acid modification as described herein increases production of more than one microalgal small molecule.

In some embodiments, the small molecule of a microalgal cell which is increased by these methods of the invention is a carotenoid. Non-limiting examples of carotenoids include lycopene, lutein, beta carotene, zeaxanthin. In other embodiments, the small molecule is a polyunsaturated fatty acid, such as, but not limited to, EPA, DHA, linoleic acid and ARA.

In additional aspects, the invention includes a nutraceutical composition prepared by a method described herein. In some embodiments, the composition comprises homogenized red microalgal cells and a carrier suitable for human consumption. In other embodiments, the carrier is a food product or composition. The microalgal cells may be genetically modified as described above to result in red microalgae which produce an increased amount of a small molecule naturally produced by the red microalgae; or to produce a small molecule not naturally produced by the microalgae. In one non-limiting example, the small molecule is DHA.

The invention further provides for a combination composition wherein a microalgal homogenate further comprises an exopolysaccharide produced by the red microalgae. In some embodiments, the exopolysaccharide has been purified from culture media used to grow the red microalgae. The exopolysaccharide may be added to the cells before, during, or after homogenization. In another combination composition, a microalgal homogenate further comprises an exogenously added molecule, such as, but not limited to, EPA, DHA, linoleic acid, ARA, lycopene, lutein, beta carotene, and zeaxanthin.

A nutraceutical of the invention may also be a composition comprising a purified first polysaccharide produced from a microalgal species listed in Table 1 and a carrier suitable for human consumption. Non-limiting examples of the polysaccharides include sulfated molecules as well as polysaccharides with an average molecular weight (MW) of the polysaccharide is between about 2 and about 7 million Daltons (MDa). In some embodiments, the polysaccharide has an average MW of about 3, about 4.5, about 5, or about 6 MDa. In other embodiments, the average MW is below 2 MDa, such as below about 1, below about 0.8, below about 0.6, below about 0.4, or below about 0.2 MDa.

In some embodiments, the composition contains between 1 microgram and 50 grams of one type of microalgal polysaccharide. Alternatively, the composition contains more than one type of microalgal polysaccharide, such as one or more additional polysaccharide. In compositions with more than one type of polysaccharide, at least one polysaccharide is optionally from a non-microalgal source, such as a non-microalgal species. In some embodiments, the additional polysaccharide is beta glucan. In further embodiments, a composition further comprises a plant phytosterol.

In some aspects, a composition comprising both a microalgal homogenate and a polysaccharide, such as an exopolysaccharide, is disclosed herein. The composition may comprise homogenized microalgae and isolated or purified or semi-purified exopolysaccharide(s), wherein the composition is a percentage of exopolysaccharide by weight ranging from up to about 1% to up to about 20%, or higher. The remaining portion of the composition may be the homogenate or other carriers and excipients as desired for a composition, nutraceutical, or cosmeceutical of the invention. In some embodiments, the percentage of exopolysaccharide is up to about 2%, up to about 5%, or up to about 10%. This type of combination composition may be prepared by any appropriate means known to the skilled person, including preparing of each component separately and then combining them. In other methods, formulation of a composition comprises subjected a microalgal culture containing exopolysaccharides to tangential flow filtration to concentrate the material and then diafiltration until the composition is substantially free of salts, wherein the cells and exopolysaccharide are both retained in the retentate. The material can also be partially concentrated, diafiltered, and then concentrated further, and this regime can also be used on supernatant free of cells where the exopolysaccharide is retained. The exopolysaccharides may be those produced by the microalgae during culture or may be exogenously added to the culture before processing. The filtered material may then be homogenized or dried as described herein.

Other combination products are including in the invention. In some embodiments, a combination of a first composition for topical application and second composition for consumption is provided. In some embodiments, the first composition may be a topical formulation or non-systemic formulation, optionally a cosmeceutical, as described herein. Preferably, the first composition comprises a carrier suitable for topical application to skin, such as human skin. Non-limiting examples of the second composition include a food composition or nutraceutical as described herein. Preferably, the second composition comprises at least one carrier suitable for human consumption, such as that present in a food product or composition.

In some embodiments, the first and second compositions contain at least one compound in common. Non-limiting examples include one polysaccharide or one carrier in common. In other examples, the at least one compound is selected from DHA, EPA, ARA, lycopene, lutein, beta carotene, zeaxanthin, linoleic acid, vitamin C, and superoxide dismutase.

Combination products of the invention may be packaged separately for subsequent use together by a user or packaged together to facilitate purchase and use by a consumer. Packaging of the first and second compositions may be for sale as a single unit.

C. Methods of Use

A polysaccharide (as well as homogenate or extract) containing food product or nutraceutical of the invention may be consumed as a source of nutrition and/or sustenance. Thus the invention includes methods of providing food, nutrition or sustenance to a subject, such as a human being, by administration of a composition or nutraceutical as described herein. While a food product may be a primary source of sustenance, a nutraceutical may be used as a nutritional supplement. Thus the invention also includes methods of administering both to a subject. The administered food product may comprise a polysaccharide, extract, or homogenate as described herein. In some embodiments, the polysaccharide, extract or homogenate is used to thicken, stabilize or emulsify foods.

In other aspects, other methods for the use of a polysaccharide containing composition, including those containing a microalgal homogenate or extract of the invention, are disclosed. In some methods, the composition is used to regulate, or aid in the regulation of insulin. Administration of algal polysaccharides included in the invention reduces insulin secretion in response to a given stimulus. Subjects, including human beings, in need of insulin regulation may be identified by any means known to the skilled person. In some embodiments, the subject is identified as being at risk for diabetes by a skilled clinician. Being at risk includes having one or more risk factors, as assessed by the skilled person, which increase the chances of needing insulin regulation and/or having diabetes. Non-limiting examples of risk factors include those of lifestyle, behavior, health status, disease, and medication use. In some embodiments, the risk factors may amount to the present of “pre-diabetes” or “metabolic disease”.

Non-limiting examples of lifestyle factors include inactivity, stress, diet, and aging. Non-limiting examples of behavior factors include levels of sexual activity, smoking, alcohol use, and drug use. Non-limiting examples of health status factors include obesity, cholesterol, diabetes, immunosuppression, and hypertension as well as gender status as a woman, such as pregnancy, childbirth, and menopause. The compositions are particularly useful for lowering cholesterol levels in patients having abnormally high levels of cholesterol of at least 240 mg/dL total cholesterol, at least 160 mg/dL LDL cholesterol, no more than 40 mg/dL HDL cholesterol, and/or at least 400 mg/dL triglycerides.

Non-limiting examples of diseases include HIV, heart, cancer, and autoimmune diseases. Non-limiting examples of medications include use of contraceptives and steroids.

A nutraceutical of the invention may be administered to a subject found to have one or more of these risk factors sufficient to warrant conservative or aggressive treatment of the subject. The determination or diagnosis of risk factor presence may be conducted by a skilled person, such as a clinician. Non-limiting examples of conservative treatment methods may comprise administration of a polysaccharide composition of the invention optionally in combination of one or more alterations in activity to reduce one or more risk factors. Alternatively, the methods may be in the absence of other treatment for insulin malfunction or misregulation, pre-diabetes, or metabolic disease.

Non-limiting examples of aggressive treatment include active administration of a bioactive agent to a subject afflicted with diabetes or insulin misregulation or malfunction. Administration of a bioactive agent includes insulin injection to maintain glucose levels in a subject.

In some embodiments, a method of regulating insulin is provided. Such a method may comprise administering a polysaccharide produced by microalgae as described herein. The polysaccharides may reduce the need for other agents, such as a bioactive agent, that regulate insulin.

In further aspects, antioxidant properties of microalgal polysaccharides may be utilized to treat subjects in need of antioxidant activity. Polysaccharides with antioxidant activity may be identified by suitable means known to the skilled person. In some embodiments, the polysaccharides will be those from a Porphyridium species, such as one that has been subject to genetic and/or nutritional manipulation to produce polysaccharides with altered monosaccharide content and/or altered sulfation.

In some embodiments, antioxidant polysaccharides are used to inhibit, reduce or treat undesired inflammation. The inflammation can be the result of several diseases including autoimmune diseases, graft versus host disease, host versus graft disease, or pathogenic infections. In some embodiments, the polysaccharides will be those from a Porphyridium species, such as one that has been subject to genetic and/or nutritional manipulation to produce polysaccharides with altered monosaccharide content and/or altered sulfation.

Therefore, aspects of the invention include a method of reducing reactive oxygen species (ROS) in a mammal. In some embodiments, the method is used to prevent or treat a disease or unwanted condition associated with ROS or oxidative stress. Non-limiting examples of such a disease or unwanted condition include a neurodegenerative condition, atherosclerosis, metabolic syndrome, erectile dysfunction, transplant rejection, cancer, cardiovascular disease, hypertension, ischemia, reperfusion injury, rheumatoid arthritis, and aging, which are all conditions that are associated with increased in reactive oxygen species.

In some embodiments, a method comprises administering a composition comprising red microalgae cell material (such as a homogenate) as described herein. In other embodiments, the composition comprises one or more other agents or compounds with anti-oxidant activity. In further embodiments, the method may be used to lower the level of ROS, or reduce or decrease the amount of damage caused by ROS. The amount of the composition may be any that is effective or sufficient to produce a desired improvement or therapeutic benefit.

The method may be used to prevent, treat, or alleviate a symptom of, a neurodegenerative disease or disorder. Non-limiting examples of a neurodegenerative disease or disorder include dementias (e.g., senile dementia, memory disturbances/memory loss, dementias caused by neurodegenerative disorders (e.g., Alzheimer's, Parkinson's disease, Parkinson's disorders, Huntington's disease (Huntington's Chorea), Freidreich's ataxia, Lou Gehrig's disease, multiple sclerosis, Pick's disease, Parkinsonism dementia syndrome), progressive subcortical gliosis, progressive supranuclear palsy, thalmic degeneration syndrome, hereditary aphasia, amyotrophic lateral sclerosis, Shy-Drager syndrome, and Lewy body disease; vascular conditions (e.g., infarcts, hemorrhage, cardiac disorders); mixed vascular and Alzheimer's; bacterial meningitis; Creutzfeld-Jacob Disease; and Cushing's disease. In alternative embodiments, the method may be used to delay the onset of one or more of the above diseases or disorders in a mammal.

The method may also be used to prevent, treat, or alleviate metabolic syndrome in a human subject, which is characterized by at least three of the following: enlargement of the waist diameter, a higher level of arterial pressure, a higher level of low density lipoprotein cholesterol, a higher level of glycemia, and reduction of high density lipoprotein cholesterol (see for example, Palomo et al. Int. J. Mol. Med. (2006) 18(5):969-74).

The method may also be used to prevent, treat, or alleviate a symptom of, erectile dysfunction. The involvement of ROS in the disorder has been reported by Jeremy et al. (Int. J. Impot. Res. 2006 Oct. 19; Epub ahead of print).

The method may also be used to prevent, treat, or alleviate a symptom of, transplant rejection, such as in the case of organ or tissue transplant as non-limiting examples. The involvement of ROS in transplant rejection is known to the skilled person. See for example Cell Immunol. 2006 June; 241(2):59-65.

The method may also be used to prevent, treat, or alleviate a symptom of aging. The involvement of ROS in aging has been reported by Valko et al. (Int. J. Biochem Cell Biol. (2007) 39(1):44-84 Epub 2006 Aug. 4).

The invention includes a method to treat inflammation. Such a method may comprise administering a polysaccharide containing composition of the invention to a subject in need of anti-inflammatory activity. The polysaccharide may be one or more produced by microalgae described herein. The administering may be by a variety of means, including direct transfer to a tissue or subject via an intramuscular, intradermal, subdermal, subcutaneous, oral, parenteral, intraperitoneal, intrathecal, or intravenous procedure. Alternatively, a scaffold or binding protein can be placed within a cavity of the body, such as during surgery, or by inhalation, or vaginal or rectal administration.

In prophylactic applications, pharmaceutical compositions or medicaments are administered to a patient susceptible to, or otherwise at risk of, a disease or condition, such as excess cholesterol, inflammation, low insulin, inadequate joint lubrication in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease.

A microalgal cell material containing composition of the invention may also be used in an effective or sufficient amount to produce a desired improvement or therapeutic benefit in decreasing mammalian body fat (such as via a hypolipidemic effect), controlling or preventing weight gain in a mammal, reducing the body weight in a mammal, increasing satiety (or decreasing appetite) in a mammal, increasing energy expenditure (or energy use) in a mammal, reducing inflammation in a mammal, or preventing and/or treating atherosclerosis in a mammal.

The above applications of a composition may be embodied in a method comprising administering a composition comprising red microalgae cell material (such as a homogenate) for ingestion by a mammal as described herein. In some cases, the composition comprises one or more other agents or compounds with anti-oxidant activity.

### VII Gene Expression in Microalgae

Genes can be expressed in microalgae by providing, for example, coding sequences in operable linkage with promoters.

An exemplary vector design for expression of a gene in microalgae contains a first gene in operable linkage with a promoter active in algae, the first gene encoding a protein that imparts resistance to an antibiotic or herbicide. Optionally the first gene is followed by a 3′ untranslated sequence containing a polyadenylation signal. The vector may also contain a second promoter active in algae in operable linkage with a second gene. The second gene can encode any protein, for example an enzyme that produces small molecules or a mammalian growth hormone that can be advantageously present in a nutraceutical.

It is preferable to use codon-optimized cDNAs: for methods of recoding genes for expression in microalgae, see for example US patent application 20040209256.

It has been shown that many promoters in expression vectors are active in algae, including both promoters that are endogenous to the algae being transformed algae as well as promoters that are not endogenous to the algae being transformed (ie: promoters from other algae, promoters from plants, and promoters from plant viruses or algae viruses). Example of methods for transforming microalgae, in addition to those demonstrated in the Examples section below, including methods comprising the use of exogenous and/or endogenous promoters that are active in microalgae, and antibiotic resistance genes functional in microalgae, have been described. See for example; Curr Microbiol. 1997 December; 35(6):356-62 (Chlorella vulgaris); Mar Biotechnol (NY). 2002 January; 4(1):63-73 (Chlorella ellipsoidea); Mol Gen Genet. 1996 Oct. 16; 252(5):572-9 (Phaeodactylum tricornutum); Plant Mol. Biol. 1996 April; 31(1):1-12 (Volvox carteri); Proc Natl Acad Sci U S A. 1994 Nov. 22; 91(24):11562-6 (Volvox carteri); Falciatore A, Casotti R, Leblanc C, Abrescia C, Bowler C, PMID: 10383998, 1999 May; 1(3):239-251 (Laboratory of Molecular Plant Biology, Stazione Zoologica, VIIIa Comunale, I-80121 Naples, Italy) (Phaeodactylum tricornutum and Thalassiosira weissflogii); Plant Physiol. 2002 May; 129(1):7-12. (Porphyridium sp.); Proc Natl Acad Sci USA. 2003 Jan. 21; 100(2):438-42. (Chlamydomonas reinhardtii); Proc Natl Acad Sci USA. 1990 February; 87(3):1228-32. (Chlamydomonas reinhardtii); Nucleic Acids Res. 1992 Jun. 25; 20(12):2959-65; Mar Biotechnol (NY). 2002 January; 4(1):63-73 (Chlorella); Biochem Mol Biol Int. 1995 August; 36(5):1025-35 (Chlamydomonas reinhardtii); J. Microbiol. 2005 August; 43(4):361-5 (Dunaliella); Yi Chuan Xue Bao. 2005 April; 32(4):424-33 (Dunaliella); Mar Biotechnol (NY). 1999 May; 1(3):239-251. (Thalassiosira and Phaedactylum); Koksharova, Appl Microbiol Biotechnol 2002 February; 58(2):123-37 (various species); Mol Genet Genomics. 2004 February; 271(1):50-9 (Thermosynechococcus elongates); J. Bacteriol. (2000), 182, 211-215; FEMS Microbiol Lett. 2003 Apr. 25; 221(2):155-9; Plant Physiol. 1994 June; 105(2):635-41; Plant Mol. Biol. 1995 December; 29(5):897-907 (Synechococcus PCC 7942); Mar Pollut Bull. 2002; 45(1-12):163-7 (Anabaena PCC 7120); Proc Natl Acad Sci USA. 1984 March; 81(5):1561-5 (Anabaena (various strains)); Proc Natl Acad Sci USA. 2001 Mar. 27; 98(7):4243-8 (Synechocystis); Wirth, Mol Gen Genet. 1989 March; 216(1):175-7 (various species); Mol Microbiol, 2002 June; 44(6):1517-31 and Plasmid, 1993 September; 30(2):90-105 (Fremyella diplosiphon); Hall et al. (1993) Gene 124: 75-81 (Chlamydomonas reinhardtii); Gruber et al. (1991). Current Micro. 22: 15-20; Jarvis et al. (1991) Current Genet. 19: 317-322 (Chlorella); for additional promoters see also Table 1 from U.S. Pat. No. 6,027,900). For additional methods of transforming microalgae of the genus Porphyridium, see PCT application number WO2006013572, entitled “RED MICROALGAE EXPRESSING EXOGENOUS POLYPEPTIDES AND METHODS OF GENERATING AND UTILIZING SAME”, Application number WO2005IL00842 20050804, priority number(s) US20040598849P 20040805, which includes use of a cauliflower mosaic virus (CMV) promoter (See SEQ ID NO:45). Also see SEQ ID NO:44, a promoter that drives the gene encoding the glycoprotein that binds the polysaccharide from Porphyridium sp. (see GenBank accession number AY778963 for the cDNA of this gene). The promoter of SEQ ID NO:44 is a preferred promoter for driving exogenous genes in algae of the genus Porphyridium.

Suitable promoters may be used to express a nucleic acid sequence in microalgae. In some embodiments, the sequence is that of an exogenous gene or nucleic acid. In particular embodiments, the exogenous gene is one that encodes a carbohydrate transporter protein. Such a gene may be advantageously expressed in a microalgal cell to allow entry of a monosaccharide transported by the transporter protein. In other embodiments, the exogenous gene can encode a superoxide dismutase or a mammalian growth hormone. In cases of an exogenous nucleic acid coding sequence, the codon usage may be optionally optimized in whole or in part to facilitate expression in microalgae. In some embodiments the invention includes cells of the genus Porphyridium that have been stably transformed with a vector containing a selectable marker gene in operable linkage with a promoter active in microalgae. In other embodiments the invention includes cells of the genus Porphyridium that have been stably transformed with a vector containing a selectable marker gene in operable linkage with a promoter endogenous to a member of the Rhodophyte phylum. Such promoters include SEQ ID NOs: 44 and 55, promoters from the genome of Chondrus crispus (Genbank accession number Z47547), promoters from the genome of Cyanidioschyzon merolae (see for example Matsuzaki, M. et al. Nature 428, 653-657 (2004); Plant Physiology 137:567-585 (2005); entire sequence available at http://merolae.biol.s.u-tokyo.ac.ip/db/chromosome.cgi). In other embodiments the invention includes cells of the genus Porphyridium that have been stably transformed with a vector containing a selectable marker gene in operable linkage with a promoter other than a CMV promoter such as that found in PCT application WO2006013572.

The invention thus includes, in some embodiments, a microalgal cell comprising an exogenous gene that encodes a carbohydrate transporter protein. The cell may be that of the genus Porphyridium as a non-limiting example. Non-limiting examples of genes encoding carbohydrate transporters to facilitate the uptake of exogenously provided carbohydrates include genes encoding the proteins of SEQ ID NOs: 20, 22, 24, 26, 27, 29-39 and 46-48 as provided herein. In some embodiments the nucleic acid sequence encodes a protein with at least 60% amino acid sequence identity with a protein with a sequence represented by one of SEQ ID NOs: 20, 22, 24, 26, 27, and 29-39 and 46-48 wherein the protein is located in the plasma membrane of the cell and transports a carbohydrate from the culture media into the cell. In other embodiments, the nucleic acid sequence encodes a protein with at least about 70%, at least about 75%, at least about 80%, at least about 85%, at least about 90%, at least about 95%, or at least about 98%, or higher, amino acid identity with a sequence of these SEQ ID NOs: 20, 22, 24, 26 and 27. In further embodiments, the nucleic acid sequence has at least 60% nucleotide identity with a nucleic acid molecule with a one of SEQ ID NOs: 21, 23 and 25. In other embodiments, the nucleic acid sequence has at least about 70%, at least about 75%, at least about 80%, at least about 85%, at least about 90%, at least about 95%, or at least about 98%, or higher, nucleic acid identity with a sequence of these SEQ ID NOs.

Hexokinases can also be expressed to facilitate utilization of exogenously provided carbohydrates through phosphorylation of monosaccharides. A nonlimiting example of a hexokinase is SEQ IS NO: 71 or a protein with at least 60% amino acid identity with SEQ ID NO:71 that phosphorylates a monosaccharide.

In other embodiments, the invention provides for the expression of a protein sequence found to be tightly associated with microalgal polysaccharides. One non-limiting example is the protein of SEQ ID NO: 28, which has been shown to be tightly associated with, but not covalently bound to, the polysaccharide from Porphyridium sp. (see J. Phycol. 40: 568-580 (2004)). When Porphyridium culture media is subjected to tangential flow filtration using a filter containing a pore size well in excess of the molecular weight of the protein of SEQ ID NO: 28, the polysaccharide in the retentate contains detectable amounts of the protein, indicating its tight association with the polysaccharide. The calculated molecular weight of the protein is approximately 58 kD, however with glycosylation the protein is approximately 66 kD.

Such a protein may be expressed directly such that it will be present with the polysaccharides of the invention or expressed as part of a fusion or chimeric protein as described herein. As a fusion protein, the portion that is tightly associated with a microalgal polysaccharide effectively links the other portion(s) to the polysaccharide. A fusion protein may comprise a second protein or polypeptide, with a homogenous or heterologous sequence. A homogenous sequence would result in a dimer or multimer of the protein while a heterologous sequence can introduce a new functionality, including that of a bioactive protein or polypeptide.

Non-limiting examples of the second protein include an antibody, a growth hormone or factor, and an enzyme. In optional embodiments, the enzyme is superoxide dismutase, such as that has at least about 60% amino acid identity with the sequence of SEQ ID NO: 14 or SEQ ID NO: 15 as non-limiting examples. Superoxide dismutase scavenges reactive oxygen species such as the superoxide anion. In some embodiments, the superoxide dismutase has at least about 70%, at least about 75%, at least about 80%, at least about 85%, at least about 90%, at least about 95%, or at least about 98%, or higher, amino acid identity with the sequence of SEQ ID NO:14 or 15 or a sequence from Table 16. In other embodiments, the enzyme is a phytase (such as GenBank accession number CAB91845 and U.S. Pat. Nos. 6,855,365 and 6,110,719).

A fusion between the polysaccharide binding protein and antibodies that specifically bind to and neutralize a pathogen are included in the invention. Non-limiting examples include anti-HIV antibodies, like the 2G12 antibody (see Proc Natl Acad Sci USA. 2005 Sep. 20; 102(38):13372-7); the 1RHH_B antibody (see Clin Exp Immunol. 2005 July; 141(1):72-80); the scFv102 antibody (see J Gen Virol. 2005 June; 86(Pt 6):1791-800); and the microAb antibody (see Nat. Med. 2005 June; 11(6):615-22; 2G12, 2F5, 4E10, 2g12 Fab 1ZLS_L). These and other antibodies, preferably antibodies that specifically bind to infectious disease agents, can also be expressed in algae without being fused to any other proteins. The biomass containing the recombinant antibodies can be administered orally to deliver the antibodies to a mammal for prophylaxis or treatment.

One advantage to a fusion is that the bioactivity of the polysaccharide and the bioactivity from the protein can be combined in a product without increasing the manufacturing cost over only purifying the polysaccharide. As a non-limiting example, the potent antioxidant properties of a Porphyridium polysaccharide can be combined with the potent antioxidant properties of superoxide dismutase in a fusion, however the polysaccharide:superoxide dismutase combination can be isolated to a high level of purity using tangential flow filtration. In another non-limiting example, the potent antiviral properties of a Porphyridium polysaccharide can be added to the potent neutralizing activity of recombinant antibodies fused to the protein (SEQ ID NO:28) that tightly associates with the polysaccharide. In a preferred embodiment, fusion proteins of SEQ ID NO:28 or sequences with at least 60% amino acid identity with SEQ ID NO:28 bind with high affinity to a sulfated exopolysaccharide from a cell of the genus Porphyridium. It is preferred but not becessary that the binding be selective.

In other embodiments, the exogenous gene can encode a protein that catalyzes the conversion of one carotenoids/xanthophyll to another, such as a beta-carotene hydroxylase (beta carotene to zeaxanthin), a lycopene cyclase (lycopene to beta carotene), and other enzymes known in the art. In cases of an exogenous nucleic acid coding sequence, the codon usage may be optionally optimized in whole or in part to facilitate expression in microalgae. Beta carotene hydroxylases exhibit a wide degree of amino acid identity despite the fact that that are capable of catalyzing the same reaction. The invention includes cloning and expression of enzymes that catalyze the conversion of beta carotene to zeaxanthin that have at least 25% amino acid identity to those in Table 4. Optionally, the beta carotene hydroxylases of the invention have at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, and at least 99% amino acid identity to those in Table 4.

The invention includes cloning and expression of enzymes that catalyze the conversion of lycopene to beta carotene (ie: lycopene cylases). Optionally, the lycopene cylases of the invention have at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, and at least 99% amino acid identity to GenBank accession numbers AAA81880, NP_{—}624526, BAA20275, and P21687.

Beta carotene hydroxylases exhibit widely divergent amino acid identity while exhibiting the same catalytic activity. For example, BLAST comparisons of beta carotene hydroxylases show as little as 25% amino acid identity: NP_{—}682690 vs. NP_{—}896386 (59% identity); ABB49299 vs. YP_{—}172377 (67% identity); NP_{—}682690 vs. AAT38625 (25% identity); YP_{—}457405 vs. NP_{—}440788 (25% identity); ZP_{—}01084736 vs. NP_{—}848964 (55% identity). BLAST parameters used in the analysis were default parameters (blastp program, matrix BLOSUM62, open gap 11 and extension gap 1, gapx_dropoff 50, expect 10.0, word size 3, filter activated).

In other embodiments, the invention includes genetic expression methods comprising the use of an expression vector. In one method, a microalgal cell, such as a Porphyridium cell, is transformed with a dual expression vector under conditions wherein vector mediated gene expression occurs. The expression vector may comprise a resistance cassette comprising a gene encoding a protein that confers resistance to an antibiotic, such as zeocin, or another selectable marker such as a carbohydrate transporter gene for selection in the dark in the presence of a fixed carbon source, operably linked to a promoter active in microalgae. The vector may also comprise a second expression cassette comprising a second protein to a promoter active in microalgae. The two cassettes are physically linked in the vector. The transformed cells may be optionally selected based upon the ability to grow in the presence of the antibiotic or other selectable marker under conditions wherein cells lacking the resistance cassette would not grow, such as in the dark. The resistance cassette, as well as the expression cassette, may be taken in whole or in part from another vector molecule.

In one non-limiting example, a method of expressing an exogenous gene in a cell of the genus Porphyridium is provided. The method may comprise operably linking a gene encoding a protein that confers resistance to the antibiotic zeocin to a promoter active in microalgae to form a resistance cassette; operably linking a gene encoding a second protein to a promoter active in microalgae to form a second expression cassette, wherein the resistance cassette and second expression cassette are physically connected to form a dual expression vector; transforming the cell with the dual expression vector; and selecting for the ability to survive in the presence of at least 2.5 ug/ml zeocin, preferably at least 3.0 ug/ml zeocin, and more preferably at least 3.5 ug/ml zeocin, more preferably at least 5.0 ug/ml zeocin.

In some embodiments, the expression cassette expresses a growth hormone, optionally mammalian and optionally a fish or other vertebrate growth hormone. Non-limiting examples of the growth hormone include bovine growth hormone, human growth hormone, porcine growth hormone, and equine growth hormone. Other non-limiting examples growth hormones include proteins with sequences represented as SEQ ID NOs: 17, 18 and 19. In other embodiments, the growth hormone has at least about 60%, at least about 70%, at least about 75%, at least about 80%, at least about 85%, at least about 90%, at least about 95%, or at least about 98%, or higher, amino acid identity with a protein sequence represented by one of SEQ ID NOs:17, 18, 19 or one of Genbank accession numbers AAW67479; AAW67480; AAB64117; BAC07248; AAF61751; AAX77220; ABA55647; ABI97975; BAA06379; AAU93606; AAX31661; NP_{—}001003168, further wherein the protein stimulates growth in a vertebrate organism.

In another embodiment the expression cassette expresses a phytase enzyme, such as for example, SEQ ID NOs: 40 and 41.

In other aspects, the invention provides the cells, such as Porphyridium cells, prepared by the above methods. The cells, whether in whole or extracted or homogenized form, may comprise a mammalian growth hormone via recombinant protein expression as provided above. In a preferred embodiment, transgenic Porphyridium expressing a mammalian growth hormone and/or a phytase enzyme are formulated into livestock food and administered to animals.

The cells may be advantageously used as, or as a component of, animal feed. The advantage to expressing such growth hormones in microalgae such as Porphyridium is that the polysaccharide is not hydrolyzed by the stomach of the mammal (see for example Br J. Nutr. 2000 October; 84(4):469-76), and therefore the cell wall, which is made primarily of polysaccharide, protects the mammalian growth hormone as the cells transit through the stomach and into the intestines. Once in the intestines, the cell wall eventually begins breaking down, allowing the growth hormones to cross into the bloodstream and achieve a pharmacological effect.

In additional aspects, the expression of a protein that produces small molecules in microalgae is included and described. Some genes that can be expressed using the methods provided herein encode enzymes that produce nutraceutical small molecules such as lutein, zeaxanthin, and DHA. Preferably the genes encoding the proteins are synthetic and are created using preferred codons on the microalgae in which the gene is to be expressed. For example, enzyme capable of turning EPA into DHA are cloned into the microalgae Porphyridium sp. by recoding genes to adapt to Porphyridium sp. preferred codons. For examples of such enzymes see Nat. Biotechnol. 2005 August; 23(8):1013-7. For examples of enzymes in the carotenoid pathway see SEQ ID NOs: 12 and 13 and sequences from Table 15. The advantage to expressing such genes is that the nutraceutical value of the cells increases without increasing the manufacturing cost of producing the cells.

For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### VIII Methods of Trophic Conversion

As explained herein, microalgae generally have the ability to live off a fixed carbon sources such as glucose, but many do not have transporters that allow for uptake of the fixed carbon source from the culture media. Microalgae cells can be transformed with a gene that encodes a plasma membrane sugar transporter that allows for the selection of growth in the dark, in the absence of photosynthesis, in the presence of the transporter's substrate sugar. Such transformed cells provide a significant benefit in that the need for light energy is reduced or eliminated because the cells may grow and produce cellular products, including polysaccharides, in the presence of fixed carbon material as the energy source. See for example, Science. 2001 Jun. 15; 292(5524):2073-5. Such growth achieves much higher cell densities in a shorter period of time than photoautotrophic growth.

The transformed microalgal cell may be one that is described above as expressing a sugar transporter. Nucleic acids and vectors for such expression are also described above. For example, nucleic acids encoding carbohydrate transporters such as SEQ ID NOs: 20, 22, 24, 26, 27, 29-39 and 46-48 are placed in operable linkage with a promoter active in microalgae. Preferably, the nucleic acid encoding a carbohydrate transporter contains preferred codons of the organism the vector is transformed into. For example, the nucleic acids of SEQ ID NOs: 21, 23, and 25 encode the carbohydrate transporter proteins of SEQ ID NOs: 20, 22, and 24, respectively. As a nonlimiting example, a codon-optimized cDNA encoding a carbohydrate transporter protein, optimized for expression in Porphyridium sp., is placed in operable linkage with a promoter and 3′UTR active in microalgae. The vector is used to transform a cell of the genus Porphyridium using methods disclosed herein, including biolistic transformation, electroporation, and glass bead transformation. A preferred promoter is active in more than one species of microalgae, such as for example the Chlamydomonas reinhardtii RBCS2 promoter (SEQ ID NO:43). Any promoter active in microalgae can be used to express a gene in such constructs, and preferred promoters such as RBCS2 and viral promoters have been shown to be active in multiple species of microalgae (see for example Plant Cell Rep. 2005 March; 23(10-11):727-35; J. Microbiol. 2005 August; 43(4):361-5; Mar Biotechnol (NY). 2002 January; 4(1):63-73). Promoters, cDNAs, and 3′UTRs, as well as other elements of the vectors, can be generated through cloning techniques using fragments isolated from native sources (see for example Molecular Cloning: A Laboratory Manual, Sambrook et al. (3d edition, 2001, Cold Spring Harbor Press; and U.S. Pat. No. 4,683,202). Alternatively, elements can be generated synthetically using known methods (see for example Gene. 1995 Oct. 16; 164(1):49-53).

Alternatively, cells may be mutagenized and then selected for the ability to grow in the absence of light energy but in the presence of a fixed carbon source.

Thus the invention includes a method of producing microalgal cells that have gained the ability to grow via a fixed carbon source in the absence of photosynthesis. This may also be referred to as trophic conversion of a microalgal cell to no longer be an obligate photoautotroph. In some embodiments, the method comprises identifying or selecting cells that have gained the ability to utilize energy from a fixed carbon source.

In some embodiments, the methods comprise selecting microalgal cells, such as a Porphyridium cell, for the ability to undergo cell division in the absence of light, or light energy. The cells, such as one from a species listed in Table 1, may be those which have been transformed with a sugar transporter or those which have been mutagenized, chemically or non-chemically. The selection may be, for example, on about 0.1% or about 1% glucose, or another fixed carbon source, in the dark. Preferred fixed carbon compounds are listed in Tables 2 and 3.

Non-limiting examples of carbohydrate transporter proteins, optionally operably linked to promoters active in microalgae, as well as expression cassettes and vectors comprising them, have been described above. Alternatively, the nucleic acids may be incorporated into the genome of a microalgal cell such that an endogenous promoter is used to express the transporter. Additional embodiments of the methods include expression of transporters of a carbohydrate selected from Table 2 or 3. Non-limiting examples of mutagenesis include contact or propagation in the presence of a mutagen, such as ultraviolet light, nitrosoguanidine, and/or ethane methyl sulfonate (EMS).

As one non-limiting example, a method of the invention comprises providing a nucleic acid encoding a carbohydrate transporter protein; transforming a Porphyridium cell with the nucleic acid; and selecting for the ability to undergo cell division in the absence of light or in the presence of a carbohydrate that is transported by the carbohydrate transporter protein. In another non-limiting example, a method comprises subjecting a microalgal cell to a mutagen; placing the cell in the presence of a molecule listed in Tables 2 or 3; and selecting for the ability to undergo cell division in the absence of light.

The methods may also be considered to be for trophically converting a microalgal cell to no longer be an obligate phototroph. It is pointed out that the ability to select for loss of obligate phototrophism also provides an alternative means to select for expression of a sugar transporter in the absence of a selectable marker because correct expression and functionality of the transporter is the selectable phenotype when cells are grown in the absence of light for photosynthesis.

It should be apparent to one skilled in the art that various embodiments and modifications may be made to the invention disclosed in this application without departing from the scope and spirit of the invention. All publications mentioned herein are cited for the purpose of describing and disclosing reagents, methodologies and concepts that may be used in connection with the present invention. Nothing herein is to be construed as an admission that these references are prior art in relation to the inventions described herein.

### Example 1

### Growth of Porphyridium cruentum and Porphyridium sp.

Porphyridium sp. (strain UTEX 637) and Porphyridium cruentum (strain UTEX 161) were inoculated into autoclaved 2 liter Erlenmeyer flasks containing an artificial seawater media: 1495 ASW medium recipe from the American Type Culture Collection, (components are per 1 liter of media) NaCl: 27.0 g; MgSO₄. 7H₂O: 6.6 g; MgCl₂. 6H₂O: 5.6 g; CaCl₂. 2H₂O: 1.5 g; KNO₃: 1.0 g; KH₂PO₄: 0.07 g; NaHCO₃: 0.04 g; 1.0 M Tris-HCl buffer, pH 7.6:20.0 ml; Trace Metal Solution (see below): 1.0 ml; Chelated Iron Solution (see below): 1.0 ml; Distilled water: bring to 1.0 L. Trace Metal Solution: ZnCl₂:4.0 mg; H₃BO₃: 60.0 mg; CoCl₂. 6H₂O: 1.5 mg; CuCl₂.2H₂O: 4.0 mg; MnCl₂. 4H₂O: 40.0 mg; (NH₄)₆Mo₇O₂₄. 4H₂O: 37.0 mg; Distilled water: 100.0 ml. Chelated Iron Solution: FeCl₃. 4H₂O: 240.0 mg, 0.05 M EDTA, pH 7.6: 100.0 ml. Media was autoclaved for at least 15 minutes at 121° C.

Inoculated cultures in 2 liter flasks were maintained at room temperature on stir plates. Stir bars were placed in the flasks before autoclaving. A mixture of 5% CO₂ and air was bubbled into the flasks. Gas was filter sterilized before entry. The flasks were under 24 hour illumination from above by standard fluorescent lights (approximately 150 uE/m⁻¹/s⁻¹). Cells were grown for approximately 12 days, at which point the cultures contained approximately of 4×10⁶ cells/mL.

### Example 2

Dense Porphyridium sp. and Porphyridium cruentum cultures were centrifuged at 4000 rcf. The supernatant was subjected to tangential flow filtration in a Millipore Pellicon 2 device through a 1000 kD regenerated cellulose membrane (filter catalog number P2C01MC01). Approximately 4.1 liters of Porphyridium cruentum and 15 liters of Porphyridium sp. supernatants were concentrated to a volume of approximately 200 ml in separate experiments. The concentrated exopolysaccharide solutions were then diafiltered with 10 liters of 1 mM Tris (pH 7.5). The retentate was then flushed with 1 mM Tris (pH 7.5), and the total recovered polysaccharide was lyophilized to completion. Yield calculations were performed by the dimethylmethylene blue (DMMB) assay. The lyophilized polysaccharide was resuspended in deionized water and protein was measured by the bicinchoninic acid (BCA) method. Total dry product measured after lyophilization was 3.28 g for Porphyridium sp. and 2.0 g for Porphyridium cruentum. Total protein calculated as a percentage of total dry product was 12.6% for Porphyridium sp. and 15.0% for Porphyridium cruentum.

### Example 3

A measured mass (approximately 125 grams) of freshly harvested Porphyridium sp. cells, resuspended in a minimum amount of dH₂O sufficient to allow the cells to flow as a liquid, was placed in a container. The cells were subjected to increasing amounts of sonication over time at a predetermined sonication level. Samples were drawn at predetermined time intervals, suspended in measured volume of dH₂O and diluted appropriately to allow visual observation under a microscope and measurement of polysaccharide concentration of the cell suspension using the DMMB assay. A plot was made of the total amount of time for which the biomass was sonicated and the polysaccharide concentration of the biomass suspension. Two experiments were conducted with different time intervals and total time the sample was subjected to sonication. The first data set from sonication experiment 1 was obtained by subjecting the sample to sonication for a total time period of 60 minutes in 5 minute increments. The second data set from sonication experiment 2 was obtained by subjecting the sample to sonication for a total time period of 6 minutes in 1-minute increments. The data, observations and experimental details are described below. Standard curves were generated using TFF-purified, lyophilized, weighed, resuspended Porphyridium sp. exopolysaccharide.

General Parameters of Sonication Experiments 1 and 2

Cells were collected and volume of the culture was measured. The biomass was separated from the culture solution by centrifugation. The centrifuge used was a Form a Scientific Centra-GP8R refrigerated centrifuge. The parameters used for centrifugation were 4200 rpm, 8 minutes, rotor#218. Following centrifugation, the biomass was washed with dH₂O. The supernatant from the washings was discarded and the pelleted cell biomass was collected for the experiment.

A sample of 100 μL of the biomass suspension was collected at time point 0 (0TP) and suspended in 900 μL dH₂O. The suspension was further diluted ten-fold and used for visual observation and DMMB assay. The time point 0 sample represents the solvent-available polysaccharide concentration in the cell suspension before the cells were subjected to sonication. This was the baseline polysaccharide value for the experiments.

The following sonication parameters were set: power level=8, 20 seconds ON/20 seconds OFF (Misonix 3000 Sonicator with flat probe tip). The container with the biomass was placed in an ice bath to prevent overheating and the ice was replenished as necessary. The sample was prepared as follows for visual observation and DMMB assay: 100 μL of the biomass sample+900 μL dH₂O was labeled as dilution 1. 100 μL of (i) dilution 1+900 μL dH₂O for cell observation and DMMB assay.

Sonication Experiment 1

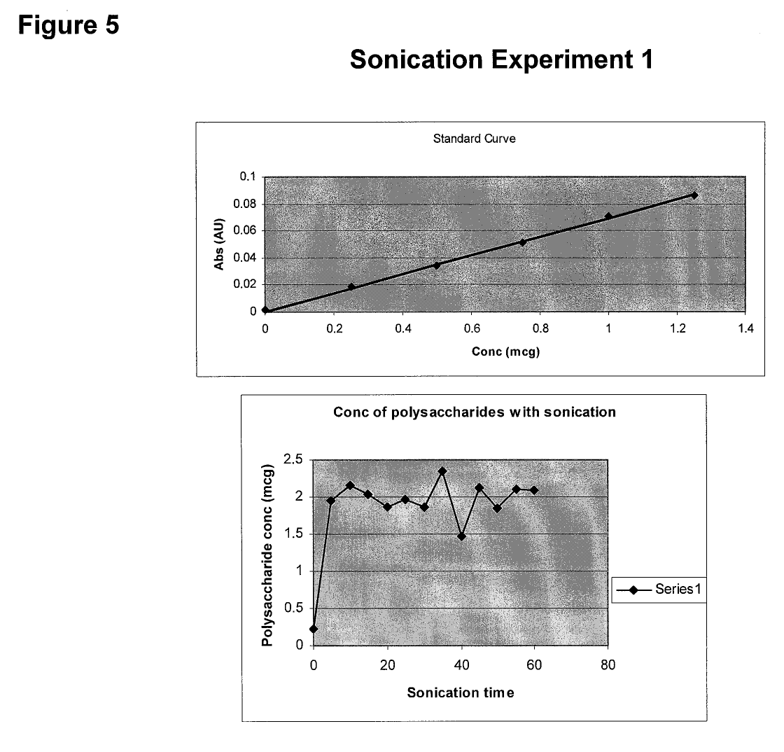

In the first experiment the sample was sonicated for a total time period of 60 minutes, in 5-minute increments (20 seconds ON/20 seconds OFF). The data is presented in Tables 4, 5 and 6. The plots of the absorbance results are presented in FIG. 5.

TABLE 4
SONICATION RECORD - EXPERIMENT 1

| | Time point | |
|---|---|---|
| Ser # | (min) | Observations |
| 1 | 0 | Healthy red cells |
| 2 | 5 | Red color disappeared, small greenish circular |
| | | particles |
| 3 | 10 | Small particle, smaller than 5 minute TP |
| 4 | 15 | Small particle, smaller than 10 minute TP. Same |
| | | observation as 10 minute time |
| 5 | 20 | Similar to 15 minute TP. Small particles; empty |
| | | circular shells in the field of vision |
| 6 | 25 | Similar to 20 minute TP |
| 7 | 30 | Similar to 25 minute TP, particles less numerous |
| 8 | 35 | Similar to 30 minute TP |
| 9 | 40 | Similar to 35 minute TP |
| 10 | 45 | Similar to 40 minute TP |
| 11 | 50 | Very few shells, mostly fine particles |
| 12 | 55 | Similar to 50 minute TP. |
| 13 | 60 | Fine particles, hardly any shells |

TP = time point.

TABLE 5
STANDARD CURVE RECORD - SONICATION EXPERIMENT 1

| Absorbance (AU) | Concentration (μg) |
|---|---|
| 0 | Blank, 0 |
| 0.02 | 0.25 |
| 0.03 | 0.5 |
| 0.05 | 0.75 |
| 0.07 | 1.0 |
| 0.09 | 1.25 |

TABLE 6
Record of Sample Absorbance versus Time Points - Sonication
Experiment 1

| SAMPLE | Solvent-Available |
|---|---|
| TIME POINT | Polysaccharide |
| (MIN) | (μg) |
| 0 | 0.23 |
| 5 | 1.95 |
| 10 | 2.16 |
| 15 | 2.03 |
| 20 | 1.86 |
| 25 | 1.97 |
| 30 | 1.87 |
| 35 | 2.35 |
| 40 | 1.47 |
| 45 | 2.12 |
| 50 | 1.84 |
| 55 | 2.1 |
| 60 | 2.09 |

The plot of polysaccharide concentration versus sonication time points is displayed above and in FIG. 5. Solvent-available polysaccharide concentration of the biomass (cell) suspension reaches a maximum value after 5 minutes of sonication. Additional sonication in 5-minute increments did not result in increased solvent-available polysaccharide concentration.

Homogenization by sonication of the biomass resulted in an approximately 10-fold increase in solvent-available polysaccharide concentration of the biomass suspension, indicating that homogenization significantly enhances the amount of polysaccharide available to the solvent. These results demonstrate that physically disrupted compositions of Porphyridium for oral or other administration provide novel and unexpected levels or polysaccharide bioavailability compared to compositions of intact cells. Visual observation of the samples also indicates rupture of the cell wall and thus release of insoluble cell wall-bound polysaccharides from the cells into the solution that is measured as the increased polysaccharide concentration in the biomass suspension.

### Sonication Experiment 2

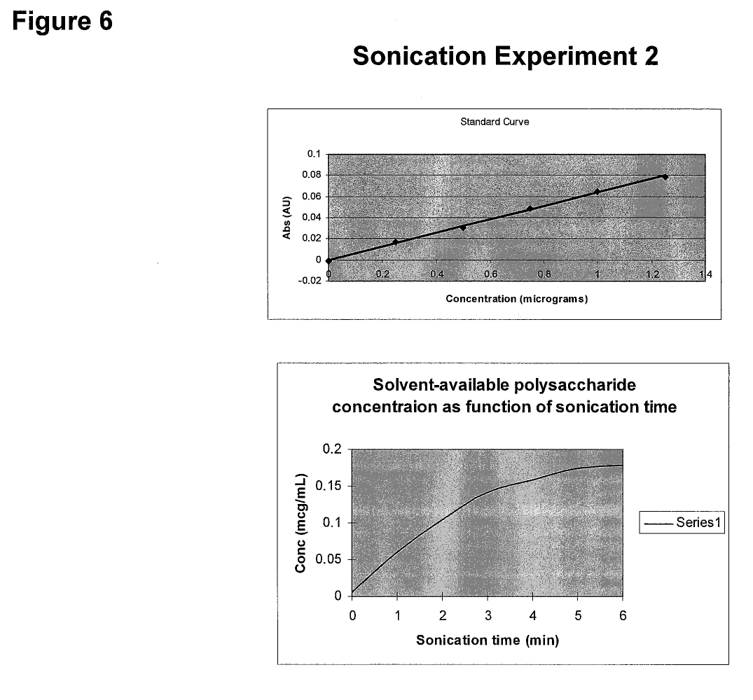

In the second experiment the sample was sonicated for a total time period of 6 minutes in 1-minute increments. The data is presented in Tables 7, 8 and 9. The plots of the absorbance results are presented in FIG. 6.

TABLE 7
SONICATION EXPERIMENT 2

| | Time | |
|---|---|---|
| | point | |
| Ser # | (min) | Observations |
| 1 | 0 | Healthy red-brown cells appear circular |
| 2 | 1 | Circular particles scattered in the field of vision with few |
| | | healthy cells. Red color has mostly disappeared from cell |
| | | bodies. |
| 3 | 2 | Observation similar to time point 2 minute. |
| 4 | 3 | Very few healthy cells present. Red color has disappeared |
| | | and the concentration of particles closer in size to whole |
| | | cells has decreased dramatically. |
| 5 | 4 | Whole cells are completely absent. The particles are |
| | | smaller and fewer in number. |
| 6 | 5 | Observation similar to time point 5 minute. |
| 7 | 6 | Whole cells are completely absent. Large particles are |
| | | completely absent. |

TABLE 8
STANDARD CURVE RECORD - SONICATION EXPERIMENT 2

| Absorbance (AU) | Concentration (μg) |
|---|---|
| −0.001 | Blank, 0 |
| 0.017 | 0.25 |
| 0.031 | 0.5 |
| 0.049 | 0.75 |
| 0.0645 | 1.0 |
| 0.079 | 1.25 |

TABLE 9
Record of Sample Absorbance versus Time Points - Sonication
Experiment 2

| SAMPLE | Solvent-Available |
|---|---|
| TIME POINT | Polysaccharide |
| (MIN) | (μg) |
| 0 | 0.063 |
| 1 | 0.6 |
| 2 | 1.04 |
| 3 | 1.41 |
| 4 | 1.59 |
| 5 | 1.74 |
| 6 | 1.78 |

The value of the solvent-available polysaccharide increases gradually up to the 5 minute time point as shown in Table 9 and FIG. 6.

### Example 4

Porphyridium sp. culture was centrifuged at 4000 rcf and supernatant was collected. The supernatant was divided into six 30 ml aliquots. Three aliquots were autoclaved for 15 min at 121° C. After cooling to room temperature, one aliquot was mixed with methanol (58.3% vol/vol), one was mixed with ethanol (47.5% vol/vol) and one was mixed with isopropanol (50% vol/vol). The same concentrations of these alcohols were added to the three supernatant aliquots that were not autoclaved. Polysaccharide precipitates from all six samples were collected immediately by centrifugation at 4000 rcf at 20° C. for 10 min and pellets were washed in 20% of their respective alcohols. Pellets were then dried by lyophilization and resuspended in 15 ml deionized water by placement in a 60° C. water bath. Polysaccharide pellets from non-autoclaved samples were partially soluble or insoluble. Polysaccharide pellets from autoclaved ethanol and methanol precipitation were partially soluble. The polysaccharide pellet obtained from isopropanol precipitation of the autoclaved supernatant was completely soluble in water.

### Example 5

Approximately 10 milligrams of purified polysaccharide from Porphyridium sp. and Porphyridium cruentum (described in Example 3) were subjected to monosaccharide analysis.

Monosaccharide analysis was performed by combined gas chromatography/mass spectrometry (GC/MS) of the per-O-trimethylsilyl (TMS) derivatives of the monosaccharide methyl glycosides produced from the sample by acidic methanolysis.

Methyl glycosides prepared from 500 μg of the dry sample provided by the client by methanolysis in 1 M HCl in methanol at 80° C. (18-22 hours), followed by re-N-acetylation with pyridine and acetic anhydride in methanol (for detection of amino sugars). The samples were then per-O-trimethylsilylated by treatment with Tri-Sil (Pierce) at 80° C. (30 mins). These procedures were carried out as previously described in Merkle and Poppe (1994) Methods Enzymol. 230:1-15; York, et al. (1985) Methods Enzymol. 118:3-40. GC/MS analysis of the TMS methyl glycosides was performed on an HP 5890 GC interfaced to a 5970 MSD, using a Supelco DB-1 fused silica capillary column (30 m 0.25 mm ID).

TABLE 10
Porphyridium sp. monosaccharide analysis

| Glycosyl residue | Mass (μg) | Mole % |
|---|---|---|
| Arabinose (Ara) | n.d. | n.d. |
| Rhamnose (Rha) | 2.7 | 1.6 |
| Fucose (Fuc) | n.d. | n.d. |
| Xylose (Xyl) | 70.2 | 44.2 |
| Glucuronic acid (GlcA) | n.d. | n.d. |
| Galacturonic acid (GalA) | n.d. | n.d. |
| Mannose (Man) | 3.5 | 1.8 |
| Galactose (Gal) | 65.4 | 34.2 |
| Glucose (Glc) | 34.7 | 18.2 |
| N-acetyl galactosamine (GalNAc) | n.d. | n.d. |
| N-acetyl glucosamine (GlcNAc) | trace | trace |
| | Σ = 176.5 | |

TABLE 11
Porphyridium cruentum monosaccharide analysis

| Glycosyl residue | Mass (μg) | Mole % |
|---|---|---|
| Arabinose (Ara) | n.d. | n.d. |
| Rhamnose (Rha) | n.d. | n.d. |
| Fucose (Fuc) | n.d. | n.d. |
| Xylose (Xyl) | 148.8 | 53.2 |
| Glucuronic Acid (GlcA) | 14.8 | 4.1 |
| Mannose (Man) | n.d. | n.d. |
| Galactose (Gal) | 88.3 | 26.3 |
| Glucose (Glc) | 55.0 | 16.4 |
| N-acetyl glucosamine (GlcNAc) | trace | trace |
| N-acetyl neuraminic acid (NANA) | n.d. | n.d. |
| | Σ = 292.1 | |

Mole % values are expressed as mole percent of total carbohydrate in the sample.
n.d. = none detected.

### Example 6

Porphyridium sp. was grown as described. 2 liters of centrifuged Porphyridium sp. culture supernatant were autoclaved at 121° C. for 20 minutes and then treated with 50% isopropanol to precipitate polysaccharides. Prior to autoclaving the 2 liters of supernatant contained 90.38 mg polysaccharide. The pellet was washed with 20% isopropanol and dried by lyophilization. The dried material was resuspended in deionized water. The resuspended polysaccharide solution was dialyzed to completion against deionized water in a Spectra/Por cellulose ester dialysis membrane (25,000 MWCO). 4.24% of the solid content in the solution was proteins as measured by the BCA assay.

### Example 7

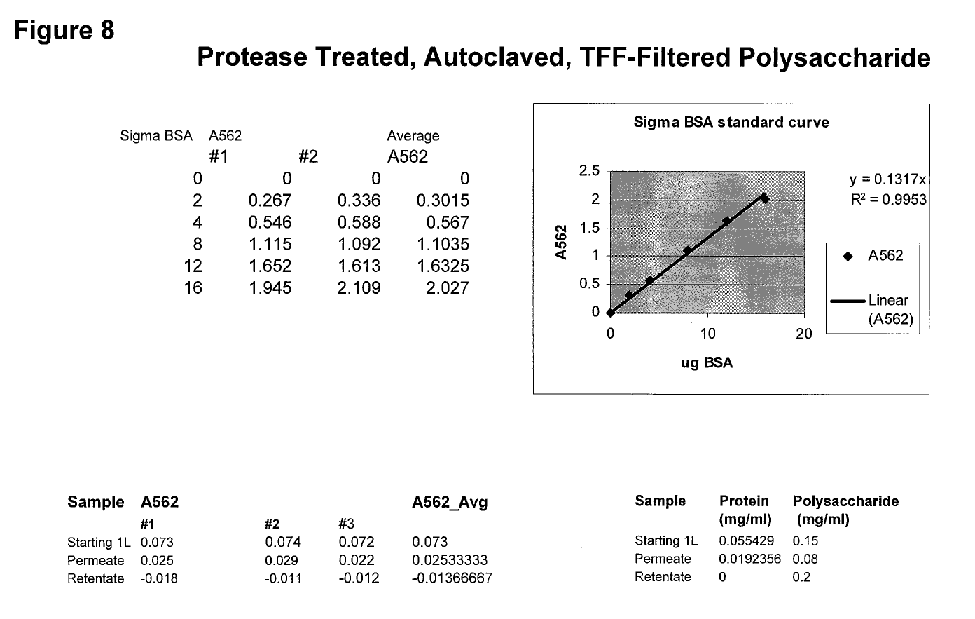

Porphyridium sp. was grown as described. 1 liters of centrifuged Porphyridium sp. culture supernatant was autoclaved at 121° C. for 15 minutes and then treated with 10% protease (Sigma catalog number P-5147; protease treatment amount relative to protein content of the supernatant as determined by BCA assay). The protease reaction proceeded for 4 days at 37° C. The solution was then subjected to tangential flow filtration in a Millipore Pellicon® cassette system using a 0.1 micrometer regenerated cellulose membrane. The retentate was diafiltered to completion with deionized water. No protein was detected in the diafiltered retentate by the BCA assay. See FIG. 8.

Optionally, the retentate can be autoclaved to achieve sterility if the filtration system is not sterile. Optionally the sterile retentate can be mixed with pharmaceutically acceptable carrier(s) and filled in vials for injection.

Optionally, the protein free polysaccharide can be fragmented by, for example, sonication to reduce viscosity for parenteral injection as, for example, an antiviral compound. Preferably the sterile polysaccharide is not fragmented when prepared for injection as a joint lubricant.

### Example 8

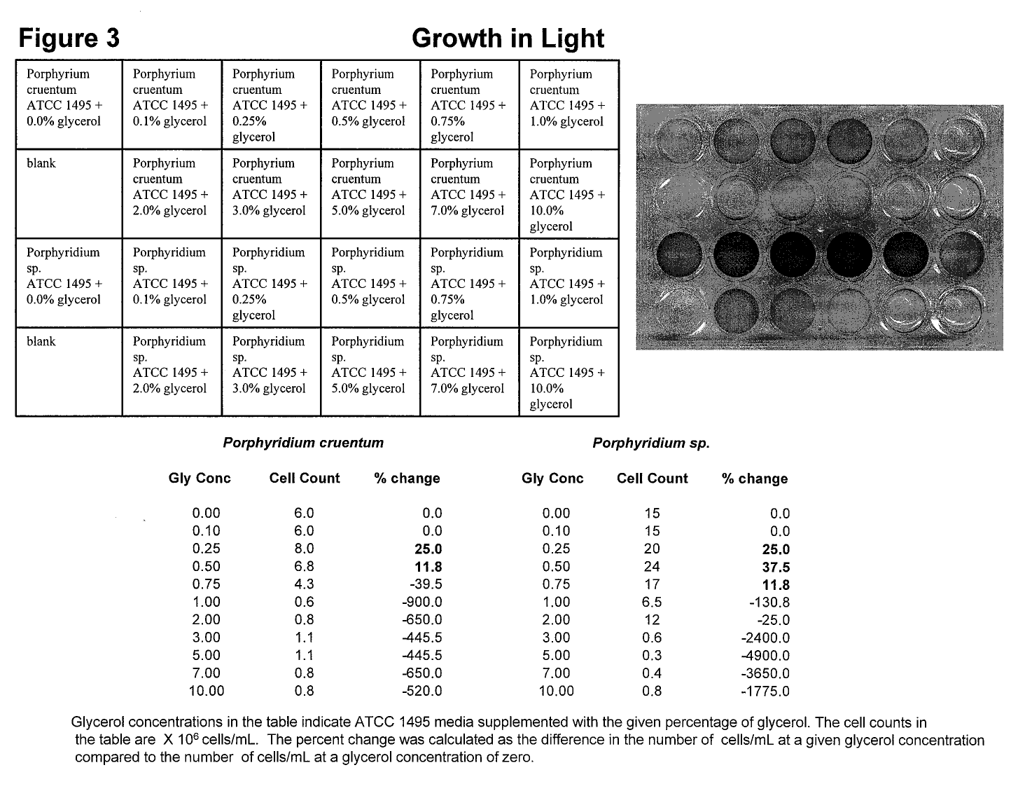

Cultures of Porphyridium sp. (UTEX 637) and Porphyridium cruentum (strain UTEX 161) were grown, to a density of 4×10⁶ cells/mL, as described in Example 1. For each strain, about 2×10⁶ cells/mL cells per well (˜500 uL) were transferred to 11 wells of a 24 well microtiter plate. These wells contained ATCC 1495 media supplemented with varying concentration of glycerol as follows: 0%, 0.1%, 0.25%, 0.5%, 0.75%, 1%, 2%, 3%, 5%, 7% and 10%. Duplicate microtiter plates were shaken (a) under continuous illumination of approximately 2400 lux as measured by a VWR Traceable light meter (cat #21800-014), and (b) in the absence of light. After 5 days, the effect of increasing concentrations of glycerol on the growth rate of these two species of Porphyridium in the light was monitored using a hemocytometer. The results are given in FIG. 3 and indicate that in light, 0.25 to 0.75 percent glycerol supports the highest growth rate, with an apparent optimum concentration of 0.5%.

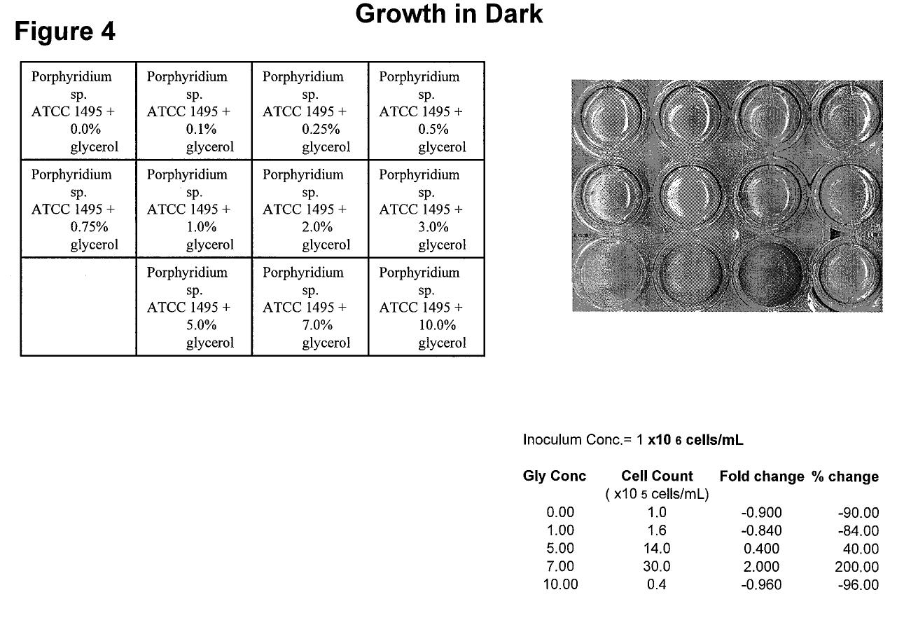

Cells in the dark were observed after about 2 weeks of growth. The results are given in FIG. 4 and indicate that in complete darkness, 5.0 to 7.0% glycerol supports the highest growth rate, with an apparent optimum concentration of 7.0%.

### Example 9

### Cosmetic Compositions

Porphyridium sp. (UTEX 637) was grown to a density of approximately 4×10⁶ cells/mL, as described in Example 1. Approximately 50 grams of wet pelleted, and washed cells were completely homogenized using approximately 20 minutes of sonication as described. The homogenized biomass was mixed with carriers including, water, butylene glycol, mineral oil, petrolatum, glycerin, cetyl alcohol, propylene glycol dicaprylate/dicaprate, PEG-40 stearate, C11-13 isoparaffin, glyceryl stearate, tri (PPG-3 myristyl ether) citrate, emulsifying wax, dimethicone, DMDM hydantoin, methylparaben, carbomer 940, ethylparaben, propylparaben, titanium dioxide, disodium EDTA, sodium hydroxide, butylparaben, and xanthan gum. The mixture was then further homogenized to form a composition suitable for topical administration. The composition was applied to human skin daily for a period of one week.

### Example 10

### Sexually Transmitted Disease Prevention Compositions

Polysaccharide from Porphyridium sp. was prepared as described in Example 2. Lyophilized polysaccharide was resuspended with distilled water to an antivirally effective concentration of 0.5 milligram per mL. 1.0 mL of the 0.5 mg/mL polysaccharide solution was applied to a latex condom.

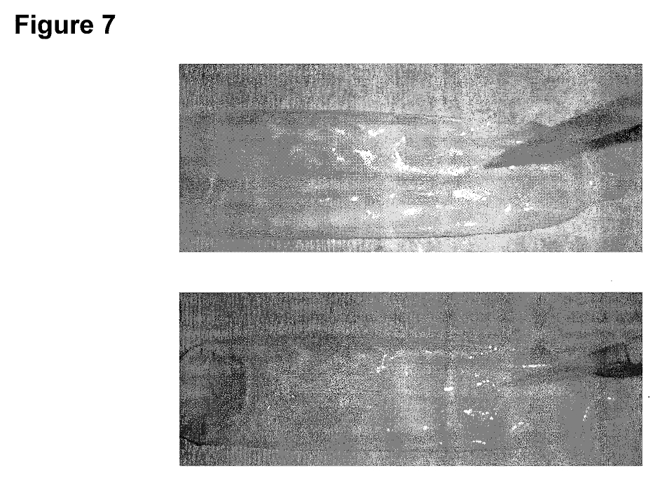

In a second composition formulation, 10 microliters of a 1 mg/mL Porphyridium sp. polysaccharide solution was applied to a latex condom. 29 additional 10 microliter increments of the 1 mg/mL solution were successively applied, creating individual sexually transmitted disease composition with between 100 micrograms and 3 milligrams of polysaccharide in 100 microgram increments. See FIG. 7.

Other condom formulation techniques can be used (see for example U.S. Pat. No. 6,196,227).

### Example 11

Approximately 4500 cells (300 ul of 1.5×10⁵ cells per ml) of Porphyridium sp. and Porphyridium cruentum cultures in liquid ATCC 1495 ASW media were plated onto ATCC 1495 ASW agar plates (1.5% agar). The plates contained varying amounts of zeocin, sulfometuron, hygromycin and spectinomycin. The plates were put under constant artificial fluorescent light of approximately 480 lux. After 14 days, plates were checked for growth. Results were as follows: (1) Zeocin: (Concentration μg/ml) [Growth]: (0.0) [++++]; (2.5) [+]; (5.0) [−]; (7.0) [−]. (2) Hygromycin (Concentration μg/ml) [Growth]: (0.0) [++++]; (5.0) [++++]; (10.0) [++++]; (50.0)[++++]. (3) Specinomycin (Concentration μg/ml) [Growth]: (0.0) [++++]; (100.0)[++++]; (250.0) [++++]; (750.0) [+++].

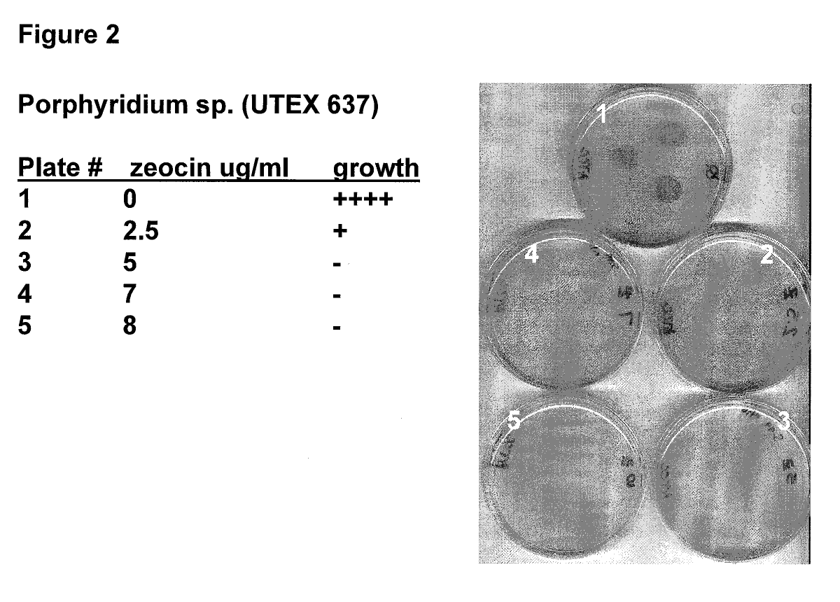

After the initial results above were obtained, a titration of zeocin was performed to more accurately determine growth levels of Porphyridium in the presence of zeocin. Porphyridium sp. cells were plated as described above. Results are shown in FIG. 2.

### Example 12

### Trophic Conversion

### Transporters

### Cloning

Plasmid pBluescript KS+ is used as a recipient vector for an expression cassette shown in SEQ ID NO:68. The Porphyridium glycoprotein promoter is cloned into pBluescript KS+, followed by a cauliflower mosaic virus 3′ UTR. Unique restriction sites are left between the promoter and 3′UTR. A nucleic acid encoding the glucose transporter of SEQ ID NO:20 using preferred codons of Porphyridium sp. is cloned into the unique restriction sites between the promoter and 3′UTR.

The plasmid is used to transform Porphyridium sp. cells using the biolistic transformation parameters described in Plant Physiol. 2002 May; 129(1):7-12. After transformation, some plated cells are scraped from the plate using a sterile cell scraper are transferred into Erlenmeyer flasks wrapped with aluminum foil sufficient to prevent the entry of light into the culture. Identical preparations of transformed, scraped cells are cultured, shaking at 50 rpm in 24 well plates in the dark, in ATCC 1495 media in the presence of 0.1, 1.0, and 2.5% glucose, and monitored for growth. Other cells are transformed on plates containing solid agar ATCC 1495 media, supplemented with either 0.1, 1.0, or 2.5% glucose, and monitored for growth in complete darkness.

### Example 13

Cultures of Porphyridium sp. (UTEX 637) and Porphyridium cruentum (strain UTEX 161) were subjected to chemical mutagenesis (from the protocol in Gorman D S, Levine R P. (1965) Proc Natl Acad Sci USA. 54(6):1665-9.). Cells were grown to a density of 4×10⁶ cells/mL as described in Example 1. Cells were harvested, washed with 70 mM potassium phosphate buffer (pH 6.9) and resuspended to a density of 4×10⁷ cells/mL. To 1 mL of cells (from both strains), 0.1M ethyl methane sulfonate (EMS) was added. A 200 uL aliquot was taken for the zero time point. The tubes were incubated in the dark at room temperature. 200 uL aliquots were removed from the tube at various time points: 15 min, 30 min, 45 min and 60 min. At each time, the aliquot of cells were treated with 800 uL of 5% sodium thiosulfate to inactivate the EMS. Cells from each aliquot were spun down and washed three times with 1 mL of 70 mM potassium phosphate buffer (pH 6.9), followed by a wash with 1 mL of ATCC 1495 media. The cells were resuspended in 200 uL of ATCC 1495 media, and plated at three different concentrations (1×, 10⁻²×, 10⁻⁴×) on duplicate plates of ATCC 1495 media, and incubated under continuous light.

After mutagenesis, some plated cells are scraped from the plate using a sterile cell scraper are transferred into Erlenmeyer flasks wrapped with aluminum foil sufficient to prevent the entry of light into the culture. Identical preparations of transformed, scraped cells are cultured, shaking at ˜50 rpm in 24 well plates in the dark, in ATCC 1495 media in the presence of 0.1, 1.0, and 2.5% glucose, and monitored for growth. Other cells are transformed on plates containing solid agar ATCC 1495 media, with either 0.1, 1.0, or 2.5% glucose, and monitored for growth in complete darkness. Cell treated as described can also be cultured in the presence of an exogenous carbon source from Tables 2 or 3.

### Example 14

### Genetic and Nutritional Manipulation to Generate Novel Polysaccharides

Cells prepared as described in Example 12 containing a monosaccharide transporter and capable of importing glucose, are cultured in ATCC 1495 media in the light in the presence of 1.0% glucose for approximately 12 days. Exopolysaccharide is purified as described in Example 2. Monosaccharide analysis is performed as described in Example 5.

Cells prepared as described in Example 12 containing a monosaccharide transporter and capable of importing xylose, are cultured in ATCC 1495 media in the light in the presence of 1.0% xylose for approximately 12 days. Exopolysaccharide is purified as described in Example 2. Monosaccharide analysis is performed as described in Example 5.

Cells prepared as described in Example 12 containing a monosaccharide transporter and capable of importing galactose, are cultured in ATCC 1495 media in the light in the presence of 1.0% galactose for approximately 12 days. Exopolysaccharide is purified as described in Example 2. Monosaccharide analysis is performed as described in Example 5.

Cells prepared as described in Example 12 containing a monosaccharide transporter and capable of importing glucuronic acid, are cultured in ATCC 1495 media in the light in the presence of 1.0% glucuronic acid for approximately 12 days. Exopolysaccharide is purified as described in Example 2. Monosaccharide analysis is performed as described in Example 5.

Cells prepared as described in Example 12 containing a monosaccharide transporter and capable of importing glucose, are cultured in ATCC 1495 media in the dark in the presence of 1.0% glucose for approximately 12 days. Exopolysaccharide is purified as described in Example 2. Monosaccharide analysis is performed as described in Example 5.

Cells prepared as described in Example 12 containing a monosaccharide transporter and capable of importing xylose, are cultured in ATCC 1495 media in the dark in the presence of 1.0% xylose for approximately 12 days. Exopolysaccharide is purified as described in Example 2. Monosaccharide analysis is performed as described in Example 5.

Cells prepared as described in Example 12 containing a monosaccharide transporter and capable of importing galactose, are cultured in ATCC 1495 media in the dark in the presence of 1.0% galactose for approximately 12 days. Exopolysaccharide is purified as described in Example 2. Monosaccharide analysis is performed as described in Example 5.

Cells prepared as described in Example 12 containing a monosaccharide transporter and capable of importing glucuronic acid, are cultured in ATCC 1495 media in the dark in the presence of 1.0% glucuronic acid for approximately 12 days. Exopolysaccharide is purified as described in Example 2. Monosaccharide analysis is performed as described in Example 5.

### Example 15

128 mg of intact lyophilized Porphyridium sp. cells were ground with a mortar/pestle. The sample placed in the mortar pestle was ground for 5 minutes. 9.0 mg of the sample of the ground cells was placed in a micro centrifuge tube and suspended in 1000 μL of dH2O. The sample was vortexed to suspend the cells.

A second sample of 9.0 mg of intact, lyophilized Porphyridium sp. cells was placed in a micro centrifuge tube and suspended in 1000 μL of dH2O. The sample was vortexed to suspend the cells.

The suspensions of both cells were diluted 1:10 and polysaccharide concentration of the diluted samples was measured by DMMB assay. Upon grinding, the suspension of ground cells resulted in an approximately 10-fold increase in the solvent-accessible polysaccharide as measured by DMMB assay over the same quantity of intact cells.

TABLE 12

| | Read 1 | Read 2 | Avg. Abs | Conc. |
|---|---|---|---|---|
| Sample Description | (AU) | (AU) | (AU) | (μg/mL) |
| Blank | 0 | −0.004 | −0.002 | 0 |
| 50 ng/μL Std., 10 μL; 0.5 μg | 0.03 | 0.028 | 0.029 | NA |
| 100 ng/μL Std., 10 μL; 1.0 μg | 0.056 | 0.055 | 0.0555 | NA |
| Whole cell suspension | 0.009 | 0.004 | 0.0065 | 0.0102 |
| Ground cell suspension | 0.091 | 0.072 | 0.0815 | 0.128 |

Reduction in the particle size of the lyophilized biomass by homogenization in a mortar/pestle results in better suspension and increase in the solvent-accessible polysaccharide concentration of the cell suspension.

### Example 16

Porphyridium cruentum was grown as described above in ATCC 1495 media. Porphyridium cruentum culture supernatant were autoclaved at 121° C. for 20 minutes. 1.333 liters of isopropanol was added to a 4 liter preparation of autoclaved supernatant to a concentration of 25% (vol/vol). Precipitated exopolysaccharide was removed. Additional isopropanol (381 mL, 786 mL, 167 mL, and 1.333 liters) was added stepwise to the preparation to produce (vol/vol) concentrations of isopropanol of 30%, 38.5%, 40%, and 50%, respectively. Precipitated exopolysaccharide was removed after each increment of isopropanol was added. It was observed that very little additional exopolysaccharide was precipitated upon bringing the concentration from 38.5% to 40% and from 40% to 50%. It was also observed that significant amounts of salt were precipitated upon bringing the concentration from 38.5% to 40% and from 40% to 50%.

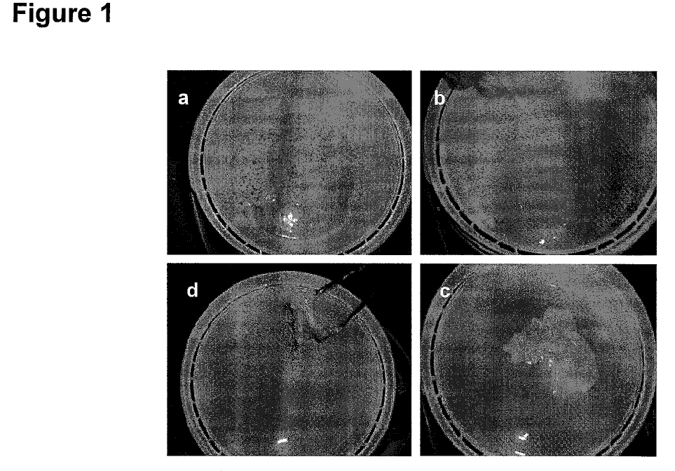

An additional 4 liters of exopolysaccharide was precipitated with by addition of 38.5% isopropanol. See FIG. 1.

### Example 17

Preparation of DNA templates for Inverse PCR: Porphyridium sp. genomic DNA (40 μg) was digested with SacI at 37° C. for 16 h in a total volume of 200 μl. The digested DNA was extracted with once an equal volume of phenol:chloroform isoamyl alcohol (PCI, 25:24:1) followed by one extraction with equal volume of chloroform. The DNA was precipitated with 1/10th volume of 3M sodium acetate, pH5.5 and 2.5 volumes of 100% ethanol. The DNA was pelleted by centrifugation, air dried and resuspended in 50 μl distilled water to a concentration of 250 ng/ul. Four dilutions of the DNA (1 μg; 100 ng; 10 ng; 1 ng) was self-ligated for 18 h at 16° C. in a 100 μl reaction volume using T4 DNA ligase (Invitrogen, Carlsbad, Calif.). The ligated DNA was extracted once with PCI followed by a chloroform extraction. The sample was ethanol precipitated as described above and resuspended in 200 ul distilled water in 4 individual aliquots at a concentration of 2.5 ng/μL; 250 pg/μL; 25 pg/μL; and 2.5 pg/μL. 1 μL of each of the 4 aliquots was used as template in subsequent inverse PCR reactions.

Inverse PCR: PCR was carried out in 25 μl volume using EXL polymerase (Stratagene, La Jolla, Calif.) in 0.2 ml thin-walled tubes. PCR reactions were carried out in two rounds. The first round of PCR was set up according to the manufacturer's instructions. The reaction contained 4% DMSO, 1 μL stabilizing solution and 0.25 μM of primers 1 and 2 (primer #1, SEQ ID NO:49, primer #2, SEQ ID NO:50). Inverse PCR primers 1 and 2 were designed in opposite orientation to that of normal PCR and were designed to anneal to the Porphyridium sp. glycoprotein cDNA sequence (GenBank Accession number AY778963). PCR cycle conditions were as follows: initial denaturation of 94° C. for 2 minutes followed by 35 cycles of 92° C. for 10 seconds, 60° C. for 30 seconds and 68° C. for 15 minutes, followed by a final extension at 68° C. for 5 minutes. The first round PCR product was diluted 100 fold, and 1 of the dilution was used as a template for a second round PCR. Second round PCR was done using nested primers 3 and 4 (primer #3, SEQ ID NO:51, primer #4, SEQ ID NO:52). PCR cycle conditions were as follows: initial denaturation of 94° C. for 2 minutes followed by 35 cycles of 92° C. for 10 seconds, 58° C. for 30 seconds and 68° C. for 6 minutes, followed by a final extension at 68° C. for 5 minutes.

Cloning of the Inverse PCR fragments: A 1.8 kB inverse PCR product was amplified from SacI-digested, self-ligated genomic DNA. The PCR product was cloned into the pGEM-T vector and transformed into JM109 high efficiency competent cells (Promega, Madison, Wis.) and selected on LB+Amp plates containing XGal/IPTG. Plasmid DNA extracted from white colonies was screened for inserts and confirmed to contain the 1.8 KB PCR product by sequencing with M13 forward and M13 reverse primers (SEQ ID NOs:53-54, respectively). Full length sequence of SEQ ID NO:44 was obtained by internal sequencing of the cloned 1.8 kB clone. As expected, the 3′ end of the clone was identical to the beginning of the coding region of the glycoprotein cDNA (GenBank Accession number AY778963).

The 1.8 kB promoter fragment was amplified by PCR and cloned into operable linkage with a zeocin resistance gene and the carbohydrate transporter cDNAs encoding the proteins of SEQ ID NOs:20, 22, 24, and 46-48 to generate 7 unique Porphyridium transformation vectors.

### Example 18

The promoter of SEQ ID NO:55 was cloned into operable linkage with a zeocin resistance cDNA encoding the protein of SEQ ID NO: 11. The zeocin resistance cDNA was in operable linkage with the CMV 3′ UTR of PCT application WO2006013572. The plasmid was linearized by a restriction enzyme that did not cut in the promoter, cDNA, or 3′UTR.

Porphyridium sp. (strain UTEX 637) culture was grown in ATCC 1495 medium on a gyratory shaker under continuous light at 75 μmol photons m⁻² sec⁻¹ until it reached a cell density of 2×10⁶ cells/mL, following which they were incubated in the dark for 24 hours. The dark adapted cells were harvested, and washed twice with sterile distilled water, and resuspended in a Tris-phosphate buffer (20 mM Tris-HCl, pH 7.0; 1 mM potassium phosphate) containing 50 mM sucrose to a density of 4×10⁸ cells/mL. About 250 μL cell suspension (1×10⁸ cells) was placed in a disposable electroporation cuvette of 4 mm gap. To the cell suspension, 2-5 μg of linearized plasmid DNA and 200 μg of carrier DNA (sheared salmon sperm DNA) were added. The electroporation cuvette was then incubated in a water bath at 16° C. for 10 minutes. An electrical pulse (1800 V/cm) was then applied to the cuvette at a capacitance of 25 μF (no shunt resistor was used for the electroporation) using a Gene Pulser II (Bio-Rad Labs, Hercules, Calif.) electroporation apparatus. The cuvette was then incubated at room temperature for 5 minutes, following which the cell suspension was transferred to 50 mL of ATCC 1495 media, and shaken on a gyratory shaker for 2 days. Following recovery, the cells were harvested at low speed (4000 rpm), resuspended in distilled water and plated out at low density on solid media (ATCC 1495+1% agar) supplemented with 30 μg/mL zeocin (Invitrogen, Carlsbad, Calif.). The plates were incubated under continuous light at 75 μmol photons m⁻² sec⁻¹. Transformants appeared as colonies in 2-3 weeks.

### Example 19

### Animal Model

The effect of disclosed compositions on total serum cholesterol was assessed with an animal model. Golden Syrian male hamsters weighing between 100 and 120 g were used in this experiment. Animals were randomized into 7 groups of 15 hamsters per group, housed in individual cages and subjected to 12 hr: 12 hr light:dark cycling for 2 week before commencement on the experimental protocol. During the pre-experimental and experimental periods, hamsters were provided with free access to water, and fed ad libitum a semi-synthetic diet (control diet), based on the composition of the AIN-76A which contains 0.25% cholesterol (see Table 13 below).

TABLE 13
Composition of basal semi-purified diet

| Ingredients | % (wet weight) |
|---|---|
| Casein | 20.0 |
| Corn starch | 28.0 |
| Sucrose | 36.3 |
| Lard/safflower oil | 5.0 |
| Cellulose | 5.0 |
| D-methionine | 0.5 |
| Mineral mixture | 4.0 |
| Vitamin mixture | 1.0 |
| Choline bitartrate | 0.2 |
| Cholesterol | 0.25 |
| BHT | 0.02 |

The total fat content of the control diet was 5% fed in the form of a mix of lard and safflower oil to provide a P/S ratio of 0.4. The control diet alone will be fed to Group 1. For identity of diets in Groups 2-8, see FIG. 10 (a). Biomass and polysaccharide was matrixed into the oil component of the diets. Food intake and body weight of individual animals was monitored regularly through the feeding period.

For diets listed as Quadro ground, biomass was homogenized in a Quadro F10 rotor homogenizer (Quadro Engineering Inc., Waterloo, Ontario, Canada). For ball milled samples: samples were cryogenically ball milled in a planetary ball mill (Retsch, PM100) at 10-80 g batch size. The powder was placed in a grinding bowl with eight to ten ¾-inch-diameter stainless steel balls. The sample was cooled with liquid nitrogen repeatedly. The material was milled at 400-550 rpm for 30 to 60 min. The final product was dried in a desiccator overnight.

After 30 days on diets, hamsters in each group will be anaethesized with halothane and blood samples were collected and stored at −80° C. for determination of total cholesterol, low density lipoprotein (LDL), and high density lipoprotein (HDL) subclass cholesterol and triglyceride (TG) levels. Measures of antioxidant status were also determined through TBARs assays.

### Lipid Analysis

Plasma total cholesterol was measured using a VG Autoanalyzer in conjunction with commercial enzymatic kits and appropriate standards (Abbott Diagnostics, Montreal, Quebec). All samples were processed through the above system in duplicate.

The results are shown in FIG. 10 (b) and (c), where each of Groups 2-8 exhibited a decrease in total serum cholesterol (TSC) levels in comparison to the control diet (Group 1). The lowering of total serum cholesterol levels ranged from about 35 to about 62%.

The results reflect an unexpected discovery compared to earlier reports. For example, Dvir et al. reported only a 21.8% reduction in TSC by feeding rodents microalgal biomass at 19% of the total diet. This is in contrast to the 5% of the total diet used in Group 3 and the 10% of the total diet in Group 5 of the instant experiment, which produced reductions of 35.9% and 54.2%, respectively. The results from the instant experiment are also unexpected relative to the report by Ginzberg et al., where 5% and 10% diets only produced 11% and 28% reductions in TSC, respectively. The results from Group 7 (62% reduction) also indicate the presence of unexpected synergy in a combination of cell homogenate and an additional agent such as phytosterol.

### Example 20

The effect of disclosed compositions on anthropometric indices was studied in the hamsters described in Example 19.

Body weight was measured once a week and food consumption once every two days by weighing the difference of food cups before and after the feeding. Measurements of body composition were performed using DEXA analyses at sacrifice. Methods to determine body composition and energy expenditure (metabolism) were performed essentially as described by Lei et al. (“Relationship of total body fatness and five anthropometric indices in Chinese aged 20-40 years: different effects of age and gender” Eur. J. Clin. Nutri. (2006) 60:511-518) and Nagao et al. (“The 10trans, 12 cis isomer of conjugated linoleic acid promotes energy metabolism in OLETF rats” Nutri. (2003) 19:652-656).

### Increasing Satiety

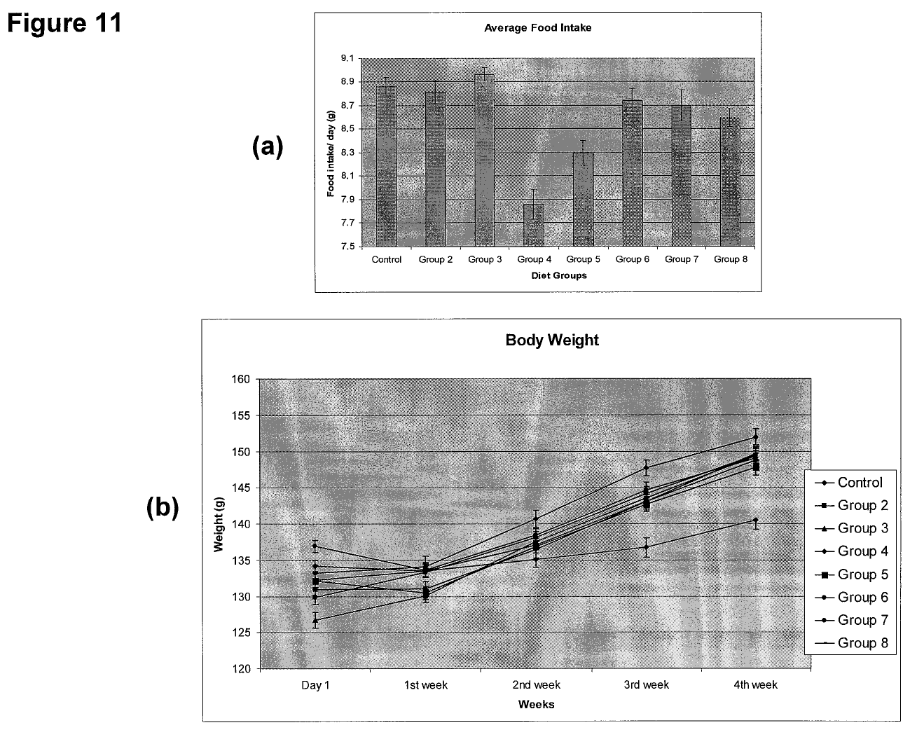

The average food intake results are shown in FIG. 11 (a), where some of Groups 2-8 exhibited a decrease in average food intake in comparison to the control diet (Group 1). The results reflect an increased satiety, and so decreased appetite, seen in at least the hamsters of Groups 4 and 5. The effect on body weight is shown in FIG. 11 (b). While the invention is not limited by theory, it is believed that the propensity of the polysaccharide to swell in size on contact with water in the gut, thus signaling a feeling of satiety in mammals.

### Body Composition

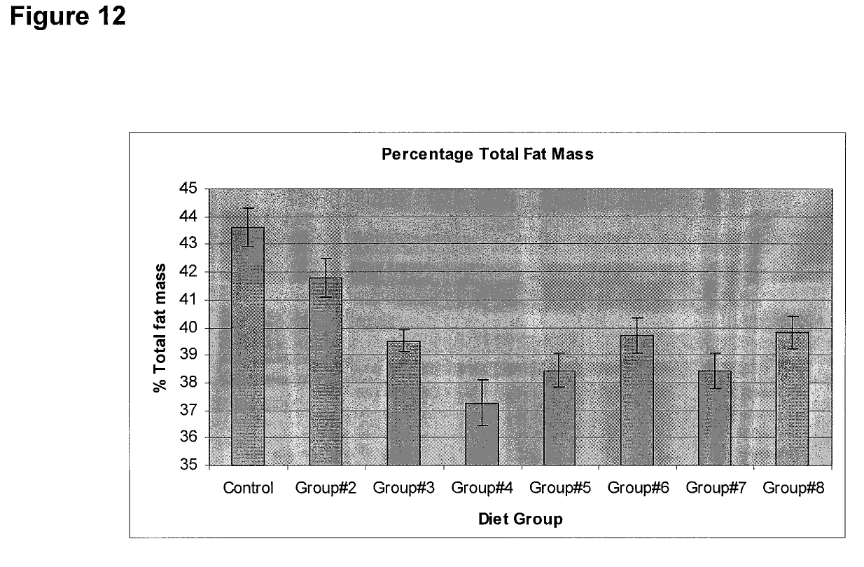

The body composition (total fat mass) results are shown in FIG. 12, where each of Groups 2-8 exhibited a decrease in total body fat mass in comparison to the control diet (Group 1). The results reflect a decrease in body fat (or hypolipidemic effect) seen in the hamsters of Groups 2-8.

### Energy Expenditure

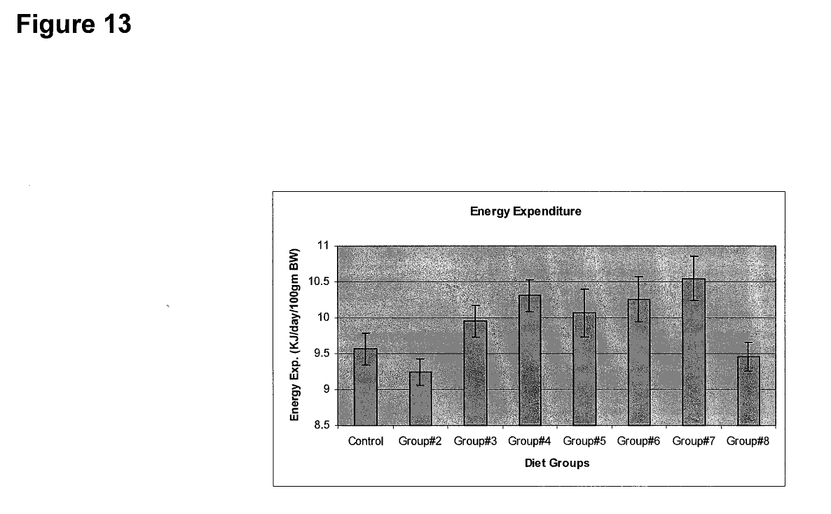

The energy expenditure results are shown in FIG. 13, where some of Groups 2-8 exhibited an increase in energy expenditure in comparison to the control diet (Group 1). The results reflect increased energy use (and oxygen consumption) in at least the hamsters of Groups 4, 6 and 7.

### Example 21

### Antioxidant Status

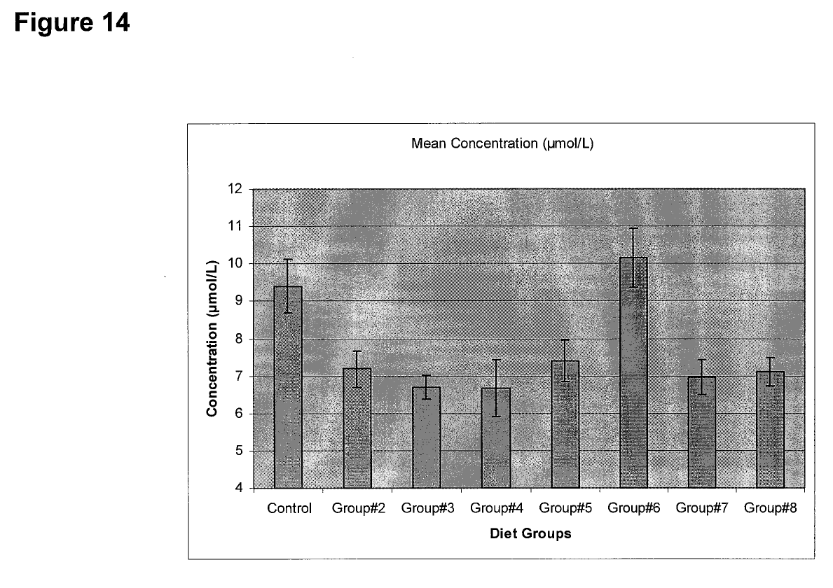

The effect of disclosed compositions on antioxidant status was studied in the hamsters described in Example 19. Measurement of TBARs in frozen plasma were conducted using standardized commercially available kits (Catalog #: 0801192, ZeptoMetrix Corporation, Buffalo, N.Y.), against appropriate standards. The plasma samples were frozen after collection until used to determine oxidative status by measuring thiobarbituric acid-reactive substances (TBARs). These analyses were conducted in duplicate to reduce analytical variability associated with these measurements. The results are shown in FIG. 14, where each of Groups 2-5, 7, and 8 displayed a decrease in TBARs in comparison to the control diet (Group 1).

A comparison of the results for Groups 2 and 6, for example, indicate an unexpected discovery that homogenization or disruption of the cells (biomass), even when administered at the same dosage, results in an increase an antioxidant status in the plasma of the treated animals. Given an understanding of the microalgal polysaccharides as on the surface of the cells or extracellular, there was no expectation that rupturing the microalgal cells in a feed composition fed to animals would increase the antioxidants in the animal. However, the results clearly indicate the superiority of homogenized or disrupted biomass (cells).

These results demonstrate that compositions provided herein are useful, for example, in alleviating age-associated cognitive and motor changes in mammals. See for example Brain Res. 1995 Sep. 25; 693(1-2):88-94.

### Example 22

A Porphyridium sp. biomass homogenate was tested for COX1 and COX2 inhibition activity. Porphyridium sp. biomass was grown as described in Example 1, recovered by centrifugation, washed with deionizer water to remove salts, and centrifuged again to form a pelleted paste. Nitrogen was bubbled through the paste for 30 minutes to displace dissolved oxygen and minimize subsequent oxidation. The paste was then passed through a model 110Y Microfluidics Microfluidizer® at 22,000 PSI with cooling, and the process repeated until at least 50% of the cells were broken as determined by microscopic examination. Nitrogen was once again bubbled through the paste, which was then lyophilized after shell freezing in dry ice ethanol. The dried cell mass was then ground to a fine powder with a Braun® kitchen homogenizer.

The powder was analyzed for inhibition of constitutive cyclooxygenase (COX1) and inducible cyclooxygenase (COX2) at 37° C. by monitoring oxygen consumption using an Oxytherm Electrode Unit by Hansatech. The IC₅₀ values are equal to the concentration of the sample that inhibits 50% activity of the cyclooxygenase in the reaction mixture. The results are shown in Table 14.

TABLE 14

| | COX1 IC₅₀ | COX2 IC₅₀ | Ratio of |
|---|---|---|---|
| Sample | (mg/ml) | (mg/ml) | COX1:COX2 values |
| Homogenized | 0.15 | 0.17 | 0.88 |
| Porphyridium sp. | | | |
| biomass powder | | | |

Results indicate surprisingly significant anti-inflammatory activity, particularly for a natural product.

Certain aspects of the invention are based upon the discovery of an anti-inflammatory effect in animals that ingest the cell material (biomass, homogenate, and/or polysaccharides) described herein. While the basis of the anti-inflammatory effect has yet to be completely elucidated, the materials of the invention both increase the antioxidant status of mammalian plasma (see Example 23 below) and inhibit cyclooxygenase activity. The cell material has anti-inflammatory activity demonstrated through multiple methods of analysis and shown to be exerted through independent meachansism that would not have been expected.

Animal models that are widely viewed to reflect inflammatory responses and to have predictive value in assessing the efficacy of various treatments for these disorders can be utilized to evaluate the therapeutic efficacy of the compounds described herein. The effects of the compounds of the invention on joint inflammation and lameness can be assessed in horses with trauma-induced inflammation. Alternatively, improvement of inflammation and mobility can be measured with a collagen-induced rheumatoid arthritis mouse model (see, Enokida M et al. (2001) Bone 28(1): 87-93)

### Example 23

Two filter-purified polysaccharide preparations (containing 3.57% and 4.23% sulfur by weight), prepared as described in Example 24, were subjected to oxygen radical absorption capacity (ORAC) analysis as described in Journal of Agricultural and Food Chemistry.; 2001; 49(10); 4619-4626 and Journal of Agricultural and Food Chemistry.; 2002, 50(7); 1815-1821. The polysaccharide containing 3.57% sulfur by weight did not exhibit ORAC activity. The polysaccharide containing 4.23% sulfur by weight exhibited ORAC activity of 2.0 trolox equivalents per gram.

### Example 24

Porphyridium cruentum and Porphyridium sp. were grown in artificial seawater media essentially as described in Example 1 except that the amount of MgSO₄ was varied. Porphyridium sp. cells were grown in 17 mM MgSO₄. Porphyridium cruentum was grown in 120 mM, 600 mM, 750 mM, 1M, and 2M MgSO₄. Cell division occurred at all concentrations. Polysaccharide was purified essentially as described in Example 2 from the 120 and 600 mM cultures, to the point where all soluble protein and small molecules were removed. Sulfur content was analyzed according to US EPA SW846, Method 6010B, Inductively Coupled Plasma-Atomic Emission Spectrometry. The polysaccharide purified from the 17, 120 and 600 mM cultures contained 3.57, 4.23 and 5.57% sulfur, respectively. As disclosed herein, these results reflect an unexpected discovery because the skilled person would not expect that the microalgae would survive in media containing more than 100 mM sulfate.

### Example 25

### Transformation of Porphyridium and Genotyping

The Porphyridium glycoprotein promoter, SEQ ID NO:44, was cloned in operable linkage with a zeocin resistance ble cDNA containing small 5′ and 3′ flanking regions (SEQ ID NO: 70) encoding the protein of SEQ ID NO: 11, with the far 5′ region of the glycoprotein promoter directly adjacent to the pBlusecript SacI site and the far 3′ region of the CMV 3′UTR (SEQ ID NO:69) adjacent to the pBlusecript KpnI site. The CMV 3′UTR was also in operable linkage with the ble cDNA. The plasmid was linearized by KpnI, which does cut in the promoter, ble cDNA, or 3′UTR, prior to transformation.

The biolistic particle delivery system PDS 1000/He (Bio-Rad, USA) was used for transformation. Porphyridium sp. culture was grown to logarithmic phase (˜2×10⁶ cells/mL) in liquid ATCC 1495 media under continuous light (approximately 75 umol/photons/m²). Cells from this culture were harvested at 4,000 rpm at room temperature. The cell pellet was washed twice with sterile distilled water. Cells were resuspended in fresh ATCC 1495 media to the same cell density i.e. ˜2×10⁶ cells/mL and incubated in the dark for 16 hours. The dark adapted cells were then harvested at 4000 rpm at room temperature, resuspended in fresh ATCC 1495 media to a density of ˜2×10⁸ cells/mL. Approximately 1×10⁸ cells were transferred to each ATCC 1495 agarose plate. Filter sterilized DNA from the plasmids was coated onto 550 nm gold particles (catalog number S04e, Seashell Technology, USA) according to the manufacturer's protocol. For each of the particle bombardments, 1 ug of plasmid DNA was used. The negative controls were bombarded in identical fashion with gold particles coated with a plasmid containing the Porphyridium glycoprotein promoter, SEQ ID NO:44, and the CMV 3′UTR, (SEQ ID NO:69), with no zeocin resistance gene. Each of the particle bombardments were performed using 1350 psi rupture disks, at bombardment distance of 9 cm, and under 28 in. Hg vacuum. The bombarded cells were scraped off the plates, and transferred to 100 ml of fresh ATCC 1495 media, and shaken under continuous light (approximately 75 umol/m²) for 3 days. Following recovery, the cells were harvested at 4,000 rpm at room temperature, and plated onto ATCC 1495 plates supplemented with 30 ug/mL Zeocin (Invitrogen, Carlsbad, Calif., USA) at a cell density of 1×10⁷ cells/plate. These plates were incubated under light (approximately 25 umol/m²) for 4-5 weeks. Zeocin resistant colonies growing on these plates were scored as transformants and transferred onto fresh ATCC 1495 plates supplemented with 30 ug/mL Zeocin (Invitrogen, Carlsbad, Calif., USA) for growth and analysis.

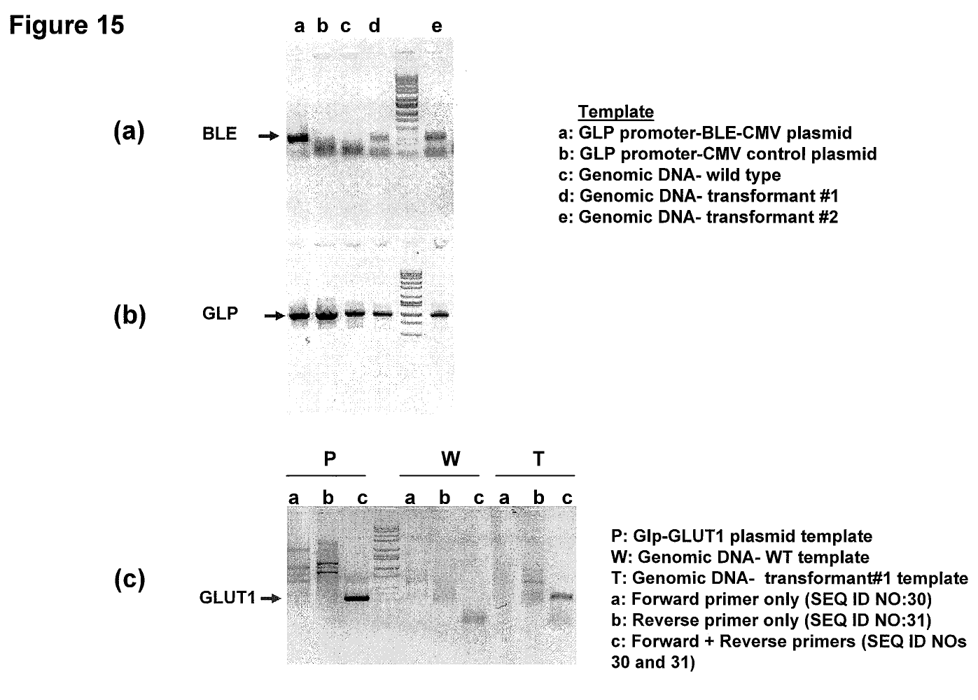

Zeocin resistant colonies appeared after 2-3 weeks. Genotyping with primers specific to the zeocin resistance gene was performed on genomic DNA isolated from zeocin resistant colonies. Results from genotyping of one strain (referred to herein and labeled as “transformant #2” in FIG. 15) indicated that the zeocin resistance gene was present. A band of the correct size was amplified. Results are shown in FIG. 5 and discussed in more detail in Example 21.

### Example 26

### Transformation of Porphyridium, Genotyping, and Southern Blot Analysis

The zeocin resistance plasmid described in Example 20 and a second plasmid that was identical with the exception that it contained a cDNA for a human GLUT1 glucose transporter (SEQ ID NO:25) instead of the ble cDNA were combined in a co-transformation experiment carried out essentially as described in Example 20 except that both the zeocin resistance and GLUT1 plasmids were both adhered to the gold beads. A zeocin resistant colony (referred to herein as transformant#1) was selected for further analysis. Genomic DNA was extracted from wild type Porphyridium sp. and transformant#1.

Genotyping was performed on genomic DNA extracted from wild type, transformant#1, and transformant#2 DNA with plasmid DNA used as a template positive control and water in place of DNA as a template negative control. A segment of the Porphyridium glycoprotein (GLP) gene promoter was used as a target positive control. The following primer sets were used for the genotyping PCR: Ble-FWD (SEQ ID NO: 62) and Ble-REV (SEQ ID NO: 63), GLP-FWD (SEQ ID NO: 64) and (SEQ ID NO: 65), GLUT1-FWD (SEQ ID NO: 66) and GLUT1-REV (SEQ ID NO: 67). The PCR profile used was as follows: 94° C. denaturation for 5 min; 35 cycles of 94° C. for 30 sec, 51° C. or 60° C. (51° C. for glycoprotein gene & GLUT1 and 60° C. for ble) for 30 sec, 72° C. for 2 min; 72° C. for 5 min. Results are shown in FIG. 15. FIG. 15(a) demonstrates that the ble gene was present in both transformants, as the expected 300 bp product was generated. FIG. 15(b) demonstrates that the genomic DNA extraction and amplification was working, as the expected 948 bp glycoprotein promoter fragment was generated. FIG. 15(c) demonstrates that the GLUT1 gene was present transformant #1, as the expected 325 bp product was generated. DNA ladder was from BioNexus, Inc., All Purpose Hi-Lo DNA Marker, Catalog No: BN2050.

Specific bands can be amplified from residual plasmid DNA adhered to the outside of cells on transformation plates. Additionally, plasmids that have not been linearized can be maintained as episomes for a period of time before being degraded and can serve as template during PCR despite not having been integrated into a chromosome of a host organism. In both cases, microalgal strains may genotype positive despite the absence of stable chromosomal integration of the vector. Antibiotic resistant strains are known to arise due to mutagenesis caused by chromosomal damage from biolistic particles, electroporation conditions, and random genetic variation that is known to occur in microbial organisms. Southern blot analysis was performed to conclusively confirm the integration of the GLUT1 construct into the genome of transformant #1.

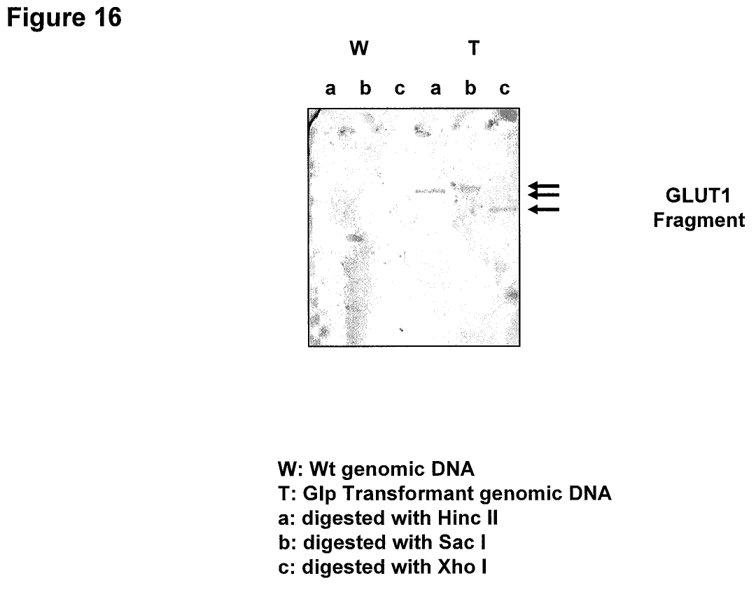

Southern blot analysis was performed on transformant #1. 20 μg genomic DNA from wild type and transformant#1 were individually digested with Hinc II, Sac I, Xho I and separated on a 1% agarose gel. DNA was transferred onto Nylon membrane (Hybond N+, Amersham Biosciences). A 1495 bp fragment containing the entire coding region of the GLUT1 gene was used as a probe. DIG labeled probes were generated for each probe fragment using the DIG High Prime DNA labeling and detection Kit according to the manufacturers instructions (Roche). The hybridizing bands were detected using the colorimetric detection reagents provided by the manufacturer. FIG. 16 demonstrates that the GLUT1 plasmid was stably integrated into the genome of transformant#1 while no detectable signal arose from wild type genomic DNA. As would be expected for a plasmid integrating into a chromosome of an organism, the specific band was in a different position for each different restriction enzyme used to digest the genomic DNA. This data conclusively demonstrates a species of the genus Porphyridium containing an exogenous gene encoding a carbohydrate transporter integrated into a chromosome of the organism. In this embodiment the carbohydrate transporter gene is in operable linkage with a promoter endogenous to a species of the genus Porphyridium. In some other embodiments embodiment the carbohydrate transporter gene is in operable linkage with a promoter endogenous to a Rhodophyte.

### Example 27

### Hyaluronidase Inhibition

Biotinylated hyaluronic acid (bHA) was covalently attached to the wells of a 96-well plate. Samples containing hyaluronidase and various test materials were then added to the wells. The hyaluronidase degrades the bound hyaluronic acid, resulting in a decrease in the amount of biotin covalently linked to the well plate. At the end of the incubation period the reaction was stopped and the well plate was washed to remove the hyaluronidase. The remaining bHA was detected using an avidin bound peroxidase enzyme. When an appropriate substrate is added, the peroxidase enzyme generated a color signal in proportion to the amount of bHA. The color signal was measured spectrophotometrically, and was inversely proportional to the amount of hyaluronidase activity in the sample. Thus, materials that inhibited hyaluronidase resulted in a greater color signal, since more of the bHA remained intact. Also see Frost, G., I., Stern, R. A Microtiter-Based Assay for Hyaluronidase Activity Not Requiring Specialized Reagents. Analytical Biochemistry 251, 263-269: 1997.

Preparation of Test Material Extracts

Test Material A was supplied as a powder type material. For this study, 100 mg of this material was combined with either 5 ml of ethanol or 5 ml of ultrapure water in 15 ml centrifuge tubes. After combining, the mixtures were vortexed, then placed onto a rocking platform for approximately 30 minutes at room temperature, and then centrifuged at 1,000 RPM for 5 minutes. The supernatants were then used at the final concentrations listed in the results section. Test Material B was supplied as a thick, viscous solutions (3%).

Anti-Hyaluronidase Assay: Immobilization of bHA onto 96-Well Plates

A solution of sulfo-NHS (0.184 mg/ml) and bHA (0.2 mg/ml) was prepared in distilled water. 50 μl of this solution was then added to the wells of a 96-well Covalink-NH plate. A solution of EDC (0.123 mg/ml) was then prepared in distilled water and 50 μl of this solution was added to each well (which resulted in a final concentration of 10 μg/well bHA and 6.15 μg/well of EDC). The well plate was then incubated overnight at 4±2° C. or for 2 hours at room temperature. After the incubation the plate was washed three times with PBS containing 2 M NaCl and 50 mM MgSO₄.

Anti-Hyaluronidase Assay

Prior to the assay, the well plate was equilibrated with assay buffer (0.1 M formate [pH 4.5], 0.1 M NaCl, 1% Triton X-100, 5 mM saccharolactone). The test materials were prepared in assay buffer at 2× their final concentration (heparin was used as a positive control, 1 mg/ml final concentration). After removing the assay buffer from the well plate used for equilibration, 50 μl of each of the prepared test materials was added to three wells on the well plate, followed by the addition of 50 μl of assay buffer containing hyaluronidase will be added to each well (0.1 mg/ml). Three additional wells were treated with 100 μl of assay buffer alone (without test materials and without hyaluronidase) and served as an index of zero hyaluronidase activity. After the addition of the test materials and enzyme, the plate was incubated for 30 minutes at room temperature. At the end of the incubation period, 200 μl of 6 M guanidine-HCl was added to each well to terminate the reaction. The plate was then washed three times with PBS containing 2 M NaCl, 50 mM MgSO₄ and 0.05% Tween 20.

During the 30 minute incubation, an avidin/biotin-peroxidase complex was prepared in 10.5 ml of PBS containing 0.1% Tween 20 using an ABC kit. This mixture was incubated for at least 30 minutes prior to use. After the plate was washed, 100 μl of the avidin/biotin-peroxidase solution was added to each well and the plate was incubated for another 30 minutes at room temperature. The plate was washed three times with PBS containing 2 M NaCl, 50 mM MgSO₄ and 0.05% Tween 20. After the final wash, 100 μl of substrate solution (one 10 mg tablet of OPD in 10 ml of 0.1 M citrate-PO₄ buffer supplemented with 7.5 μl of 30% H₂O₂) was added to each well. The plate was incubated in the dark for 10-20 minutes and then read at 460 nm using a plate reader. The substrate solution was also added to three wells that were not treated with test materials or the avidin/biotin-peroxidase solution and were used as a blank for the absorbance measurements.

TABLE 13

| Treatment | Percent Inhibition |
|---|---|
| 10% A Water Extract | 86 |
| 5% MATERIAL A Water Extract | 67 |
| 1% MATERIAL A Water Extract | 29 |
| 1.5% MATERIAL B | 93 |
| 0.5% MATERIAL B | 81 |
| 0.1% MATERIAL B | 70 |
| 0.1% Heparin | 74 |
| Negative Control | 0 |

Test Material Identification: MATERIAL A: Porphyridium sp. biomass homogenized (Quadro F10); cells grown as described in Example 1
Physical Description: Red/Purple powder
Concentrations Tested: 10%, 5%, 1% (Extracted in either ethanol or water)
Test Material Identification: 3% MATERIAL B: Exopolysaccahride from Porphyridium sp. purified as described in Example 2
Physical Description: Light tan, viscous liquid
Concentrations Tested: 1.5%, 1%, 0.5%, 0.1%

TABLE 15
Beta-carotene hydroxylases
Genbank accession number

NP_682690
YP_172377
NP_440788
YP_475340
YP_475340
BAB75708
ZP_01084736
ZP_01080915
ZP_01123496
ABB27016
NP_895643
NP_896386
ABB34062
YP_292794
AAP99312
ZP_01006041
ABB49299
NP_848964
ZP_01040435
NP_049051
YP_457405
AAT38625

TABLE 16
Superoxide dismutases
Genbank accession number

CAA42737
CAA43859
AAA29934
BAA02655
NP_625295
AH004200
CAC14367
YP_001003721
ABM60577
CAM08755
YP_966270
YP_963716
ABM37237
ABM35060
ABM33234
ABM33141
NP_407488
NP_405922
YP_932026
ZP_01641300
ZP_01638301
ZP_01637188
EAX24391
EAX23794
EAX23720
EAX23627
EAX20859
EAX19390
EAX16596
CAL93139
YP_914326
YP_747424
ABI59459
ZP_01610569
ZP_01605216
ZP_01600343
ZP_01584712
ZP_01581863
ZP_01581157
ZP_01575777
ZP_01569848
ZP_01559998
EAW01367
EAW01065
EAV97274
EAV95856
EAV80568
EAV73624
EAV73531
EAV70130
EAV66456
EAV61854
ZP_01532079
ZP_01516088
EAV26209
YP_845889
YP_822355
YP_843115
YP_836186
ABK17454

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-135. (canceled)

136. A composition comprising:
a. polysaccharide isolated from a cell of the genus Porphyridium; and
b. hyaluronic acid.

137. The composition of claim 136, wherein the composition is substantially free of endotoxins.

138. The composition of claim 136, wherein the composition is completely free of endotoxins.

139. The composition of claim 136, wherein the composition is substantially free of protein.

140. The composition of claim 136, wherein the composition is completely free of protein.

141. The composition of claim 136, wherein the cell is selected from the group consisting of Porphyridium sp. UTEX 637, a strain derived from Porphyridium sp. UTEX 637, Porphyridium cruentum UTEX 161, a strain derived from Porphyridium cruentum UTEX 161, Porphyridium aerugineum, a strain derived from Porphyridium aerugineum, Porphyridium sordidum, a strain derived from Porphyridium sordidum, Porphyridium purpureum, and a strain derived from Porphyridium purpureum.

142. The composition of claim 136, wherein the weight:weight ratio of hyaluronic acid to polysaccharide isolated from a cell of the genus Porphyridium is selected from the group consisting of at least 10,000:1, at least 1,000:1, at least 500:1, at least 250:1, at least 125:1, at least 100:1, at least 50:1, at least 10:1, at least 5:1, at least 4:1, at least 3:1, at least 2:1, at least 1.5:1, approximately 1:1, at most 1:10,000, at most 1:1,000, at most 1:500, at most 1:250, at most 1:125, at most 1:100, at most 1:50:1, at most 1:10, at most 1:5, at most 1:4, at most 1:3, at most 1:2; and at most 1:1.5.

143. The composition of claim 136, wherein the composition is sterile.

144. (canceled)

145. The composition of claim 136, wherein the composition further comprises a carrier suitable for sub-dermal administration.

146. The composition of claim 136, wherein the composition further comprises a carrier suitable for dermal administration.

147. The composition of claim 136, wherein the polysaccharide isolated from a cell of the genus Porphyridium contains an amount of sulfur by weight selected from the group consisting of at least 3%, at least 3.5%, at least 4.0%, at least 4.5%, at least 5.0%, at least 5.5%, at least 6.0%, at least 6.5%, at least 7.0%, at least 7.5% and at least 8.0%.

148. A method of extending the half life of a composition comprising hyaluronic acid comprising co-administering polysaccharide isolated from a cell of the genus Porphyridium.

149. The method of claim 148, wherein the hyaluronic acid and polysaccharide isolated from a cell of the genus Porphyridium are in direct contact before the co-administration.

150-151. (canceled)

152. The method of claim 148, wherein the composition is injected into a mammalian joint.

153. (canceled)

154. The method of claim 152, wherein the joint is selected from the group consisting of knee, shoulder, elbow, hip, ankle, wrist, and vertebrae.

155. The method of claim 148, wherein the composition is injected into skin.

156. The method of claim 155, wherein the skin is on a human face.

157-306. (canceled)

307. A combination product comprising:
a. a first composition comprising a microalgal polysaccharide and a carrier suitable for topical application; and
b. a second composition comprising at least one compound and a carrier suitable for human consumption;
wherein the first and second compositions are packaged for sale as a single unit.

308. The combination product of claim 307, wherein the first and second compositions contain at least one compound in common.

309. The combination product of claim 308, wherein the at least one compound is selected from the group consisting of DHA, EPA, ARA, lycopene, lutein, beta carotene, zeaxanthin, linoleic acid, vitamin C, a polysaccharide, a microalgal homogenate, and superoxide dismutase.

310. The combination product of claim 307, wherein the microalgal polysaccharide is produced from a cell of the genus Porphyridium.

311-360. (canceled)

361. A method of lubricating the joint of a mammal, comprising injecting a polysaccharide produced by microalgae into a cavity containing synovial fluid of the mammal.

362. (canceled)

363. The method of claim 361, wherein the microalgae is of the genus Porphyridium and the polysaccharide is an exopolysaccharide that is substantially free of protein and sterile.

364-469. (canceled)

470. A recombinant nucleic acid comprising at least 25 nucleotides of SEQ ID NO:44.

471-478. (canceled)

479. A recombinant nucleic acid comprising a first segment that has at least 25% nucleotide identity with any segment of SEQ ID NO:44, wherein the first segment is capable of initiating transcription in a Rhodophyta cell.

480. The recombinant nucleic acid of claim 479, wherein the first segment has a percent nucleotide identity with any segment of SEQ ID NO:44 selected from the group consisting of 35%, 45%, 55%, 65%, 75%, 85%, 95% and 98%.

481-483. (canceled)

484. The recombinant nucleic acid of claim 479, wherein the first segment is capable of initiating transcription in a Porphyridium cell.

485-486. (canceled)

487. A cell of the genus Porphyridium containing a recombinant copy of any segment of SEQ ID NO:44.

488. The combination product of claim 307, wherein the second composition contains at least one carotenoid.

489. The combination product of claim 307, wherein the second composition contains a polyunsaturated fatty acid.

490. The combination product of claim 307, wherein the second composition contains a polysaccharide.

491. The combination product of claim 307, wherein the first composition further comprises a carotenoid.

492. The combination product of claim 307, wherein the first composition further comprises a fragrance.

493. The combination product of claim 488, wherein the first composition contains at least one carotenoid.

494. The combination product of claim 307, wherein the microalgal polysaccharide:
a. has been made completely or partially insoluble in water through drying; and
b. has been homogenized to generate particles.

495. The combination product of claim 494, wherein the polysaccharide is contained in non-aqueous material.

496. The combination product of claim 494, wherein the polysaccharide is completely insoluble in water.

497. The combination product of claim 494, wherein the polysaccharide is partially insoluble in water.

498. The combination product of claim 494, wherein the polysaccharide is soluble in water at a percentage selected from the group consisting of less than 70%, less than 60%, less than 50%, less than 40%, less than 30%, less than 20%, less than 10%, less than 5%, and less than 2%.

499. The combination product of claim 494, wherein the particles increase in volume upon contact with water compared to their anhydrous volume.

500. The combination product of claim 494, wherein the particles increase in volume upon contact with water by an amount selected from the group consisting of at least 5%, at least 25%, at least 50%, at least 100%, at least 200%, at least 300%, at least 500%, at least 1000%, and at least 5000%.
